(19) United States
(12) Patent Application Publication
(10) Pub. No.: US 2007/0231814 A1
(43) Pub. Date: Oct. 4, 2007

(54) DNA sequences isolated from human colonic epithelial cells

(75) Inventors: Bruce M. Boman, Gladwyne, PA (US); Laingjun Wang, Plano, TX (US)

Correspondence Address: EDWARDS ANGELL PALMER & DODGE LLP, P.O. BOX 55874, BOSTON, MA, 02205, US

(73) Assignee: CA*TX, INC.

(21) Appl. No.: 11/603,483

(22) Filed: Nov. 21, 2006

Related U.S. Application Data
(63) Continuation of application No. 10/089,887, filed on Mar. 29, 2002, now abandoned.
Application No. 10/089,887 is a continuation of application No. PCT/US00/21606, filed on Aug. 8, 2000.
(60) Provisional application No. 60/147,933, filed on Aug. 9, 1999.

(51) Int. Cl. C12Q 1/68; A01K 67/027; A61K 38/00; C07H 21/04; C07K 14/00; G01N 33/53; C07K 16/18; C12N 15/00; C12N 5/06

(52) U.S. Cl. 435/6; 435/320.1; 435/325; 435/7.1; 514/2; 530/350; 530/387.1; 536/23.5; 536/24.31; 800/13

(57) ABSTRACT

The present invention discloses novel nucleic acid sequences which are implicated in the growth regulation of the epithelial cells of the colon, and which sequences are differentially expressed in cancerous colon tissues compared to normal colon tissues. These sequences are useful in diagnosing abnormal cell growth, treatment of abnormal cell growth and screening assays for, treatments of abnormal cell growth.

### RELATED APPLICATIONS

This application is a continuation of International Application No. PCT/US00/21606, which designated the United States and was filed on Aug. 8, 2000, published in English, which claims the benefit of U.S. Provisional Application No. 60/147,933. The entire teachings of the above applications are incorporated herein by reference.

### FIELD OF THE INVENTION

The present invention provides novel genes and other nucleic acid sequences that are involved in growth regulation in the human colonic epithelium, particularly those that may be involved, in carcinogenesis. The invention further provides use of such nucleic acid sequences and polypeptides and proteins encoded by them in diagnosis and treatment of diseases associated with aberrant growth regulation of human colonic epithelium.

### BACKGROUND OF THE INVENTION

It is fairly well established that many pathological conditions, such as infections, cancer and autoimmune disorders are characterized by the inappropriate over- or under-expression of certain molecules. These molecules thus can serve as markers for a particular pathological or abnormal condition. Apart from their use as diagnostic targets, i.e., materials to be identified to diagnose these abnormal conditions, the molecules may serve as reagents which can be used to generate diagnostic and/or therapeutic agents. A non-limiting example of this is the use of cancer markers to produce antibodies specific to a particular marker.

The gastrointestinal (GI) tract is the most common site of both newly diagnosed cancers and fatal cancers occurring each year in the US. The incidence of colon cancer in the USA is increasing, while that of gastric cancer is decreasing and cancer of the small intestine is rare. The incidence of gastrointestinal cancers varies geographically. In addition to environmental carcinogenic factors such as aflatoxin, certain disorders may predispose to cancer, for example, pernicious anemia to gastric cancer, untreated non-tropical sprue and immune defects to lymphoma and carcinoma, and ulcerative and granulomatous colitis, isolated polyps, and inherited familial polyposis to carcinoma of the colon. The most common tumor of the colon is adenomatous polyp. Primary lymphoma is rare in the colon and most common in the small intestine.

Adenomatous polyps are the most common benign GI tumors. They occur throughout the GI tract, most commonly in the colon and stomach, and are found more frequently in males than in females. They may bee single, or more commonly multiple, land sessile or pedunculated. They may be inherited, as in familial polyposis and Gardener's syndrome, which primarily involves the colon. Development of colon cancer is common in familial polyposis. Polyps often cause bleeding, which may occult or gross, but rarely cause pain unless complications ensue. Papillary adenoma, a less common form found only in the colon, may also cause electrolyte loss and mucoid discharge.

A malignant tumor includes a carcinoma of the colon which may be infiltrating or exophytic and occurs most commonly in the rectosigmoid. Because the content of the ascending colon is liquid, a carcinoma in this area usually does not cause obstruction, but the patient tends to present late in the course of the disease with anemia, abdominal pain, or abdominal mass or palpable mass.

The prognosis with colonic tumors depends on the degree of bowel wall invasion and on the presence of regional lymph node involvement and distant metastases. The prognosis with carcinoma of the rectum and descending colon is quite unexpectedly good. Cure rates of 80 to 90% are possible with early resection before nodal invasion develops. For this reason, great care must be taken to exclude this disease when unexplained anemia, occult gastrointestinal bleeding, or change in bowel habits develops in a previously healthy patient. Complete removal of the lesion before it spreads to the lymph nodes provides the best chance of survival for a patient with cancer of the colon. Detection in an a symptomatic patient by occult-bleeding, blood screening results in the highest five year survival.

Clinically suspected malignant lesions can usually be detected radiologically. However, polyps less than 1 cm can easily be missed, especially in the upper sigmoid and in the presence of diverticulosis. Clinically suspected and radiologically detected lesions in the esophagus, stomach or colon can be confirmed by fiber optic-endoscopy combined with histologic tissue diagnosis made by directed biopsy and brush sitology. Colonoscopy is another method utilized to detect colon diseases. Benign and malignant polyps not visualized by X-ray are often detected on colonoscopy. In addition, patients with one lesion on X-ray often have additional lesions detected on colonoscopy. Sigmoidoscope examination, however, only detects about 50% of colonic tumors.

The above described methods of detecting colon cancer have drawbacks, for example, small colonic tumors may be missed by all of these. The importance of early detection of colon cancer is also extremely important to prevent metastases. Also, specific detection methods are needed to distinguish between various types of tumors which may respond differently to treatment strategies.

### SUMMARY OF THE INVENTION

The present invention discloses novel nucleic acid sequences which are implicated in the growth regulation of the epithelial cells of the colon and which sequences are differentially expressed in the normal and cancerous tissues. These sequences are useful in diagnosing abnormal cell growth, treatment of abnormal cell growth and screening assays for treatments of abnormal cell growth.

In one embodiment, the invention provides an isolated nucleic acid comprising a nucleotide sequence of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto.

In a related embodiment, the nucleic acid is at least about 80% or more and up to and including 100% identical to a sequence corresponding to at least about 12, at least about 15, at least about 25, or at least about 40 or more consecutive-nucleotides up to the full length of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto or up to the full length open reading frame of the gene of which said sequence is a fragment.

In yet another embodiment, the nucleic acid is at least about 80% or more and up to and including 100% identical to a sequence corresponding to one of SEQ ID NO: 27; SEQ ID NO: 29; SEQ ID NO: 31; SEQ ID NO: 33; SEQ ID NO: 5; SEQ ID NO: 37; SEQ ID NO: 38 and encodes a polypeptide or protein which interacts in the yeast two-hybrid system with APC.

In yet another embodiment, the nucleic acid is at least about 80% or more and up to and including 100% identical to a sequence corresponding to one of SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 and encodes a polypeptide or protein which interacts in the yeast two-hybrid-system with E12.

In another aspect, the invention provides an isolated nucleic acid comprising a nucleotide sequence which hybridizes under stringent conditions to a sequence of one of SEQ ID NO: 27; SEQ ID NO: 29; SEQ ID NO:31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto. In a related embodiment, the nucleic acid is at least about 80% or more and up to and including 100% identical to a sequence corresponding to at least about 12, at least about 15, at least about 25, or at least about 40 or more consecutive nucleotides up to the full length of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or up to the full length of the open reading frame of the gene of which said sequence is a fragment.

In one embodiment, the invention provides a nucleic acid comprising a nucleotide sequence of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61; or a sequence complementary thereto, and a transcriptional regulatory sequence operably linked to the nucleotide sequence to render the nucleotide sequence suitable for use as an expression vector. In another embodiment, the nucleic acid may be included in an expression vector capable of replicating in a prokaryotic eukaryotic cell. In a related embodiment, the invention provides a host cell transfected with the expression vector.

In one embodiment, the invention provides a nucleic acid comprising a nucleotide sequence which hybridizes under stringent conditions to a sequence of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO 40; SEQ ID NO: 42; SEQ ID NO: 44; SEQ ID NO: 46; SEQ ID NO: 48, SEQ ID NO: 50; SEQ ID NO: 51; SEQ ID NO: 52; SEQ ID NO: 60; SEQ ID NO: 61, or a sequence complementary thereto and a transcription regulatory sequence operably linked to the nucleotide sequence to render the nucleotide sequence suitable for use as an expression vector. In another embodiment, the nucleic acid may be included in an expression vector capable of replicating in a prokaryotic or eukaryotic cell. In a related embodiment, the invention provides a host cell transfected with the expression vector.

In another embodiment, the invention provides a transgenic animal having a transgene of a nucleic acid comprising a nucleotide sequence of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto incorporated in cells of such transgenic animal. The transgene modifies the level of expression of the nucleic acid, the stability of a mRNA transcript of the nucleic acid, or the activity of the encoded product of the nucleic acid.

In another embodiment, the invention provides a transgenic animal having a transgene of a nucleic acid comprising a nucleotide sequence which hybridizes under stringent conditions to a sequence of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto incorporated in cells of such transgenic animal. The transgene modifies the level of expression of the nucleic acid, the stability of a mRNA transcript of the nucleic acid, or the activity of the encoded product of the nucleic acid.

In further embodiment, the invention provides substantially pure nucleic acid which comprises one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto or up to the full length of the open reading frame of a gene of which said sequence is a fragment.

In yet another embodiment, the invention provides substantially pure nucleic acid which hybridizes under stringent conditions to a nucleic acid probe corresponding to at least about 12, at least about 15, at least about 25, or at least about 40 or more consecutive nucleotides up to the full length of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto or up to the full length of the open reading frame of a gene of which said sequence is a fragment.

The invention also provides an antisense oligonucleotide comprising at least 12, at least 25, or at least 50 or more consecutive nucleotides of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 up to the full length of one of the SEQ ID Nos listed above, or a sequence complementary thereto or up to the full length of the open reading frame of the gene of which said sequence is a fragment. In one embodiment the antisense oligonucleotide is resistant to cleavage by a nuclease, preferably an endogenous, endonuclease or exonuclease.

The invention also provides an antisense oligonucleotide which hybridizes under stringent conditions to at least 12, at least 25, or at least 50 or more consecutive nucleotides of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50;SEQ ID NO: 51; SEQ ID NO: 52; SEQ ID NO: 60; SEQ ID NO: 61 up to the full length of one of the SEQ ID Nos listed above, or a sequence complemtentary thereto or up to the full length of the open reading frame of the gene of which said sequence is a fragment. In one embodiment the antisense oligonucleotide is resistant to cleavage by a nuclease, preferably an endogenous endonuclease or exonuclease.

In another embodiment, the inventions provides a probe/primer comprising a substantially purified oligonucleotide, said oligonucleotide containing a region of nucleotide sequence comprising at least about 12, at least about 15, at least about 25, or at least about 40 or more consecutive nucleotides of sense or antisense sequence selected from SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, up to the full length of one of the SEQ ID Nos. listed above, or a sequence complementary thereto or up to the full length of the open reading frame of a gene of which said sequence is a fragment. In preferred embodiments, the probe selectively hybridizes with a target nucleic acid. In another embodiment, the probe may include a label group attached thereto and able to be detected. The label group may be selected from a group including but not limited to radioisotopes, fluorescent compounds, enzymes, and enzyme co-factors. The invention further provides arrays of at least about 10, at least about 25, at least about 50, or at least about 100 or more different probes as described above attached to a solid support.

In another embodiment, the invention provides a probe/primer comprising a substantially purified oligonucleotide, said oligonucleotide containing a region of nucleotide sequence which hybridizes under stringent conditions to at least about 12, at least about 15, at least about 25, or at least about 40 or more consecutive nucleotides of sense or antisense sequence selected from SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, up to the full length of one of SEQ ID Nos listed above, or a sequence complementary thereto or up to the full length of the open reading of a gene of which said sequence is a fragment. In preferred embodiments, the probe selectively hybridizes with a target nucleic acid. In another embodiment, the probe may include a label group attached thereto and able to be detected. The label group may be selected from a group including but not limited to radioisotopes fluorescent compounds, enzymes, and enzyme co-factors. The invention further provides arrays of at least about 10, at least about 25, at least about 50, or at least about 100 or more different probes as described above attached to a solid support.

In yet another embodiment, the invention pertains to a method of determining the phenotype of a cell, comprising detecting the differential expression, relative to a normal cell, of at least one nucleic acid selected from SEQ ID NO: 27; SEQ ID NO: 29, SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, wherein the nucleic acid is differentially expressed by at least a factor of 2, at least a factor of 5, at least a factor of 20, or at least a factor of 50 or more.

In yet another embodiment, the invention pertains to a method of determining the phenotype of a cell, comprising detecting the differential expression, relative to a normal cell, of at least one nucleic acid which hybridizes under stringent conditions to at least one of SEQ ID NO: 27; SEQ ID NO: 29; SEQ ID NO: 31; SEQ ID NO: 33; SEQ ID NO: 35; SEQ ID NO: 37 SEQ ID NO: 38; SEQ ID NO: 40; SEQ ID NO: 42; SEQ ID NO: 44; SEQ ID NO: 46; SEQ NO: 48; SEQ ID NO: 50; SEQ ID NO: 51; SEQ ID NO: 52; SEQ ID NO: 60; SEQ ID NO wherein the nucleic acid is differentially expressed by at least a factor of 2, at least a factor of 5, at least a factor of 20, or at least a factor of 50 or more.

In another aspect, the invention provides polypeptides encoded by the subject nucleic acids. In one embodiment, the invention pertains to a polypeptide including an amino acid sequence encoded by a nucleic acid comprising a nucleotide sequence of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto, or a fragment comprising at least about 25, or at-least about 40 or more amino acids thereof. Further provided are antibodies immunoreactive with these polypeptides.

In another aspect, the invention provides polypeptides encoded by the subject nucleic acids; In one embodiment, the invention pertains to a polypeptide including an amino acid sequence encoded by a nucleic acid comprising a nucleotide sequence which hybridizes under stringent conditions to a sequence of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto, or a fragment comprising at least about 25, or at least about 40 or more amino acids thereof; Further provided are antibodies immunoreactive with these polypeptides.

In still another aspect, the invention provides diagnostic methods. In one embodiment, the invention pertains to a method for determining the phenotype of cells from a patient by providing one or more nucleic-acid probe(s) comprising a nucleotide sequence having at least 12, at least about 15, at least about 25, or at least about 40 or more consecutive nucleotides represented in a sequence of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 up to the full length of one of SEQ ID Nos listed above, or a sequence complementary thereto or up to the full length of the open reading frame of a gene of which said nucleic acid sequence is a fragment. The method comprising obtaining a first sample of cells from a patient, providing a second sample of cells substantially all of which are non-cancerous, contacting the nucleic acid probe under stringent conditions with mRNA of each of said first and second cell samples, and comparing (a) the amount of hybridization of the probe with mRNA of the first cell sample, with (b) the amount of hybridization of the probe with mRNA of the second cell sample, wherein a difference of at least a factor of 2, at least a factor of 5, at least a factor of 20, or at least a factor of 50 or more in the amount of hybridization with the mRNA of the first cell sample as compared to the amount of hybridization with the mRNA of the second cell sample is indicative of the phenotype of cells in the first cell sample. Determining the phenotype includes determining the genotype, as the term is used herein.

In another embodiment, the invention provides a test kit for identifying transformed cells, comprising one or more probe(s)/primer(s) as described above, for measuring a level of a nucleic acid which hybridizes under stringent conditions to a nucleic acid of one or more of SEQ ID NO:27; SEQ ID NO 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 in a sample of cells isolated from a patient. In certain embodiments, the kit may further include instructions for using the kit, solutions for suspending or fixing the cells,:,detectable tags or labels, solutions for rendering a nucleic acid susceptible to hybridization, solutions for lysing cells, or solutions for the purification of nucleic acids.

In another embodiment, the invention provides a method of determining the phenotype of a cell, comprising detecting the differential expression, relative to a normal cell, of at least one protein encoded by a nucleic acid of one of SEQ ID NO: 27; SEQ ID NO: :29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, wherein the protein is differentially expressed by at least a factor of 2, at least a factor of 5, at least a factor of 20, or at least a factor of 50 or more. In one embodiment, the level of the protein is detected in an immunoassay.

In another embodiment, the invention provides a method of determining the phenotype of a cell, comprising detecting the differential expression, relative to a normal cell, of at least one protein encoded by a nucleic acid which hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38, and which protein interacts in the yeast two-hybrid system with APC wherein the protein is differentially expressed by at least a factor of 2, at least a factor of 5, at least a factor of 20, or at least a factor of 50 or more. In one embodiment, the level of the protein is detected in an immunoassay.

In another embodiment, the invention provides a method of determining the phenotype of a cell, comprising detecting the differential expression, relative to a normal cell, of at least one protein encoded by a nucleic acid which hybridizes under stringent conditions.-to one of SEQ ID NO: 40; SEQ ID NO: 42; SEQ ID NO: 44; SEQ ID NO: 46, and which protein interacts in the yeast two hybrid system with E12 wherein the protein is differentially expressed by at least a factor of 2, at least a factor of 5, at least a factor of 20, or at least a factor of 50 or more. In one embodiment, the level of the protein is detected in an immunoassay.

The invention also pertains to a method for determining the presence or absence of a nucleic acid which hybridizes under stringent conditions to one of SEQ ID No: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46, SEQ ID NO: 48; SEQ ID NO: 50; SEQ ID NO: 51; SEQ ID NO: 52; SEQ ID NO: 60; SEQ ID NO: 61 in a cell, comprising contacting the cell with one ore more probe(s)/primer(s) as described in the three previous embodiments above.

The invention further provides a method for determining the presence or absence of a subject polypeptide encoded-by a nucleic acid which hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 in a cell, comprising contacting the cell with an antibody as described above.

In yet another embodiment, the invention provides a method for determining the presence of an aberrant mutation (e.g., deletion, insertion, or substitution of nucleic acids) or aberrant methylation sequence or a gene which comprises the nucleic acid sequence of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto. The method comprising collecting a sample of cells from a patient, isolating nucleic acid from the cells of the sample, contacting the nucleic acid sample with one or more primers which specifically hybridize to a nucleic acid sequence of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO 50, SEQ ID NO: 51; SEQ ID NO: 52; SEQ ID NO: 60; SEQ ID NO: 61 under conditions which allow hybridization of the probe(s)/primer(s) with the nucleic acid and consequently amplification, of the nucleic acid, and comparing the presence, absence, or size of an amplification product to the amplification product of a normal cell.

In one embodiment, the invention provides a test kit for identifying transformed cells, comprising an antibody specific for a protein encoded by a nucleic acid of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ NO: 52; SEQ ID NO: 60; SEQ ID NO: 61. In certain embodiments, the kit further includes instructions for using the kit. In certain embodiments, the kit may further include instructions for using the kit, solutions-for suspending or fixing the cells, detectable tags or labels, solutions for rendering a polypeptide susceptible to the binding of an antibody, solutions for lysing cells, or solutions for the purification of polypeptides.

In yet another aspect, the invention provides pharmaceutical compositions including the subject nucleic acids. In one embodiment, an agent which alters the level of expression in a cell of a nucleic acid of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto is identified by providing a cell, treating the cell with a test agent, determining the level of expression in the cell of a nucleic acid which hybridizes under stringent conditions to one of SEQ ID Nos listed above or a sequence complementary thereto, and comparing the level of expression of the nucleic acid in the treated cell with the level of expression of the nucleic acid in an untreated cell, wherein a change in the level of expression of the nucleic acid in the treated cell relative to the level of expression of the nucleic acid in the untreated cell is indicative of an agent which alters the level of expression of the nucleic acid in a cell. The invention further provides a pharmaceutical composition comprising an agent identified by this method.

In another embodiment, the invention provides a pharmaceutical composition which includes a polypeptide encoded by a nucleic acid having a nucleotide sequence of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto.

In one embodiment, the invention pertains to a pharmaceutical composition comprising a nucleic acid including a sequence which hybridizes under stringent contains to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44 SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto.

In one embodiment; an agent which alters the level of expression in a cell of a nucleic acid which hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto is identified by providing a cell, treating the cell with a test agent, determining the level of expression in the cell of a nucleic acid which hybridizes under stringent conditions to one of SEQ ID Nos listed above or a sequence complementary thereto, and comparing the level of expression of the nucleic acid in the treated cell with the level of expression of the nucleic acid in an untreated cell, wherein a change in the level of expression of the nucleic acid in the treated cell relative to the level of expression of the nucleic acid in the untreated cell is indicative of an agent which alters the level of expression of the nucleic acid in a cell. The invention further provides a pharmaceutical composition comprising an agent identified by this method.

In another embodiment, the invention provides a pharmaceutical composition which includes a polypeptide encoded by a nucleic acid having a nucleotide sequence that hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52, SEQ ID NO: 60; SEQ ID NO. 61 or a sequence complementary thereto in one embodiment, the invention pertains to a pharmaceutical composition comprising a nucleic acid including a sequence which hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto.

### DETAILED DESCRIPTION OF THE INVENTION

The invention relates to-nucleic acids having the disclosed nucleic acid sequences (SEQ ID NO: 27; SEQ ID NO: 29; SEQ ID NO: 31; SEQ ID NO: 33; SEQ ID NO: 35, SEQ ID NO: 37; SEQ ID NO: 38; SEQ ID NO: 40 SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61), as well as full length cDNA, mRNA, and genes corresponding to these sequences, and to polypeptides and proteins encoded by these nucleic acids and genes, and portions thereof.

Also included are polypeptides and proteins encoded by the nucleic acids of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61. The various nucleic acids that can encode these polypeptides and proteins differ because of the degeneracy of the genetic code, in that most amino acids are encoded by more than one triplet codon. The identity of such codons is well known in this art, and this information can be used for the construction of the nucleic acids within the scope of the invention.

Nucleic acids encoding polypeptides and proteins that are variants of the polypeptides and proteins encoded by the nucleic acids and related cDNA and genes are also within the scope of the invention. The variants differ from wild-type protein in having one or more amino acid substitutions that either enhance, add, or diminish a biological activity of the wild-type protein. Once the amino acid change is selected, a nucleic acid encoding that variant is constructed and its function is determined according to the invention.

The following detailed description disclose how to obtain or make the nucleic acid sequences of the invention and full-length cDNA and human genes corresponding to the nucleic acids, how to express these nucleic acids and genes, how to identify interactions of a polypeptide or a protein encoded by a gene corresponding to a nucleic acid, how to use nucleic, acids as probes in tissue profiling, how to use the corresponding polypeptides and proteins to raise antibodies, and how to use the nucleic acids, polypeptides, and proteins for diagnostic and therapeutic purposes.

The sequences disclosed herein have been, found to be differentially expressed in samples obtained from colon cancer cell lines and/or colon cancer tissue. Moreover, the proteins encoded by the nucleic acid sequences designated CATX1, CATX2, CATX3, CATX4, CATX5, CATX6, and CATX7 interact at least with the APC gene; the proteins encoded by nucleic acid sequences of CATX11, CATX12, CATX13, CATX14, CATX15 interact with at least the E12 gene; and the nucleic acid sequences of CATX8, CATX9, CATX10 are homologous to the SCL gene. These specific interactions and homologies with different cancer associated genes suggest that the disclosed sequences also have utility with other types of cancer.

Accordingly, certain aspects of the present invention relate to nucleic acids differentially expressed in tumor tissue, especially colon cancer cell lines, polypeptides encoded by such nucleic acids, and antibodies immunoreactive with these polypeptides, and preparations of such compositions. Moreover, the present invention provides diagnostic and therapeutic assays and reagents for detecting and treating disorders involving, for example, aberrant expression of the subject nucleic acids.

The following abbreviations are used herein:

APC—adenomatous polyposis coli gene
DNA—deoxyribonucleic acid
RNA—ribonucleic acid
cDNA—complementary DNA
complete cDNA—a cDNA that contains the complementary sequences corresponding to the full length messenger RNA molecule
PCR—polymerase chain reaction
bHLH—basic helix-loop-helix.
E12 gene encoding a-transcription factor containing a bHLH motif
SCL—stem cell leukemia gene
   General

This invention relates in part to novel methods for identifying and/or classifying cancerous cells present in a human tumors, particularly in solid tumors, e.g., carcinomas and sarcomas, such as for example breast or colon cancers. The method is based on the discovery that certain nucleic acid sequences are differentially expressed in cancer cell, lines and/or cancer tissue compared with elated normal cells, such as normal colon cells, and thereby identifies or classifies tumor cells by the upregulation and/or downregulation of expression of particular sequences, an event which is implicated in tumorigenesis.

Upregulation or increased expression of certain genes such as oncogenes, acts to promote malignant growth. Downregulation or decreased expression of genes such as tumor suppress or genes also promotes malignant growth. Thus, alteration in the expression of either type of gene is a potential diagnostic indicator for determining whether a subject is at risk of developing or has cancer, in particular, colon cancer.

Accordingly, in one aspect, the invention also provides biomarkers, such as nucleic acid markers, for human tumor cells, e.g., for colon cancer cells. The invention also provides proteins encoded by these nucleic acid markers. The invention also features methods for identifying drugs useful for treatment of such cancer cells, and for treatment of a cancerous condition, such as colon cancer. Unlike prior methods, the invention provides a means for identifying cancer cells at an early stage of development, so that premalignant cells can be identified prior to their spreading throughout the human body. This allows early detection of potentially cancerous conditions, and treatment of those cancerous conditions prior to spread of the cancerous cells throughout the body, or prior to development of an irreversible cancerous condition.

Definitions

For convenience, the meaning of certain terms and phrases used in the specification, examples, and appended claims, are provided below.

The term “an aberrant expression”, as applied to a nucleic acid of the present invention, refers to level of expression of that nucleic acid which differs from the level of expression of that nucleic acid in healthy tissue, or which differs from the activity of the polypeptide present in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in the activity; for example, an aberrant polypeptide can interact with a different target peptide. A cell can have an aberrant expression level of a gene due to overexpression or underexpression of that gene.

The term “agonist”, as used herein, is meant to refer to an agent that mimics or upregulates (e.g., potentiates or supplements) the bioactivity of a protein. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide with another molecule, e.g., a target peptide or nucleic acid.

The term “allele”, which is used interchangeably herein with “allelic variant”, refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide, or several nucleotides, and can include substitutions, deletions, and/or insertions of nucleotides. An allele of a gene can also be a form of a gene containing mutations.

The term “allelic variant of a polymorphic region of a gene” refers to a region of a gene having one of several possible nucleotide sequences found in that region of the gene in different individuals.

“Antagonist” as used herein is meant to refer to an agent that downregulates (e.g., suppresses or inhibits) at least one bioactivity of a protein. An antagonist can be a compound which inhibits or decreases the interaction between a protein and another molecule, e.g., a target peptide or enzyme substrate. An antagonist can also be a compound that downregulates expression of a gene or which reduces the amount of expressed protein present.

The term “antibody” as used herein is intended to include whole antibodies of any isotype (IgG, IgA, IgM, IgEP, etc), and fragments thereof which are also specifically reactive with a vertebrate, e,g., mammalian, protein. Antibodies can be fragmented using conventional techniques and the fragments screened for utility in the same manner as whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal monoclonal, or other purified preparations of antibodies and recombinant, including but not limited to humanized antibodies.

The phenomenon of “apoptosis” is well known, and can be described as a programmed death of cells. As is known, apoptosis is contrasted with “necrosis”, a phenomenon when cells die as a result of being killed by a toxic material, or other external effect. Apoptosis involves chromatic condensation, membrane blebbing, and fragmentation of DNA, all of which are generally visible upon microscopic examination.

A disease, disorder, or condition “associated with” or “characterized by” an aberrant expression of a nucleic acid refers to a disease, disorder, or condition in a subject individual, which disease, disorder or condition is caused by, contributed to by, or causative of an aberrant level of expression of a nucleic acid.

As used herein the term “bioactive fragment of a polypeptide” refers to a fragment of a full-length polypeptide, wherein the fragment specifically agonizes (mimics) or antagonizes (inhibits) the activity of a wild-type polypeptide. The bioactive fragment preferably is a fragment capable of interacting with at least one other molecule, e.g., protein, small molecule, or DNA, which a full length protein can bind.

“Biological activity” or “bioactivity” or “activity” or “biological function”, which are used interchangeably, herein mean an effector or antigenic function that is directly or indirectly performed by a:polypeptide (whether in its native or denatured conformation), or by any subsequence thereof. Biological activities include binding to polypeptides, binding to other proteins or molecules, activity as a DNA binding protein, as a transcription regulator, ability to bind damaged DNA, etc. A bioactivity can be modulated by directly affecting the subject polypeptide. Alternatively, a bioactivity can be altered by modulating the level, of the polypeptide, such as by modulating expression of the corresponding gene.

The term “biomarker” refers a biolgical molecule, e.g., a nucleic acid, peptide, hormone, etc; whose presence or concentration can be detected and correlated with a known condition, such as a disease state.

“Cells,” “host cells”, or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric polypeptide” or “fusion polypeptide” is a fusion of a first amino acid sequence encoding one of the subject polypeptides with a second amino acid sequence defining a domain (e.g., polypeptide portion) foreign to and not substantially homologous with any domain of the subject polypeptide. A chimeric polypeptide may present a foreign domain which is found (albeit in a different polypeptide) in an organism which also expresses the first polypeptide, or it may be an “interspecies,” “intergenic,” etc., fusion of polypeptide structures expressed by different kinds of organisms.

A “delivery complex” shall mean-a targeting means (e.g., a molecule that results in higher affinity binding of a nucleic acid, protein, polypeptide or peptide to a target cell surface and/or increased cellular or nuclear uptake by a target cell). Examples of targeting means include: sterols (e.g., cholesterol), lipids (e.g., a cationic lipid, virosome or liposome), viruses (e.g., adenovirus, adeno-associated virus, and retrovirus), or target cell-specific binding agents (eg., ligands recognized, by target cell specific receptors). Preferred complexes are sufficiently stable in vivo to prevent significant uncoupling prior to internalization by the target cell. However, the complex is cleavable under appropriate conditions outside or within the cell, so that the nucleic acid, protein, polypeptide or peptide is released in a functional form.

As is well known, genes or a particular polypeptide may exist in single or multiple copies within the genome of an individual. Such duplicate genes may be identical or may have certain modifications, including nucleotide substitutions, additions or deletions, which all still code for polypeptides having substantially the same activity. The term “DNA-sequence encoding a polypeptide” may thus refer to one or more nucleic acid sequences within a particular individual. Moreover, certain differences in nucleotide sequences may exist between individual organisms, which are called alleles. Such allelic differences may or may not result in differences in amino acid sequence of the encoded polypeptide yet still encode a polypeptide with the same biological activity.

The term, “equivalent” is understood to include nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of the nucleic acids shown in SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 due to the degeneracy of the genetic code.

As used herein, the terms “gene”, “recombinant gene”, and “gene construct” refer to a nucleic acid of the present invention associated with an open reading frame, including exon and optionally also intron sequences.

A “recombinant gene” refers to nucleic acid encoding a polypeptide and comprising exon sequences, though it may optionally include intron sequences which are derived from, for example, a related or unrelated chromosomal gene. The term “intron” refers to a DNA sequence present in a given gene which is not translated into protein and is generally found between exons.

The term “growth” or “growth state” of a cell refers to the proliferative state of a cell as well as to its differentiative state. Accordingly, the term refers to the phase of the cell cycle in which the cell is, e.g., G₀, G₁, G₂, prophase, metaphase, or telophase, as well as to its state of differentiation, e.g., undifferentiated, partially differentiated, or fully differentiated. Without wanting to be limited, differentiation of a cell is usually accompanied by a decrease in the proliferative rate of a cell.

“Homology” or “identity” or “similarity” refers to, sequence similarity between two peptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e., structurally related, at positions shared by the amino acid sequences. An “unrelated” or “non-homologous” sequence shares less than 40%- identity, though preferably less than 25% identity, with one of the sequences of the present invention.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including but not limited to FASTA, BLAST, or ENTREZ. FASTA and BLAST are available, e.g., as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In on embodiment, the percent identity of two sequences can be determined by the GCG program with a gap weight of 1, e.g., each amino acid gap is weighted as if it were a single amino acid or nucleotide mismatch between the two sequences.

Other techniques for alignment ate described in Methods in Enzymology, vol. 266: Computer Methods for Macromolecular Sequence Analysis (1996), ed., Doolittle, Academic Press, Inc., a division of Harcourt Brace & Co., San Diego, Calif., USA. Preferably, an alignment program that permits gaps in the sequence is utilized to align the sequences the Smith-Waterman is one type of algorithm that permits gaps in sequence alignments. See Meth. Mol. 70-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology. ed. Doolittle, supra. Databases include but are not limited to Genbank, EMBL, and DNA Database of Japan (DDBJ).

Preferred nucleic acids comprise one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or sequences complementary thereof. More preferred nucleic acids comprise sequences that hybridize under stringent conditions to a nucleic acid sequence of one of SEQ ID NO: 27; SEQ ID NO: 29; SEQ ID NO: 31; SEQ ID NO: 3.3; SEQ ID NO: 35; SEQ ID NO: 37; SEQ ID NO: 38, SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61. Preferred nucleic acids have a sequence at least 70%, and more preferably 80% identical and more preferably 90% and even more preferably at least 95% identical ito an nucleicacid se ce of sequence shown in one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61. Further, nucleic acids at least 90%, more preferably 95%, and most prefeyrably at least about 98-99% identical with a nucleic sequence represented in one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 146; SEQ ID NO: 48; SEQ ID NO: 50; SEQ ID NO: 51; SEQ ID NO: 52; SEQ ID NO: 60; SEQ ID NO: 61 are also within the scope of the invention. In preferred embodiments, the nucleic acid is mammalian.

The term “interact” as used herein is meant to include detectable interactions (e.g., biochemical interactions) between molecules, such as interaction between protein-protein, protein-nucleic acid, nucleic acid-nucleic acid, and protein-small molecule or nucleic acid-small molecule in nature.

The term “isolated” as used herein with respect to nucleic acids, such as DNA or RNA, refers to molecules separated from other DNAs, or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The terms “modulated” and “differentially regulated” as used herein refer to both upregulation (i.e., activation or stimulation, e.g., by agonizing or potentiating) and downregulation (i.e., inhibition or suppression, e.g., by antagonizing, decreasing or inhibiting).

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

The designation “N”, where it appears in the accompanying Sequence Listing, indicates that the identity of the corresponding nucleotide is unknown. “N ” should therefore not necessarily be interpreted as permitting substitution with any nucleotide, e.g., A, T, C, or G, but rather as holding the place of a nucleotide whose identity has not been conclusively determined.

The “non-human animals” of the invention include mammalians such as, rodents, non-human primates, sheep, dog, cow, as well as non-mammalian animals such as chickens, amphibians, reptiles, etc. A preferred non-human animal is selected from the rodent family including rat and mouse, most preferably mouse, although transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant genes is present and/or expressed or disrupted in some tissues but not others.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides which may also be modified. ESTs, chromosomes, cDNAs, mRNAs, and rRNAs are representative examples of molecules that may be referred to as nucleic acids.

The term “nucleotide sequence complementary to the nucleotide sequence of SEQ ID NO. X” refers to the nucleotide sequence of the complementary strand of a nucleic acid strand having SEQ ID NO. X. The term. “complementary strand” is used herein interchangeably with the term “complement.” The complement of a nucleic acid strand can be the complement of a coding strand or the complement of a non-coding strand.

The term “polymorphism” refers to the coexistence of more than one form of a gene or portion (e.g., allelic variant) thereof. A portion of a gene of which there are at least two different forms, i.e., two different nucleotide sequences; is referred to as a “polymorphic region of a gene.” A polymorphic region can be a single nucleotide, the identity of which differs in different alleles. A polymorphic region can also be several nucleotides long.

A “polymorphic gene” refers to a gene having at least one polymorphic region.

As used herein, the term “promoter” means a DNA sequence that regulates expression of a selected DNA sequence operably linked to the promoter, and which effects expression of the selected DNA sequence in cells. The term encompasses “tissue specific” promoters, i.e., promoters which effect expression of the selected DNA sequence only in specific cells (e.g., cells of a specific tissue). The term also covers so-called “leaky” promoters, which regulate expression of a selected DNA primarily in one tissue, but cause expression in other tissues as well. The term also encompasses non-tissue specific promoters and promoters that constitutively expressed or that are inducible (i.e., expression levels can be controlled).

The terms “protein”, “polypeptide”, and “peptide” are used interchangeably herein when referring to a gene product.

The term “recombinant protein” refers to a polypeptide of the present invention which is produced by recombinant DNA techniques, wherein generally, DNA encoding a polypeptide is inserted into a suitable expression vector which is in turn used to transform a host cell to produce the heterologous protein. Moreover, the phrase “derived from”, with respect to a recombinant gene, is meant to include within the meaning of “recombinant protein” those proteins having an amino acid sequence of a native polypeptide, or an amino acid sequence similar thereto which is generated by mutations including substitutions and deletions (including truncation) of a naturally occurring form of the polypeptide.

“Small molecule” as used herein, is meant to refer to a composition, which has a molecular weight of less than about 5 kD and most preferably less than about 4 kD. Small molecules can be nucleic acids, peptides, polypeptides, peptidometics, carbohydrates, lipids or other organic (carbon-containing) or inorganic molecules. Many pharmaceutical companies have extensive libraries of chemical and/or biological mixtures, often fungal bacterial, or algal extracts, which can be screened with any of the assays of the invention to identify compounds that modulate a bioactivity.

“Solid phase” refers to a non-aqueous matrix to which the nucleic acid or antibody of the present invention can adhere. Examples of solid phases encompassed herein include those formed partially or entirely of glass, e.g., controlled pore glass; polysaccharides, e.g., agarose; organic polymers such as polycarbonate; polyacrylamides; polystyrene; polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

As used herein, the term “specifically hybridizes” or “specifically detects” refers to the ability of a nucleic acid molecule of the invention to hybridize to at least a portion of, for example approximately 6, 12, 15, 20, 30, 50, 100, 150, 200, 300, 350, 400, 500, 750, or 1000 contiguous nucleotides of a nucleic acid designated in any one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, or naturally occurring mutants thereof, such that it has less than 15%, preferably less than 10%, and more preferably less than 5% background hybridization to a cellular nucleic acid (e.g., mRNA or genomic DNA) encoding a different protein. In preferred embodiments, the oligonucleotide probe detects only a specific nucleic acid, e.g., it does not substantially hybridize to similar or related nucleic acids, or complements thereof.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally occurring forms of the polypeptide.

As used herein, the term “transfection” means the introduction of a nucleic acid,.e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of the target gene is disrupted.

As used herein, the term “transgene” means a nucleic acid sequence (or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical crossbreeding, or in vitro fertilization, but rather is directed to the introduction of a recombinant DNA molecule. This molecule may be integrated within a chromosome, or it may be extra-chromosomally replicating DNA. In the typical transgenic animals described herein, the transgene causes cells to express a recombinant form of one of the subject polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

The term “treating” as used herein is intended to encompass curing as well as ameliorating at least one symptom of the condition or disease.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operably linked are referred to herein as “expression vectors”. In general, expression vectors of utility in recombinant DNA techniques are often in the form of “plasmids” which refer generally to circular double stranded DNA molecules which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

Nucleic Acids of the Present Invention

As described below, one aspect of the invention pertains to, isolated-nucleic acids, variants, and/or equivalents of such nucleic acids.

Nucleic acids of the present invention including SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44, SEQ ID NO: 46; SEQ ID NO: 48, SEQ ID NO: 50; SEQ ID NO: 51; SEQ ID NO: 52, SEQ ID NO: 60; SEQ ID NO: 61, or a sequence complementary thereto, have been identified as differentially expressed in tumor cells; e.g., colon cancer-derived cell-lines (relative to the expression levels in normal tissue, e.g., normal colon tissue and/or normal non-colon tissue. In certain embodiments, the subject nucleic, acids are differentially expressed by at least a factor of 2, preferably at least a factor of 5, even more preferably at least,a factor of 20, still more preferably at least a factor of 50 or more. Preferred nucleic acids include sequences identified as differentially expressed both in colon cancer cell tissue and colon cancer cell lines. In preferred embodiments, nucleic acids of the present invention are upregulated in tumor cells, especially colon cancer tissue and/or colon cancer-derived cell lines. In another embodiment, nucleic acids of the present invention are downregulated in tumor cells, especially colon cancer tissue and/or colon cancer-derived cell lines.

Particularly preferred polypeptides are those that are encoded by nucleic acid sequences at least about 70%, 75%, 80%, 90%, 95%, 97%, or 98% similar to a nucleic acid sequence of SEQ ID NO: 27; SEQ iDD NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ II) NO: 60; SEQ ID NO: 61. Preferably, the nucleic acid includes all or a portion (e.g., at least about 12, at least about 15, at least about 25, or at least about 40 or more nucleotides) of the nucleotide sequence corresponding to the nucleic acid of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto.

Still other preferred nucleic acids of the present invention encode a polypeptide comprising at least a port ion of a-polypeptide encoded by one of SEQ ID NO: 27; SEQ ID NO: 29; SEQ ID NO: 31; SEQ ID NO:. 33; SEQ ID NO: 35; SEQ ID NO: 37; SEQ ID NO 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO 52; SEQ ID NO: 60; SEQ ID NO: 61. For example, preferred nucleic acid molecules for use as probes/primers or antisense molecules (i.e., noncoding nucleic acid molecules) can comprise at least about 12, 20, 30, 50, 60, 70, 80, 90, or 100 base pairs in length up to the length of the complete gene. Coding nucleic acid molecules can, comprise, for example, from about 50, 60, 70; 80, 90, or 100 or more base pairs up to the length of the complete gene.

Another aspect of the invention provides a nucleic acid which hybridizes under low, medium, or high stringency conditions to a nucleic acid sequence represented by one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto. Appropriate stringency conditions which promote DNA hybridization, for example, 6.0 ×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-12.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In a preferred embodiment, a nucleic acid of the present invention will bind to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, under moderately stringent conditions, for example at- about 2.0×SSC and about 40° C. In a particularly preferred embodiment, a nucleic acid of the present invention will bind to one of SEQ ID NO: 27; SEQ ID NO: 29; SEQ ID NO: 31; SEQ ID NO: 33; SEQ ID NO: 35; SEQ ID NO: 3; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, under high stringency conditions.

In one embodiment, the invention provides nucleic acids which hybridize under low stringency conditions of 6×SSC at room temperature followed by a wash at 2×SSC at room temperature.

In another embodiment, the invention provides nucleic acids which hybridize under high stringency conditions of 2×SSC at 65° C. followed by a wash at 0.2×S SC at 65° C.

Nucleic acids having a sequence that differs from the nucleotide sequences shown in one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO. 51; SEQ ID NO: 52; SEQ ID NO: 60; SEQ ID NO: 61, or a sequence complementary thereto, due to degeneracy in the genetic code, are also within the scope of the invention. Such nucleic acids encode functionally-equivalent peptides. (i.e., a peptide having equivalent or similar biological activity) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of a polypeptide. However, it is expected that DNA sequence polymorphisms that do lead to changes in the amino acid sequences of the subject polypeptides will exist among mammals. One skilled in the art will appreciate that these variations in one or more nucleotides (e.g., up to about 3-5% of the nucleotides) of the nucleic acids encoding polypeptides having an activity of a polypeptide may exist among individuals of a given species due to natural allelic variation.

Also within the scope of the invention are nucleic acids encoding splicing variants of proteins encoded by a nucleic acid of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, or natural homologs of such proteins. Such homologs can be cloned by hybridization or PCR, as further described herein.

The polynucleotide sequence may also encode for a leader sequence, e.g., the natural leader sequence or a heterologous leader sequence, for a subject polypeptide. For example, the desired DNA sequence may be fused in the same reading frame to a DNA sequence which aids in expression and secretion of the polypeptide from the host cell, for example, a leader sequence which functions as a secretory sequence for controlling transport of the polypeptide from the cell. The protein having a leader sequence is a preprotein and may have the leader sequence cleaved by the host cell to form the mature form of the protein.

The polynucleotide of the present invention may also be fused in frame to a marker sequence, also referred to herein as “Tag sequence” encoding a “Tag peptide”, which allows for marking and/or purification of the present invention. In a preferred embodiment, the marker sequence is a hexahistidine tag, e.g., supplied by a PQE-9 vector. Numerous other Tag peptides are available commercially. Other frequently used Tags include myc-epitopes (e.g., see Ellison et al. (1991) J Biol hem 266:21150-2 1157) which includes a 10-residue sequence from c-myc, the pFLAG system (International Biotechnologies, Inc.), the pEZZ-protein A system (Pharmacia, N.J.), and a 16 amino acid portion of the Haemophilus influenza hemagglutinin protein. Furthermore, any polypeptide can be used as a Tag so long as a reagent, e.g., an antibody interacting specifically with the Tag polypeptide is available or can be prepared or identified.

As indicated by- the examples set out below, nucleic acids can be obtained from mRNA present in any of a number of eukaryotic cells and are preferably obtained from metazoan cells, more preferably from vertebrate cells, and even more preferably from mammalian cells. It should also be possible to obtain nucleic acids of the present invention from genomic DNA from both adults and embryos. For example, a gene can be cloned from either a cDNA or a genomic library in accordance with protocols generally known to persons skilled in the art. cDNA can be obtained by isolating total mRNA from a cell, e.g., a vertebrate cell, a mammalian cell, or a human cell, including embryonic cells. Double stranded cDNAs can then be prepared from the total mRNA, and subsequently inserted into a suitable plasmid or bacteriophage vector using any one of a number of known techniques. The gene can also be cloned using established PCR amplification techniques in accordance with the nucleotide sequence information provided by the invention.

The invention includes within its scope a polynucleotide having the nucleotide sequence of nucleic acid obtained from this biological material, wherein the nucleic acid hybridizes under stringent conditions (at least about 4×SSC at 65° C., or at least about 4×SSC at, 42° C., as described, for example, in U.S. Pat. No. 5,707,829, incorporated herein by reference) with at least 15 contiguous nucleotides of at least one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 4.2; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61. By this is intended that when at least 15 contiguous nucleotides of one of SEQ ID NO: 27; SEQ ID NO: 29; SEQ ID NO: 31; SEQ ID NO: 33; SEQ:ID NO: 35; SEQ ID NO: 37; SEQ ID NO: 38; SEQ ID NO: 40; SEQ ID NO: 42; SEQ ID NO: 44; SEQ ID NO. 46; SEQ ID NO: 48; SEQ ID NO: 50; SEQ ID NO: 51; SEQ ID NO: 52; SEQ ID NO: 60; SEQ ID NO: 61 is used as a probe, the probe will preferentially hybridize with a gene or mRNA (of the biological material) comprising the complementary sequence, allowing the identification and retrieval of the nucleic acids of the biological material that uniquely hybridize to the selected probe. Probes from more than one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 will hybridize with the same gene or mRNA if the cDNA from which they were derived corresponds to one mRNA. Probes of more than 15 nucleotides can be used, but 15 nucleotides represents enough sequence for unique identification.

The invention further entails a peptide or protein which comprises an amino acid sequence encoded by a nucleic acid sequence of at least 80% homology to one of SEQ ID NO: 27; SEQ ID NO: 29; SEQ ID NO: 31; SEQ ID NO: 33; SEQ ID NO: 35; or SEQ ID NO: 37; SEQ ID NO: 38 and which peptide or protein further interact with APC in a yeast two-hybrid system.

The invention further entails a peptide or protein which comprises an amino acid sequence encoded by a nucleic acid sequence of at least 80% homology to one of SEQ ID NO: 40; SEQ ID NO: 42; SEQ ID NO:44; or SEQ ID NO: 46 and which peptide or protein further interact with E12 in a yeast two-hybrid system.

Because some of the present nucleic acids represent partial mRNA transcripts, two or more nucleic acids of the invention may represent different regions of the same mRNA transcript and the same gene. Thus, if two or more of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 are identified as belonging to the same clone, then either sequence can be used to obtain the full-length mRNA or gene. Nucleic acid-related polynucleotides can also be isolated from cDNA libraries. These libraries are preferably prepared from mRNA of human colon cells, more preferably, human colon cancer specific tissue. In another embodiment the nucleic acids are isolated from libraries prepared from normal colon specific tissue. In yet another embodiment, this invention discloses nucleic acid sequences that can be isolated from both libraries prepared from a human colon carcinoma cell line, HCT116 (ATCC.No. CCL-247), as well as from libraries prepared from either normal colon specific tissue or from colon cancer specific tissue. These sequences are listed in as SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 in the. Sequence Listing. Alignment of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, as described above, can indicate that a cell line or tissue source of a related protein or polynucleotide can also be used as a source of the nucleic acid-related cDNA.

Techniques for producing and probing nucleic acid sequence libraries are described, for example, in Sambrook et al., “Molecular Cloning: A Laboratory Manual” (New York, Cold Spring Harbor Laboratory, 1989). The cDNA can be prepared by using primers based on a sequence from SEQ ID NO: 27; SEQ ID NO: 29; SEQ ID NO: 31; SEQ ID NO: 33; SEQ ID NO: 35; SEQ ID NO: 37; SEQ ID NO; 38; SEQ ID NO: 40; SEQ ID NO: 42; SEQ ID NO: 44; SEQ ID NO: 46; SEQ ID NO:. 48; SEQ ID NO: 50, SEQ ID NO: 51; SEQ ID NO: 52; SEQ ID NO: 60; SEQ ID NO: 61. In one embodiment, the cDNA library can be made from only poly-adenylated mRNA. Thus, poly-T primers can be used to prepare cDNA from the mRNA. Alignment of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 can result in identification of a related polypeptide or polynucleotide. Some of the polynucleotides disclosed herein contains repetitive regions that were subject to masking during the search procedures. The information about the repetitive regions is-discussed below.

Constructs of polynucleotides having, sequences of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46, SEQ ID NO: 48; SEQ ID NO: 50; SEQ ID NO: 51; SEQ ID NO: 52; SEQ ID NO: 60; SEQ ID NO: 61 can be generated synthetically. Alternatively, single step assembly of a:gene and entire plasmid from large numbers of oligodeoxyribonucleotides is described by Stemmer et al, Gene (Amsterdam) (1995) 164(i):49-53. In this method, assembly PCR (the synthesis of long DNA sequences from large numbers of oligodeoxyribonucleotides (oligos)) is described. The method is derived from DNA shuffling (Stemmer, Nature (1994) 370:389-391), and does not rely on DNA ligase, but instead relies on DNA polymerase to build increasingly longer DNA fragments during the assembly process. For example, a 1.1-kb fragment containing the TEM-1 beta-lactamase-encoding gene (bla) can be assembled in a single reaction from a total of 56 oligos, each 40 nucleotides (nt) in length. The synthetic gene can be PCR amplified and cloned in a vector containing, e.g., the tetracycline-resistance gene (Tc-R) as the sole selectable marker. Without relying on ampicillin (Ap) selection, 76% of the Tc-R colonies were Ap-R, making this approach a general method for the rapid and cost-effective synthesis of any gene.

Identification of Functional and Structural Motifs of Novel Sequences Using Art-Recognized Methods

The nucleic acids of the present invention were identified by their interaction with other nucleic acids or proteins using cDNA libraries in:yeast two-hybrid system or low-stingency PCR. The yeast two-hybrid assay used in the present invention was based on a process developed by Fields and coworkers (Nature, 340:245-247,1989). The yeast two-hybrid assay is a yeast-based genetic assay designed to detect protein protein interactions in vitro. A positive result obtained with the two-hybrid assay allows detection of the presence of genes, for example from a cDNA library, which genes encode candidate proteins that interact with, a target protein (“bait”). The method is based on the fact that many eukaryotic transcriptional activator proteins consist of two physically separable domains: one acts as the, DNA binding domain (“BD”) and the other as the transcriptional activation domain. (“AD”). In the yeast two-hybrid system, a reporter yeast strain is used that contains a specific DNA sequence, for example, a GAL4 responsive element, that can interact with a recombinant BD protein that is coupled to the bait. This responsive element is upstream of the reporter genes (for example, Lac Z and HIS3), that are regulated by this element. In addition, this responsive element is linked to a promoter sequence that can interact with a recombinant AD protein that is coupled to a library of candidate proteins. Both BD and AD domains are required for normal activation of transcription in cells or in vitro. Transcription can then be initiated by involving other components of the transcription machinery. In cells, both domains are normally part of the same protein, while in the yeast two-hybrid system, a functional activator protein can be assembled as a protein complex (BD-target protein and an AD-library protein).

Another method of identifying functionally related proteins is low stringency PCR screening of cDNA libraries with primers that are generated from functional or concerved regions of known nucleic acids or peptide or proteins.

Once the nucleic acid is identified by its interactions or homology to known sequences, translations of the nucleotide sequence of the nucleic acids, cDNAs, or full genes can be aligned with individual known sequences. This alignment may result in identification of additional, different functional domains in the identified sequence. Also, sequences exhibiting similarity with more than one individual sequence may exhibit activities that are characteristic of either or both individual sequences.

The full length sequences and fragments of the polynucleotide sequences of the nearest neighbors can be used as probes and primers to identify and isolate the full length sequence of the nucleic acid. The nearest neighbors can indicate a tissue or cell type to be used to construct a library for the full-length sequences of the nucleic acid.

Typically, the nucleic acids are translated in all six frames to determine the best alignment with the individual sequences. The sequences disclosed herein in the Sequence Listing are in a 5′ to 3′ orientation and translation in three frames can be sufficient (with a few specific exceptions as described in the Examples). These amino acid sequences are referred to, generally, as query sequences, which will be aligned with the individual sequences.

Nucleic acid sequences can be compared with known genes by any of the methods disclosed: above. Results of individual and query sequence Alignments can be divided into three categories: high similarity, weak similarity, and no similarity. Individual alignment results ranging from high similarity to weak similarity provide a basis for determining polypeptide activity and/or structure.

Parameters for categorizing individual results include: percentage of the alignment region length where the strongest alignment is found, percent sequence identity, and p value.

Percent sequence identity is calculated by counting the number of amino acid matches between the query and individual sequence and dividing total number of matches by the number of residues of the individual sequence found in the region of strongest alignment. For the example above, the percent identity would be 10 matches divided by 11 amino acids, or approximately 90.9%.

P value is the probability that the alignment was produced by chance. For a single alignment, the p value can be calculated according to Karlin et al., Proc. Natl. Acad. Sci. 87: 2264 (1990) and Karlin et al., Proc. Natl. Acad. Sci. 90: (1993). The p value of multiple alignments using the same query sequence can be calculated using an heuristic approach described in Altschul et. al., Genet. 6:119(1994). Alignment programs such as BLAST program can calculate the p value.

The boundaries of the region where the sequences align, can be determined according to Doolittle, Methods in Enzymology, supra; BLAST or FASTA programs; or by determining the area where the sequence identity is highest.

The boundaries of the region where the sequences align can be determined according to Doolittle, Methods in Enzymology, supra; BLAST or FASTA programs; or by determining the area where the sequence identity is highest.

Another factor to consider for determining identity or similarity is the location of the similarity or identity. Strong local alignment can indicate similarity even if the length of alignment is short. Sequence identity scattered throughout the length of the query sequence also can indicate a similarity between the query and profile sequences.

Probes and Primers

The nucleotide sequences determined from the cloning of nucleic acids sequences from tumor cells, especially colon cancer cell lines and tissues permit the generation of probes and primers designed for identifying and/or cloning homologs in other cell types, e.g., from other tissues, as well as homologs from other mammalian organisms. Nucleotide sequences useful as probes/primers may-include all or a portion of the sequences listed in SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or sequences complementary thereto or sequences which hybridize under stringent conditions to all or a portion of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61. For instance, the present invention also provides a probe/primer comprising a substantially purified oligonucleotide, which oligonucleotide comprising a nucleotide sequence that hybridizes under stringent conditions to at least approximately 12, preferably 25, more preferably 40, 50, or 75 consecutive nucleotides up to the full length of the sense or anti-sense sequence selected from the group consisting of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, or naturally occurring mutants thereof. For instance, primers based on a nucleic acid represented in SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46, SEQ ID NO: 48; SEQ ID NO: 50; SEQ ID NO: 51; SEQ ID NO: 52; SEQ ID NO: 60; SEQ ID NO: 61, or a sequence complementary thereto, can be used in PCR reactions to clone homoloags of that sequence.

In yet another embodiment, the invention provides probes/primers comprising a nucleotide sequence that hybridizes, under moderately stringent conditions to at least approximately 12, 16, 25, 40, 50 or 75 consecutive nucleotides up to the full length of the sense or arti-sense sequence selected from the group, consisting of SEQ ID NO 27; SEQ ID NO: 29; SEQ ID NO: 31; SEQ ID NO: 33; SEQ ID NO 35, SEQ ID NO: 37; SEQ ID NO: 38; SEQ ID NO: 40; SEQ ID NO: 42; SEQ ID NO: 44; SEQ ID NO: 46: SEQ ID NO: 48, SEQ ID NO: 50; SEQ ID NO: 51; SEQ ID NO: 52; SEQ ID NO: 60; SEQ ID NO: 61, or naturally occurring mutants thereof.

In particular, these probes are useful because they provide a method for detecting mutations in wild-type genes of the present invention. Nucleic acid probes which are complementary to a wild-type gene of the present invention and can form mismatches with mutant genes are provided, allowing for detection by enzymatic or chemical cleavage or by shifts in electrophoretic mobility. Likewise, probes based on the subject sequences can be used to detect transcripts or genomic sequences encoding the same or homologous proteins, for use, for example, in prognostic or diagnostic assays. In preferred embodiments, the probe further comprises a label group attached thereto and able to be detected, e.g., the label group is selected from radioisotopes, fluorescent compounds, chemiluminescent compounds, enzymes, and enzyme co-factors.

Full-length cDNA molecules comprising the present nucleic acids are obtained as follows. A subject nucleic acid or a portion thereof comprising at least about 12, 15, 18, or 20 nucleotides up to the full length of a sequence represented in SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, may be used as a hybridization probe to detect hybridizing members of a cDNA library using probe design methods, cloning methods, and clone selection techniques as described in U.S. Pat. No. 5,654,173, “Secreted Proteins an Polynucleotides Encoding Them,” incorporated herein by reference. Libraries of cDNA may be made from selected tissues, such as normal or tumor tissue, or from tissues of a mammal treated with, for example, a pharmaceutical agent. Preferably, the tissue is the same as that used to generate the nucleic acids, as both the nucleic acid and the cDNA represent expressed genes. Most preferably, the cDNA library is made from the biological material described here in the Examples. Alternatively,.many cDNA libraries are available commercially. (Sambrook et al., Molecular Cloning: A Laboratory Manual, 2nd Ed. (Cold Spring Harbor Press, Cold Spring Harbor, N.Y. 1989). The nucleic acid of cell type for library construction may be made after the identity of the protein encoded by the nucleic acid related gene is known. This will indicate which tissue and cell, types rare likely to express the related gene, thereby containing the mRNA for generating the cDNA.

Members of the library that are larger than the nucleic acid, and preferably that contain the whole sequence of the native message, may be obtained. To confirm that the entire cDNA has been obtained, RNA protection experiments may be performed as follows. Hybridization of a full-length cDNA to an mRNA may protect the RNA from RNase degradation. If the cDNA is not full length, then the portions of the mRNA that arc not hybridized may be subject to RNase degradation. This may be assayed, as is known in the art, by changes in electrophoretic mobility on polyacrylamide gels, or by detection of released monoribonucleotides. Sambrook et al., Molecular Cloning: A Laboratory Manual, 2nd Ed. (Cold Spring Harbor Press, Cold Spring Harbor, N.Y. 1989). In order to obtain additional sequences 5′ to the end of a partial cDNA, 5′ RACE (PCR Protocols: A Guide to Methods and Applications (Academic Press, Inc. 1990)) may be performed.

Genomic DNA may be isolated using nucleic acids in a manner similar to the isolation of full-length cDNAs. Briefly, the nucleic acids, or portions thereof, may be used as probes to libraries of genomic DNA. Preferably, the library is obtained from the cell type that was used to generate the nucleic acids. Most preferably, the genomic DNA is obtained from the biological material described herein in the Example. Such libraries may be in vectors suitable for carrying large segments of a genome, such as P1 or YAC, as described in detail in Sambrook et al., 9.4-9.36. In addition, genomic sequences can be isolated from human BAC libraries, which are commercially available from Research Genetics, Inc., Huntville, Ala., USA, for example. In order to-obtain additional 5′ or 3′ sequences, chromosome walking may be performed, as described in Sambrook et al;, such that adjacent and overlapping fragments of genomic DNA are isolated. These may be mapped and pieced together, as is known in the art, using restriction digestion enzymes and DNA ligase.

Using the nucleic acids of the invention, corresponding full length genes can benisolated using both classical and PCR methods to construct and probe cDNA libraries. Using either method, Northern blots, preferably, may be performed on a number of cell types to determine which cell lines express the gene of interest at the highest rate.

Classical methods of constructing cDNA libraries in Sambrook et al., supra. With these methods, cDNA can be produced from mRNA and inserted into viral or expression vectors. Typically, libraries of mRNA comprising poly(A) tails can be produced with poly(T) primers. Similarly, cDNA libraries can be produced using the instant sequences as primers.

PCR methods may be used to amplify the members of a cDNA library that comprise the desired insert. In this case, the desired insert may contain sequence from the full length cDNA that corresponds to the instant nucleic acids. Such PCR methods include gene trapping and RACE methods.

Gene trapping may entail inserting a member of a cDNA library into a vector. The vector then may be denatured to produce single stranded molecules. Next, a substrate-bound probe, such a biotinylated oligo, may be used to trap cDNA inserts of interest. Biotinylated probes can be linked to an avidin-bound solid substrate. PCR methods can be used to amplify the trapped cDNA. To trap sequences corresponding to the full length genes, the labeled probe sequence may be based on the nucleic acids of the invention, e.g., SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto. Random-primers or primers specific to the library vector can be used to amplify the trapped cDNA. Such gene trapping techniques are described in Gruber et al., PCT WO 95/04745 and Gruber et al., U.S. Pat. No. 5,500,356. Kits are commercially available to perform gene trapping experiments from, for example, Life Technologies, Gaithersburg, Md., USA.

“Rapid amplification of cDNA ends,” or RACE, is a PCR method of amplifying cDNAs from a number of different RNAS. The cDNAs may be ligated to an oligonucleotide, linker and amplified by PCR using two primers. One primer may be based on sequence from the instant nucleic acids, for which full length sequence is desired, and a second primer may comprise a sequence that hybridizes to the oligonucleotide linker to amplify the cDNA. A description of this method is reported in PCT, Pub. No. WO 97/19110.

In preferred embodiments, of RACE, a common primer may be designed to anneal to an arbitrary adaptor sequence ligated to cDNA ends (Apte and Siebert, Biotechniques 15:890-893, 1993; Edwards et. al., Nuc. Acids Res. 19:5227-5232, 1991). When a single gene specific RACE primer is paired with the common primer, preferential amplification of sequences between the single gene specific primer and the common primer occurs: Commercial cDNA pools modified for use in RACE are available.

Another PCR-based method generates full-length cDNA library with anchored ends without specific knowledge of the cDNA sequence. The method uses lock-docking primers (1-VI), where one primer, poly TV (I-Ill) locks over the polyA tail of eukaryotic mRNA producing first strand synthesis and a second primer, polyGH (IV-VI) locks onto the polyC tail added by terminal deoxynucleotidyl transferase (TdT). This method is described in PCT Pub. No. WO 96/40998.

The promoter region of a gene generally is located 5′ to the initiation site for RNA polymerase IL Hundreds of promoter regions contain the “TATA” box, a sequence such as TATTA or TATAA, which is sensitive to mutations. The promoter region can be obtained by performing 5′ RACE using a primer from the coding region of the gene. Alternatively, the cDNA can be used as a probe for the genomic sequence, and the region 5′ to the coding region is identified by “walking up.”

If the gene is highly expressed or differentially expressed, the promoter from the gene may be of use in a regulatory construct for a heterologous gene.

Once the full-length cDNA or gene is obtained, DNA encoding variants can be prepared by site-directed mu tagenesis, described in detail in Sambrook 15.3-15.63. The choice of codon or nucleotide to be replaced can be based on the disclosure herein on optional changes in amino acids to achieve altered protein structure and/or function.

As an alternative method to obtaining DNA or RNA from a biological material, nucleic acid comprising nucleotides having the sequence of one or more nucleic acids of the invention can be synthesized. Thus, the invention encompasses nucleic acid molecules ranging in length from 12 nucleotides (corresponding to at least 12 contiguous nucleotides which hybridize under stringent conditions to or are at least 80% identical to a nucleic acid represented by one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO; 61, or a sequence complementary thereto) up to a maximum length suitable for one or more biological manipulations, including replication and expression, of the nucleic acid molecule. The invention includes but is not limited to (a) nucleic acid having the size of a full gene, and comprising at least one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto; (b) the nucleic acid of(a) also comprising at least one additional gene, operably linked to permit expression of a fusion protein; (c) an expression vector comprising (a) or (b); (d) a plasmid comprising (a) or (b); and (e) a recombinant viral particle comprising (a) or (b). Construction of (a) can be accomplished as described below (Vectors Carrying Nucleic Acid of the Present Invention).

The sequence of a nucleic acid of the present invention is not limited and can be any sequence of A, T, G, and/or C (for DNA) and A, U, G, and/or C (for RNA) or modified bases thereof, including inosine and pseudouridine. The choice of sequence will depend on the desired function and can be dictated by coding regions desired, the intron-like regions desired, and the regulatory regions desired.

Vectors Carrying Nucleic Acids of the Present Invention

The invention further provides plasmids and vectors, which can be used to express a gene in a host cell. The host cell maybe any prokaryotic or-eukaryotic cell; Thus, a nucleotide sequence derived from any one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, encoding all or a selected portion of a protein, can be used to produce a recombinant form of an polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures well known in the art.

Vectors that allow expression of a nucleic acid in a cell are referred to as expression vectors. Typically, expression vectors contain a nucleic acid operably linked to at least one transcriptional regulatory sequence. Regulatory sequences are art-recognized and are selected to direct expression of the subject nucleic acids. Transcriptional regulatory sequences are described in Goeddel; Gene Expression Technology: Methods in Enzymology 185, Academic Press, San Diego, Calif. (1990). In one embodiment, the expression vector includes a recombinant gene encoding a peptide having an agonistic activity of a subject polypeptide, or alternatively, encoding a peptide which is an antagonistic form of a subject polypeptide.

The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. The nucleic acid or full-length gene is inserted into a vector typically by means of DNA ligase attachment to a cleaved restriction enzyme site in the vector. Alternatively, the desired nucleotide sequence may be inserted by homologous recombination in vivo. Typically this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence.

Nucleic acids or full-length genes are linked to regulatory sequences, as appropriate to obtain the desired expression properties. These may include promoters (attached either at the 5′ end of the sense strand or at the 3′ end of the antisense strand), enhancers, terminators, operators, repressors and inducers. The promoters may be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art may be used.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Once the gene corresponding to the nucleic acid is identified, its expression can be regulated in the cell to which the gene is native. For example, an endogenous gene of a cell can be regulated by an exogenous regulatory sequence as disclosed in U.S. Pat. No. 5,641,670, “Protein Production and Protein Delivery.”

A number of vectors exist for the expression of recombinant proteins in yeast (see, for example, Broach et al (1983) in Experimental Manipulation of Gene Expression, ed. M. Inouye, Academic Press, p. 83, incorporated by reference herein). In addition, drug resistance markers such as ampicillin can be used. In an illustrative embodiment, a polypeptide is produced recombinantly utilizing an expression vector generated by sub-cloning one of the nucleic acids represented in one of SEQ ID Nos. 1-544, preferably SEQ ID Nos. 1-168, even more preferably SEQ ID Nos. 1-35, or a sequence complementary thereto.

The preferred mammalian expression vectors contain both prokaryotic sequences, to facilitate the propagation of the vector in bacteria, and one or more eukaryotic transcription units that are expressed in eukaryotic cells. The various methods employed in the preparation of plasmids and transformation of host organisms are well known in the art. For other suitable expression systems for both prokaryotic and eukaryotic cells, as well as general recombinant procedures, see Molecular-Cloning: A Laboratory Manual, 2nd Ed., ed. by Sambrook, Fritsch and Maniatis (Cold Spring Harbor Laboratory Press: 1989) Chapters 16 and 17.

When it is desirable to express only a portion of a gene, e.g., a truncation mutant, it may be necessary to add a start codon (ATG) to the oligonucleotide-fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine, if desired, can be achieved either in vivo by expressing polypeptides in a host which produces MAP (e.g., E. coli or CM89 or S. cerevisiae), or in vitro by use of purified MAP (e.g., procedure of Miller et al., supra).

Moreover, the nucleic acid constructs of the present invention can also be used as part of a gene therapy protocol to deliver nucleic acids such as antisense nucleic acids. Thus, another aspect of the invention features expression vectors for in vivo or in vitro transfection with an antisense oligonucleotide.

In addition to viral transfer methods, non-viral methods can also be employed to introduce a subject nucleic acid, e.g., a sequence represented by one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, into the tissue of an animal. Most nonviral methods of gene transfer rely on normal mechanisms used by mammalian cells for the uptake and intracellular transport of macromolecules. In preferred embodiments, non-viral targeting means of the present invention rely on endocytic pathways for the uptake of the subject nucleic acid by the targeted cell. Exemplary targeting means of this type include liposomal derived systems, polylysine conjugates, and artificial viral envelopes.

A nucleic acid of any of SEQ ID NO: 27; SEQ ID NO: 29; SEQ ID NO:,3 1; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, the corresponding cDNA, or the full-length gene may-be used to express the partial or complete gene product. Appropriate nucleic acid constructs are purified using standard recombinant DNA techniques as described in, for example, Sambrook et al., (1989) Molecular Cloning: A Laboratory Manual, 2nd ed. (Cold Spring Harbor Press, Cold Spring Harbor, N.Y.), and under current regulations described in United States Dept. of HHS, National Institute of Health (NH) Guidelines for Recombinant DNA Research. The polypeptides encoded by the nucleic acids may be expressed in any expression system, including, for example, bacterial, yeast, insect, amphibian and mammalian systems. Suitable vectors and host cells are described, e.g., in U.S. Pat. No. 5,654,173.

Bacteria. Expression systems in bacteria include those described in Chang et al., Nature (1978) 275:615, Goeddel et al., Nature (1979) 281 :544, Goeddel et al., Nucleic Acids Rec. (1980) 8:4057; EP 0 036,776, U.S. Pat. No. 4,551,433, DeBoer et al., Proc. Natl. Acad. Sci. (USA) (1983) 80:2125, and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (19 85) 25:141; Gleeson et al., J. Gen. Microbiol. (1986) 132:3459, Roggenkamp et al., Mol. Gen. Genet. (1986) 202:302) Das et al., J. Bacteriol. (1984) 158:1165; De Louvencourt et al., J. Bacteriol. (1983) 154:737, Van den Berg et al., Bio/Technology (1990) 8:135; Kunze et al., J. Basic Microbiol. (1985) 25:141; Cregg et al., Mol. Cell. Biol. (1985) 5:3376, U.S. Pat. Nos. 4,837,148 and 4,929,555; Beach and Nurse, Nature (1981) 300:706; Davidow et al., Curr. Genet. (1985) 10:380, Gaillardin et al., Curr., Genet. (1985) 10:49, Ballance et al., Biochem. Biophys. Res. Commun. (1983) 112:284289; Tilburn et al., Gene (1983) 26:205221, Yelton et al., Proc. Natl. Acad. Sci. (USA) (1984) 81:14701474, Kelly and Hynes, EMBO J. (1985) 4:475479; EP 0 244,234, and WO 91/00357.

Insect Cells. Expression of heterologous genes in insects is accomplished-as-described in U.S. Pat. No. 4,745,051, Friesen et al., (1986) “The Regulation of Baculovirus Gene Expression” in: The Molecular Biology Of Baculoviruses (W. Doerfler, ed.), EP 0 127,839, EP 0 155,476, and Vlak et al., J., Gen. Virol. (1988) 69:765776, Miller et al., Ann. Rev. Microbiol. (1988) 42:177, Carbonell et al., Gene (1988) 73:409, Maeda et al., Nature (1985) 315:592594, Lebacq Verheyden et al, Mol. Cell. Biol. (1988) 8:3129; Smith et al., Proc. Nail. Acad. Sci. (USA) (1985) 82:8404, Miyajima et al., Gene (1987) 58:273; and Martinet al., DNA (1988) 7:99. Numerous baculoviral strains and variants and corresponding permissive insect host cells from hosts are described in Luckow et al., Bio/Technology (1988) 6:4755, Miller et al., Generic Engineering (Setlow, J. K. et al. eds.), Vol. 8 (Plenum Publishing, 1986), pp.277279, and Maeda et al., Nature, (1:985) 315:592-594.

Mammalian Cells. Mammalian expression is accomplished as described in Dijkema et al., EMBO J. (1985) 4:76.1, Gorman et al., Proc. Natl. Acad. Sci. (USA) (1982) 79:6777, Boshart et al., Cell (1985)41-52 1 and U.S. Pat. No. 4,399,216. Other features of mammalian expression are facilitated as described in Hain and Wallace, Meth. Enz. (1979) 58:44, Barnes and Sato, Anal. Biochem. (1980) 102:255, U.S. Pat. Nos. 4,767,704, 4,657,866, 4,927,762; 4,560,655, WO 90/103430, WO 87/00195, and U.S. RE 30,985.

Therapeutic Nucleic Acid Constructs

One aspect of the invention relates to the use of the isolated nucleic acid, e.g., SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, in antisense therapy. As used herein, antisense therapy refers to administration or in situ generation of oligonucleotide molecules or their derivatives which specifically hybridize (e.g., bind) under cellular conditions with the cellular mRNA and/or genomic DNA, thereby inhibiting transcription and/or translation of that gene. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA. Alternatively, the antisense construct is an oligonucleotide probe which is generated ex vivo and which, when introduced into the cell, causes inhibition of expression by hybridizing with the mRNA and/or genomic sequences of a subject nucleic acid. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphorothlioate and methylphosphonate analogs of DNA (see also, U.S. Pat. Nos. 5,176,996; 5,264,564; and 5,2:56;775). Additionally, general approaches to constructing oligomers useful in antisense therapy have been reviewed, for example, by Van der Kaol et al. (1988) BioTechniques 6:958-976; and Stein et. al. (1988) Cancer Res 48:2659-2668. With respect to antisense DNA, oligodeoxyribonucleotides derived from the translation initiation site, e.g., between the −10 and +10 regions of the nucleotide sequence of interest, are preferred.

Antisense approaches involve the design of oligonucleotides (either DNA or RNA) that are complementary to mRNA. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are typically less efficient inhibitors of translation but could also-be used in accordance with the invention. Whether designed to hybridize to the 5′, 3′, or coding region of subject mRNA, antisense nucleic acids should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense oligonucleotide to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO 88/098 10, published Dec. 15, 1988) or the blood-brain barrier (see, e.g., PCT Publication No. WO 89/10 134, published Apr. 25, 1988), hybridization-triggered cleavage agents (See, e.g., Krol et al., 1988, BioTechniques 6:958-976), or intercalating agents (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytriethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v}, 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Peny-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methyiphosphonate, an alkyl phosphotriester, and a formacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual n-units, the strands run parallel to each other (Gautier et al, 1987, Nucl. Acids Res. 15:6625-6641). The oligonucleotide is a 2′-O-methylribonucleotide (Iqoue et al., 1987, Nucl. Acids Res. 15:6131-12148), or a chimeric RNA-DNA analogue (Jnoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate olgonucleotides can be prepared by use of controlled pore glass, polymer supports (Sarin et al.,. 1988, Proc. Natl. Acad. Sci. U.S.A. 85:7448-7451), etc.

While antisense nucleotides complementary to a coding region sequence can be used, those complementary to the transcribed untranslated region and to the region comprising the initiating methionine are most preferred.

The antisense molecules can be delivered to cells which express the target nucleic acid in vivo. A number of methods have been developed for delivering antisense DNA of RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation on endogenous mRNAs. Therefore, a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous,transcripts and thereby prevent translation of the target mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-3 10), the promoter contained in the 3′ long terminal repeat of Rous sarcoma virus (Yamamoto et al., 1980, Cell 22:787-797), the herpes thymidine kinase promoter (Wagner et ail., 1981, Proc. Natl. Acad. Sci. U.S.A. 78:1441-1445), the regulatory sequences of the metallothionein gene (Brinster et at, 1982, Nature 296:39-42), etc. Any type of plasmid, cosmid, YAC or viral vector can be used to prepare the recombinant DNA construct which can be introduced directly into the tissue site; e.g., the choroid plexus or hypothalamus. Alternatively, viral vectors can be used which selectively infect the desired tissue (e.g., for brain, herpesvirus vectors may be used), in which case administration may be accomplished by another route (e.g., systemically).

In another aspect of the invention, ribozyme molecules designed to catalytically cleave target mRNA transcripts can be used to prevent translation of target mRNA and expression of a target protein (See, e.g., PCT International Publication WO90/11364, published Oct. 4, 1990; Sarver et al., 1990, Science 247:1222-1225 and U.S. Pat. No. 5,093,246). While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the target mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the TVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. W088/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene.

As in the antisense approach, the ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous messages and inhibit translation. Because ribozymes, unlike antisense molecules, are catalytic, a lower intracellular concentration is required for efficiency.

Antisense RNA, DNA, and ribozyme molecules of the invention may be prepared by any method known in the art for the synthesis of DNA and RNA molecules. These include techniques for chemically synthesizing oligodeoxyribonucleotides and oligoribonucleotides well known in the art such as- for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′0-methyl rather than phosphodiesterase linkages within the oligodeoxyribonucleotide backbone.

Polypeptides of the Present Invention

The present invention makes available isolated polypeptides which are isolated from, or otherwise substantially free of other cellular proteins, especially other signal transduction factors and/or transcription factors which may normally be associated with the polypeptide. Subject polypeptides of the present invention include polypeptides encoded by the nucleic acids of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO0 60; SEQ ID NO: 61, or a sequence complementary thereto, or polypeptides encoded by genes of which a sequence in SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, is a fragment. Polypeptides of the present invention include those proteins which are differentially regulated in tumor cells, especially colon cancer-derived cell lines (relative to normal cells, e.g., normal colon tissue and non-colon tissue). In preferred embodiments, the polypeptides are upregulated in tumor cells, especially colon cancer cancer-derived cell lines. In other embodiments, the polypeptides are downregulated in tumor cells, especially colon cancer-derived cell lines. Proteins which are upregulated, such as oncogenes, or downregulated, such as tumor suppressors, in aberrantly proliferating cells may be targets for diagnostic or therapeutic techniques.

The term “substantially free of other cellular proteins” (also referred to herein as “contaminating proteins”) or “substantially pure or purified preparations” are defined as encompassing preparations of polypeptides having less than about 20% (by dry weight) contaminating protein, and preferably having less than about 5% contaminating protein. Functional forms of the subject polypeptides can be prepared, for the first time, as purified preparations by using a cloned nucleic acid as described herein. Full length proteins or fragments corresponding to one or more particular motifs and/or domains or to arbitrary sizes, for example, at least about 5, 10, 25, 50, 75, or 100 amino acids in length are within the scope of the present invention.

For example, isolated polypeptides can be encoded by all or a portion of a nucleic acid sequence shown in any of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto. Isolated peptidyl portions of proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) protein.

Another aspect of the present invention concerns recombinant forms of the subject proteins. Recombinant polypeptides preferred by the present invention, in addition to native proteins, as described above are encoded by a nucleic acid, which is at least 60%, more preferably at least 80%, and more preferably 85%, and more preferably 90%, and more preferably 95% identical to an amino acid sequence encoded by SEQ ID Nos. 1-544. Polypeptides which are encoded by a nucleic acid that is at least about 98-100% identical with the sequence of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 are also within the scope of the invention. Also included in the present invention are peptide fragments comprising at least a portion of such a protein.

In a preferred embodiment, a polypeptide of the present invention is a mammalian polypeptide and even more preferably a human polypeptide. In particularly preferred embodiment, the polypeptide retains wild-type-bioactivity. It will be understood that certain post-translational modifications, e.g., phosphorylation and the like, can increase the apparent molecular weight of the polypeptide relative to the unmodified polypeptide chain.

The present invention further pertains to recombinant forms of one of the subject polypeptides. Such recombinant polypeptides preferably are capable of functioning in one of either role of antagonist or antagonist of at least one biological activity of a wild-type (“authentic”) polypeptide of the appended sequence listing. The term “evolutionarily related to”, with respect to amino acid sequences of proteins, refers to both polyp eptides having amino acid sequences which have arisen naturally, and also to mutational variants of human polypeptides which are derived, for example, by combinatorial-mutagenesis.

In general, polypeptides referred to herein as having an activity (e.g., are “bioactive”) of a protein are defined as polypeptides which include an amino acid sequence encoded by all or a portion of the nucleic acid sequences shown in one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID:NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, and which mimic or antagonize all or a portion of the biological/biochemical activities of a naturally occurring protein. According to the present invention, a polypeptide has biological activity if it is a specific agonist or antagonist of a naturally occurring form of a protein.

Assays for determining whether a compound, e.g, a protein or variant thereof, has one or more of the above biological activities are well known in the art. In certain embodiments, the polypeptides of the present invention have activities such as those outlined above.

In another embodiment, the coding sequences for the polypeptide can be incorporated as a part of a fusion gene including a nucleotide sequence encoding a different polypeptide. This type of expression system can be useful under conditions where it is desirable to produce an immunogenic fragment of a polypeptide (see, for example, EP Publication No: 0259149; and Evans et al. (1989) Nature 339:3 85; Huang et at. (1988) J. Virol. 62:3 855; and Schlienger et al., (1992) J. Virol. 66:2). In addition to utilizing fusion proteins to enhance immunogenicity, it is widely appreciated that fusion proteins can also facilitate the expression of proteins, and, accordingly, can be used in the expression of the polypeptides of the present invention (see, for example, Current Protocols in Molecular Biology, eds. Ausubel et at. (N.Y. John Wiley & Sons, 1991)). In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified protein (e.g., see Hochuli et al. (1987)J. Chromatography 411:177; and Janknecht et al. PNAS 88-8972)..

Techniques for making fusion genes are known to those skilled in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene, can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed to generate a chimeric nucleic acid sequence (see, for example, Current Protocols in Molecular Biology, eds. Ausubel et al. John Wiley & Sons: 1992).

The present invention further pertains to methods of producing the subject polypeptides. For example, a host cell transfacted with a nucleic acid vector directing expression of a nucleotide sequence encoding the subject polypeptides can be cultured under appropriate conditions to allow expression of the peptide to occur. Suitable media for cell culture are well known in the art. The recombinant polypeptide can be isolated from cell culture medium, host cells, or both using techniques known in the art for purifying proteins including ion-exchange chromatography, gel filtration chromatography, ultrafiltration, electrophoresis, and immnunoaffinity purification with antibodies specific for such peptide. In a preferred embodiment, the recombinant polypeptide is a fusion protein containing a domain which facilitates its purification, such as GST fusion protein.

Moreover, it will be generally appreciated that, under certain circumstances, it may be advantageous to provide homologs of one of the subject polypeptides which function in a limited capacity as one of either an agonist (mimetic) or an antagonist, in order to promote or inhibit only a subset of the biological activities of the naturally occurring form of the protein. Thus, specific biological effects can be elicited by treatment with a homolog of limited function, and with fewer side effects relative to treatment with agonists or antagonists which are directed to all of the biological-activities of naturally occurring forms of subject proteins.

Homologs of each of the subject polypeptide can be generated by mutagenesis, such as by discrete point mutation(s), or by truncation. For instance, mutation can give rise to homologs which retain substantially the same, or merely a subset, of the biological activity of the polypeptide from which it was derived. Alternatively, antagonistic forms of the polypeptide can be generated which are able to inhibit the function of the naturally occurring form of the protein, such as by competitively binding to a receptor.

The recombinant polypeptides of the present invention also include homologs of the wild-type proteins, such as versions of those proteins which are resistant to proteolytic cleavage, for example, due to mutations which alter ubiquitination or other enzymatic targeting associated with the protein.

Polypeptides may also be chemically modified to create derivatives by forming covalent or aggregate conjugates with other chemical moieties; such as glycosyl groups, lipids, phosphate, acetyl groups and the like. Covalent derivatives of proteins can be prepared by linking the chemical moieties to functional groups on amino acid sidechains of the protein or at the N-terminus or at the C-terminus of the polypeptide.

Modification of the structure of the subject polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally occurring form of the protein, or to produce specific antagonists thereof, are considered functional equivalents of the polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e., isosteric and/or isoelectric mutations) will not have a major effect on the biological-activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alantine, valine, leucirie, isoleucine, serine, threonine, with serine and threonine optionally be grouped separately as aliphatic-hydroxyl; (4) aromatic=phenylalanine, tyrosine, tiyptophan; (5) amide=asparagine, glutamine; and (6) sulfur-containing=cysteine and methionine. (see, for example, Biochemistry, 2 ed., Ed. by L. Stryer, WH Freeman and Co.: 1981). Whether a change in the amino acid-sequence of a peptide results in a functional homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response.

Polypeptides in which more than one replacement has -taken place can readily be tested in the same manner. The variant may be designed so as to retain biological activity of a particular region of the protein. In a non-limiting example, Osawa et al., 1994, Biochemistry and Molecular International 34:1003-1009, discusses the actin binding region of a protein from several different species. The actin binding regions of the these species are considered homologous based on the fact that they have amino acids that fall within “homologous residue groups.” Homologous residues are judged according to the following groups (using single letter amino acid designations): STAG; ILVMF; SRK; DEQN; and FYW. For example, an S, a T, an A or a G can be in a position and the function (in- this case actin binding) is retained.

Additional guidance on amino acid substitution is available from studies of protein evolution. Go et al., 1980, Int. J. Peptide Protein Res. 15: 211-224, classified amino acid residue sites as interior or exterior depending on their accessibility. More frequent substitution on exterior sites was confirmed to be general in eight sets of homologous protein families regardless of their biological-functions and the presence or absence of a prosthetic group. Virtually all types of amino acid residues had higher mutabilities on the exterior than in the interior. No correlation between mutability and polarity was observed of amino acid residues in the interior and exterior, respectively. Amino acid residues were classified into one of three groups depending on their polarity: polar (Arg, Lys, His, Gln, Asn, Asp, and Glu); weak polar (Ala, Pro, Gly, Thr, and Ser), and nonpolar (Cys, Val, Met, Ile, Leu, Phe, Tyr, and Trp). Amino acid replacements during protein evolution were very conservative: 88% and 76% of them in the interior or exterior, respectively, were within the same group of the three. Intergroup replacements are such that weak polar residues are replaced more often by nonpolar residues in the interior and more often by polar residues on the exterior.

Diagnostic and Prognostic Assays and Drug Screening Methods

The present invention provides method for determining whether a subject is at risk for developing a disease or condition characterized by unwanted cell proliferation by detecting the disclosed biomarkers, i.e., the disclosed nucleic acid markers (SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61) and/or polypeptide markers for colon cancer encoded thereby.

In clinical applications, human tissue samples can be screened for the presence and/or absence of the biomarkers identified herein. Such samples could consist of needle biopsy cores, surgical resection samples, lymph node tissue, or serum. For example, these methods include obtaining a biopsy, which is optionally fractionated by cryostat sectioning to enrich tumor cells to about 80% of the total cell population. In certain embodiments, nucleic acids extracted from these samples may be amplified using techniques well known in the art. The levels of selected markers detected would be compared with statistically valid groups of metastatic, non-metastatic malignant, benign, or normal colon tissue samples.

In one embodiment, the diagnostic method comprises determining whether a subject has an abnormal mRNA and/or protein level of the disclosed markers, such as by Northern blot analysis, reverse transcription-polymerase chain reaction (RT-PCR), in situ hybridization, immunoprecipitation, Western blot hybridization, or immunohistodhenistry. According to the method, cells are obtained from a subject and the levels of the disclosed biomarkers, protein or mRNA level, is determined and compared to the level of these markers in a healthy subject. An abnormal level of the biomarker polypeptide or mRNA levels is likely to be indicative of cancer such as colon cancer.

Accordingly, in one aspect, the invention provides probes arid primers that are specific to the unique nucleic acid markers disclosed herein. Accordingly, the nucleic acid probes comprise a nucleotide sequence at least 12 nucleotides in length, preferably at least 15 nucleotides, more preferably, 25 nucleotides, and most preferably at, least 40 nucleotides, and up to all or nearly all of the coding sequence which is complementary to a portion of the coding sequence of a marker nucleic acid sequence, which nucleic acid sequence is represented by SEQ ID NO: 27; SEQ ID NO: 29; SEQ ID NO: 31; SEQ ID NO: 33; SEQ ID NO: 35; SEQ D NO: 37; SEQ ID NO: 38; SEQ ID NO:40; SEQ ID NO:42; SEQ ID NO:44; SEQ ID NO: 46;SEQ ID NO:48; SEQ ID NO: 50; SEQ ID NO: 51; SEQ ID NO: 52; SEQ ID NO: 60; SEQ ID NO: 61 or a sequence complementary thereto.

In one embodiment, the method comprises using a nucleic acid probe to determine the presence of cancerous cells in a tissue from a patient. Specifically, the method comprises:
1. providing a nucleic acid probe comprising a nucleotide sequence at least 12 nucleotides in length, preferably at least 15 nucleotides, more preferably, 25 nucleotides, and most preferably at least 40 nucleotides, and up to all or nearly all of the coding sequence which is complementary to a portion of the coding sequence of a nucleic acid sequence represented by SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ I NO: 46; SEQ ID NO: 48; SEQ ID NO: 50; SEQ ID NO: 51; SEQ ID NO: 52; SEQ ID NO: 60; SEQ ID NO: 61 or a sequence complementary thereto and is differentially expressed in tumors cells, such as colon cancer cells;
2. obtaining a tissue sample from a patient potentially comprising cancerous cells; providing a second tissue sample containing cells substantially all of which are non-cancerous;
4. contacting the nucleic acid probe under stringent conditions with RNA of each of said first and second tissue samples (e.g., in a Northern blot or in situ hybridization assay); and
5. comparing (a) the amount of hybridization of the probe with RNA of the first tissue sample, with (b) the amount of hybridization of the probe with RNA of the second tissue sample; wherein a statistically significant difference in the amount of hybridization with the RNA of the first tissue sample as compared to the amount of hybridization with the RNA of the second tissue sample is indicative of the presence of cancerous cells in the first tissue sample.

In one aspect, the method comprises in situ hybridization with a probe derived from a given marker nucleic acid sequence, which nucleic acid sequence is represented by SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ D NO: 51; SEQ ID NO: 52; SEQ ID NO: 60; SEQ ID NO: 61 or a sequence complementary thereto. The method comprises contacting the labeled hybridization probe with a sample of a given type of tissue potentially containing cancerous or pre-cancerous cells as well as normal cells, and determining whether the probe labels some cells of the given tissue type to a degree significantly different (e.g., by at least a factor of two, or at least a factor of five, or at least a factor of twenty, or at least a factor of fifty) than the degree to which it labels other cells of the same tissue type.

Also within the invention is a method of determining the phenotype of a test cell from a given human tissue, e.g., whether the cell is (a) normal, or (b) cancerous or precancerous, by contacting the mRNA of a test cell with a nucleic acid probe at least 12 nucleotides in length, preferably at least 15 nucleotides, more preferably at least 25 nucleotides, and most preferably at least 40 nucleotides, and up to all or nearly all of a sequence which-is complementary to a portion of the coding sequence of a nucleic acid sequence represented by SEQ ID NO: 27; SEQ ID NO: 29; SEQ ID NO: 31; SEQ ID NO: 33; SEQ ID NO: 35; SEQ; ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto, and which is differentially expressed in tumor cells as compared to normal cells of the given tissue type; and determining the approximate amount of hybridization of the probe to the mRNA, an amount of hybridization either more or less than that seen with the mRNA of a normal cell of that tissue type being indicative that the test cell is cancerous or pre-cancerous.

Alternatively, the above diagnostic assays may be carried out using antibodies to detect the protein product encoded by the marker nucleic acid sequence, which nucleic acid sequence is represented by SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto. Accordingly, in one embodiment, the assay would include contacting the proteins of the test cell with an antibody specific for the gene product of a nucleic acid represented by SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or a sequence complementary thereto, the marker nucleic acid being one which is expressed at a given control level in normal cells of the same tissue type as the test cell, and determining the approximate amount of immunocomplex formation by the antibody and the proteins of the test cell, wherein a statistically significant difference in the amount of the immunocomplex formed with the proteins of a test cell as compared to a normal cell of the same tissue type is an indication that the test cell is cancerous or pre-cancerous.

Another such method includes the steps of: providing an antibody specific for the gene product of a marker nucleic acid sequence represented by SEQ ID NO: 27; SEQ ID NO: 29; SEQ D NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, the gene product being present in cancerous tissue of a given tissue-type (e.g., colon tissue) at a level more or less than the level of the gene product in non-cancerous tissue of the same tissue -type; obtaining from a patient a first sample of.,tissue of the given tissue type, which sample potentially includes cancerous cells; providing a second sample of tissue of the same tissue type (which may be from the same patient or from a normal control, e.g. another individual or cultured cells), this second sample containing normal cells arid essentially no cancerous cells; contacting the antibody with protein (which may be partially purified, inlysed but unfractionated cells, or in situ) of the first and second samples under conditions permitting immunocomplex formation between the antibody and the marker nucleic acid sequence product present in the samples; and comparing (a) the amount of immunocomplex formation-in the first sample, with (b) the amount of immunocomplex formation in the second sample, wherein a statistically significant difference in the amount of immunocomplex formation in the first sample less as compared to the amount of immunocomplex formation in the second sample is indicative of the presence of cancerous cells in the first sample of tissue.

The subject invention further provides-a method of determining whether a cell sample obtained from a subject possesses an abnormal amount of marker polypeptide which comprises (a) obtaining a cell sample from the subject, (b) quantitatively determining the amount of the marker polypeptide in the sample so obtained, and (c) comparing the amount of the marker polypeptide so determined with a known standard, so as to thereby determine whether the cell sample obtained from the subject possesses an abnormal amount of the marker polypeptide. Such marker polypeptides may be detected by immunohistochemical assays, dot-blot assays, ELISA and the like.

Immunoassays are commonly used to quantitate the levels of proteins in cell samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarization immunoassay (FPIA), fluorescence immunoassay (FIA), enzyme immunoassay (EIA) nephelometric inhibition immunoassay (NLA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

In another embodiment, the level of the encoded product, i.e., the product encoded by SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ II) NO: 44; SEQ ID) NO: 46; SEQ ID NO: 48; SEQ ID NO: 50; SEQ ID NO: 51; SEQ ID NO: 52; SEQ ID NO: 60; SEQ ID NO: 61 or a sequence complementary thereto, in a biological fluid (e.g., blood or urine) of a patient maybe determined as a way of monitoring the level of expression of the marker nucleic acid sequence in cells of that patient. Such a method would include the steps of obtaining a sample of a biological fluid from the patient, contacting the sample.(or proteins from the sample) with an antibody specific for a encoded marker polypeptide, and determining the amount of immune complex formation by the antibody, with the amount of immune complex formation being indicative of the level of the marker encoded product in the sample. This determination is particularly instructive when compared to the amount of immune complex formation by the same antibody in a control sample taken from a normal individual or in one or more, samples previously or subsequently obtained from the same person.

In another embodiment, the method can be used to determine the amount of marker polypeptide present in a cell, which in turn can be correlated with progression of a hyperproliferative disorder, e.g., colon cancer. The level of the marker polypeptide can be used predictively to evaluate whether a sample of cells contains cells which are, or are predisposed towards becoming, transformed cells. Moreover, the subject method can be used to assess the phenotype of cells which are known to be transformed, the phenotyping results being useful in planning a particular therapeutic regimen. For instance, very high levels of the marker polypeptide in sample cells is a powerful diagnostic and prognostic marker for a cancer, such as colon cancer: The observation of marker polypeptide level can be utilized in decisions regarding, e.g., the use of more aggressive therapies.

As set out above, one aspect of the present invention relates to diagnostic assays for determining, in the context of cells isolated from a patient, if the level of a marker polypeptide is significantly reduced in the sample cells. The term “significantly reduced” refers to a cell phenotype wherein the cell possesses a reduced cellular amount of the marker polypeptide relative to a normal cell of similar tissue origin. For example, a cell may have less than; about 50%, 25%, 10%, or 5% of the marker polypeptide that a normal control cell. In particular, the assay evaluates the level of marker polypeptide in the test cells, and preferably, compares the measured level with marker polypeptide detected in at least one.control cell, e.g., a normal cell and/or a transformed cell of known phenotype.

Of particular importance to the subject invention is the ability to quantitate the level of marker polypeptide as determined by the number of cells associated with a normal or abnormal marker polypeptide level. The number of cells with a particular marker polypeptide phenotype may then be correlated with patient prognosis. In one embodiment of the invention, the marker polypeptide phenotype of the lesion is determined as a percentage of cells in a biopsy which are found to have abnormally high/low levels of the marker polypeptide. Such expression may be detected by immunohistochemical assays, dot-blot assays, ELISA and the like.

Where tissue samples are employed, immunohistochemical staining may be used to determine the number of cells-having the marker polypeptide phenotype. For such staining, a multiblock of tissue is taken from the biopsy or other tissue sample and subjected to profeolytic hydrolysis, employing such agents as protease K or pepsin. In certain embodiments, it may be desirable to isolate a nuclear fraction from the sample cells and detect the level of the marker polypeptide in the nuclear fraction.

The tissue samples are fixed by treatment with a reagent such as formalin, glutaraldehyde, methanol, or the like. The samples are then incubated with an antibody, preferably a monoclonal antibody, with binding specificity for the marker polypeptides. This antibody may be conjugated to a label for subsequent detection of binding. Samples are incubated for a time sufficient for formation of the immunocomplexes. Binding of the antibody is then detected by virtue of a label conjugated to this antibody. Where the antibody is unlabeled, a second labeled antibody may be employed, e.g., which is specific for the isotype of the anti-marker polypeptide antibody. Examples of labels which may be employed include radionuclides, fluorescers, chemiluniinescers, enzymes and the like.

Where enzymes are employed, the substrate for the enzyme may be added to the samples to provide a colored or fluorescent product. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

In one embodiment, the assay is performed as a dot blot assay. The dot blot assay finds particular application where tissue samples are employed as it allows determination Of the average amount of the marker polypeptide associated with a single cell-by correlating the amount of marker polypeptide in a cell-free extract produced from a:predetermined number of cells.

It is well established in the cancer literature that tumor cells of the same type (e.g., breast and/or colon tumor cells) may not show uniformly increased expression of individual oncogenes or uniformly decreased expression of individual tumor suppressor genes. There may also be varying levels of expression of a given marker gene even between cells of a given type of cancer, further emphasizing the need for reliance on a battery of tests rather than a single test. Accordingly, in one aspect, the invention provides for a battery of tests utilizing a number of probes of the invention, in order to improve the reliability and/or accuracy of the diagnostic test.

In one embodiment, the present invention also provides a method wherein nucleic acid probes are immobilized on a DNA chip in an organized array. Oligonucleotides can be bound to a solid support by a variety of processes, including lithography. For example a chip can hold up to 250,000 oligonucleotides (e.g., GeneChip® applications available from Affymetrix, Santa Clara, Calif.). These nucleic acid probes comprise a nucleotide sequence at least about 12 nucleotides in length, preferably at least about 15 nucleotides, more preferably at least about 25 nucleotides, and most preferably at least about 40 nucleotides, and up to all or nearly all of a sequence which is complementary to a portion of the coding sequence of a marker nucleic acid sequence represented by SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 and is differentially expressed in tumor cells, such as colon cancer cells. The present invention provides significant advantages over the available tests for various cancers, such as colon cancer, because it increases the reliability of the test by providing an array of nucleic acid markers on a single chip.

The method includes obtaining abiopsy, which is optionally fractionated by cryostat sectioning to enrich tumor cells to about 80% of the total cell population. The DNA or RNA is then extracted, amplified, and analyzed with a DNA chip to determine the presence of absence of the marker nucleic acid sequences.

In one embodiment, the nucleic acid probes are spotted onto a substrate in a two-dimensional matrix or array. Samples of nucleic acids can be labeled and then hybridized to the probes. Double-stranded-nucleic acids, comprising the labeled sample nucleic acids bound to probe nucleic acids, can be detected once the unbound portion of the sample is washed away.

The probe nucleic acids can be spotted on substrates including glass, nitrocellulose, etc. The probes can be bound to the substrate by either covalent bonds or by non-specific interactions, such as hydrophobic interactions. The sample nucleic acids can be labeled using radioactive labels, fluorophores, chromophores, etc.

Techniques for constructing arrays and methods of using these arrays are described in EP No. 0 799 897; PCT No. WO 97/292 12; PCT No. WO 97127317; EP No. 0 785 280; PCT No. WO 97/02357; U.S. Pat. No. 5,593,839; U.S. Pat. No. 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734.

Further, arrays can be used to examine differential expression of genes and can be used to determine gene function. For example, arrays of the instant nucleic acid sequences can be used to determine if any of the nucleic acid sequences are differentially expressed between normal cells and cancer cells, for example. High expression of a particular message in a cancer cell, which is not observed in a corresponding normal cell, can indicate a cancer specific protein.

In yet another embodiment, the invention contemplates using a panel of antibodies which are generated against the marker polypeptides of this invention, which polypeptides are encoded by SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61. Such a panel of antibodies may be used as a reliable diagnostic probe for colon cancer. The assay of the present invention comprises, contacting a biopsy sample containing cells, e.g., colon cells, with a panel of antibodies to one or more of the encoded products to determine the presence or absence of the marker polypeptides.

The diagnostic methods of the subject invention may also be employed as follow-up to treatment, e.g., quantitation of the level of marker polypeptides may be indicative of the effectiveness of current or previously employed cancer therapies as well as the effect of these therapies upon patient prognosis.

Accordingly, the present invention makes available diagnostic assays and reagents for detecting gain and/or loss of marker polypeptides from a cell in order to aid in the diagnosis and phenotyping of proliferative disorders arising from, for example, tumorigenic transformation of cells.

The diagnostic assays described above can be adapted to be used as prognostic assays, as well. Such an application takes advantage of the sensitivity of the assays of the invention to events which take place at characteristic stages in the progression of a tumor. For example, a given marker gene may be up or downregulated at a very early stage, perhaps before the cell is irreversibly committed to developing into a malignancy, while another marker gene may be characteristically up or down regulated only at a much later stage. Such a method could involve the steps of contacting the mRNA of a test cell with a nucleic acid probe derived from a given marker nucleic acid which is expressed at different characteristic levels in cancerous or precancerous cells at different stages of tumor progression, and determining the approximate amount of hybridization of the probe to the mRNA of the cell, such amount being an indication of the level of expression of the gene in the cell, and thus an indication of the stage of tumor progression of the cell; alternatively, the assay can be carried out with an antibody specific for the gene product of the given marker nucleic acid, contacted with the proteins of the test cell. A battery of such tests will disclose not only the existence and location of a tumor, but also will allow the clinician to select the mode of treatment most appropriate for the tumor, and to predict the likelihood of success of that treatment.

The methods of the invention can also be used to follow the clinical course of a tumor. For example, the assay of the invention can be applied to a tissue sample from a patient; following treatment of the patient for the cancer, another tissue sample is taken and the test repeated. Successful treatment will result in either removal of all cells which demonstrate differential expression characteristic of the cancerous or precancerous cells, or a substantial increase in expression of the gene in those cells, perhaps approaching or even surpassing normal levels.

In yet another embodiment, the invention provides methods for determining whether a subject is at risk for developing a disease, such as a predisposition to develop cancer,.for example colon cancer, associated with an aberrant activity of any one of the polypeptides encoded by nucleic acids of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, wherein the aberrant activity of the polypeptide is characterized by detecting the presence or absence of a genetic lesion characterized by at least one of (i) an: alteration affecting the integrity of a gene encoding a marker polypeptides, or (ii) the mis-expression of the encoding nucleic acid. To illustrate, such genetic lesions can be detected by ascertaining the existence of at least one of(i) a deletion of one or more nucleotides from the nucleic acid sequence, (ii) an addition of one or more nucleotides to the nucleic acid sequence, (iii) a substitution of one or more nucleotides of the nucleic acid sequence, (iv) a gross chromosomal rearrangement of the nucleic acid sequence,. (v) a gross alteration in the level of a messenger RNA transcript of the nucleic acid sequence, (vii) aberrant modification of the nucleic acid sequence, such as of the methylation pattern of the genomic DNA, (vii) the presence of a non-wild type splicing pattern of a messenger RNA transcript of the gene, (viii) a non-wild type level of the marker polypeptide, (ix) allelic loss of the gene, and/or (x) inappropriate post-translational modification of the marker polypeptide.

The present invention provides assay techniques for detecting lesions in the encoding nucleic acid sequence. These methods include, but are not limited to, methods involving sequence analysis, Southern blot hybridization, restriction enzyme site mapping, and methods involving detection of absence of nucleotide pairing between the nucleic acid to be analyzed and a probe.

Specific diseases or disorders, e.g., genetic diseases or disorders, are associated with specific allelic variants of polymorphic regions of certain genes, which do not necessarily encode a mutated protein. Thus, the presence of a specific allelic variant of a polymorphic region of a gene in a subject can render the subject susceptible to developing a specific disease or disorder. Polymorphic regions in genes, can be identified, by determining the nucleotide sequence of genes in populations of individuals. If a polymorphic region is identified, then the link with a specific disease can-be determined by studying specific populations of individuals, e.g, individuals which developed a specific disease, such as colon cancer. A polymorphic region can be located in any region of a gene, e.g., exons, in coding or non coding regions of exons, introns, and promoter region.

In an exemplary embodiment, there is provided a nucleic acid composition comprising a nucleic acid probe including a region of nucleotide sequence which is capable of hybridizing to a sense or antisense sequence of a gene or naturally occurring mutants thereof, or 5′ or 3′ flanking sequences or intronic sequences naturally associated with the subject genes or naturally occurring mutants thereof. The nucleic acid of a cell is rendered accessible for hybridization, the probe is contacted with the nucleic acid of the sample, and the hybridization of the probe to the sample nucleic acid is detected. Such techniques can be used to detect lesions or allelic variants at either the genomic or mRNA level, including deletions, substitutions, etc., as well as to determine mRNA transcript levels.

A preferred detection method is allele specific hybridization using probes overlapping the mutation or polymorphic site and having about 5, 10, 20, 25, or 30 nucleotides around the mutation or polymorphic region. In a preferred embodiment of the invention several probes capable of hybridizing specifically to allelic variants are attached to a solid phase support, e.g., a “chip”. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

In certain embodiments, detection of the lesion comprises utilizing the probe/primer in a polymerase chain reaction (PCR) (see, e.g. U.S. Pat. Nos. 4,683,195 and 4,683,202), such as anchor PCR or RACE PCR, or, alternatively, in a ligase chain reaction. (LCR) (see, e.g., Landegren et al. (1988) Science 241:1077-1080; and Nakazawa et al. (1994) PNAS 91:360-364), the latter of which can be particularly usefull for detecting point mutations in the gene (sec Abravaya et al. (1995) Nuc Acid Res 23:675-682). In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize to a nucleic acid sequence under conditions such that hybridization and amplification of the nucleic acid (if present) occurs, and (iv) detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., 1990, Proc. Natl., Acad. Sci. USA 87:1874-1878), transcriptional amplification system (Kwoh, D. Y. et al., 1989, Proc. NatI. Acad. Sci. USA 86:1173-11₇₇), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In a preferred embodiment of the subject assay, mutations in, or allelic variants, of a gene from a sample cell are identified by alterations in restriction enzyme cleavage patterns. For example, sample and control DNA is isolated, amplified (optionally), digested with one or more restriction endonucleases, and fragment length sizes are determined by gel electrophoresis. Moreover, the use of sequence specific ribozymes (see, for example, U.S. Pat. No. 5,498,531) can be used to score for the presence of specific mutations by development or loss of a ribozyme cleavage site.

Another aspect of the invention is directed to the identification of agents capable of modulating the differentiation and proliferation of cells characterized by aberrant proliferation. In this regard, the invention provides assays for determining compounds that modulate the expression of the marker nucleic acids (SEQ ID NO: 27; SEQ ID NO: 29; SEQ ID NO: :31; SEQ ID NO: 33; SEQ ID NO: 35; SEQ ID NO: 37; SEQ ID NO: 38; SEQ ID NO: 40; SEQ:ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61) and/or alter for example, inhibit the bioactivity of the encoded-polypeptide.

Several in vivo methods can be used to identify compounds that modulate expression of the marker nucleic acids (SEQ ID NO: 27; SEQ ID NO: 29; SEQ ID NO: 31; SEQ ID NO: 33; SEQ ID NO: 35; SEQ ID NO: 37, SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO 61) and/or-alter for example, inhibit the bioactivity of the encoded polypeptide.

Drug screening is performed by adding a test compound to a sample of cells, and monitoring the effect. A parallel sample which does not receive the test compound is also monitored as a control. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, and the ability of the cells to interact with other cells or compounds. Differences between treated and untreated cells indicates effects attributable to the test compound.

Desirable effects of a test compound include an effect on any phenotype that was conferred by the cancer-associated marker nucleic acid sequence. Examples include a test compound that limits the overabundance of mRNA, limits production of the encoded protein, or limits the functional effect of the protein. The effect of the test compound would be apparent when comparing results between treated and untreated cells

The invention thus also encompasses methods of screening for agents which inhibit expression of the nucleic acid markers (SEQ ID NO: 27; SEQ ID NO: 29; SEQ ID NO: 31; SEQ ID NO:33; SEQ ID NO: 35; SEQ ID NO: 37; SEQ ID NO: 38; SEQ ID NO:40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61) in vitro, comprising exposing a cell or tissue in which the marker nucleic acid mRNA is detectable in cultured cells to an agent in order to determine whether, the agent is capable of inhibiting production of the mRNA; and determining the level of mRNA in the exposed cells or tissue, wherein a decrease in the level of the mRNA after exposure of the cell line to the agent is indicative of inhibition of the marker nucleic acid mRNA production.

Alternatively, the screening method may include in vitro screening of a cell or tissue in which marker protein is detectable in cultured cells to an agent suspected of inhibiting production of the marker protein; and determining the level of the marker protein in the cells or tissue, wherein a decrease in the level of marker protein after exposure of the cells or tissue to the agent is indicative of inhibition of marker protein production.

The invention also encompasses in vivo methods of screening for agents which inhibit expression of the marker nucleic acids, comprising exposing a mammal having tumor cells in which marker mRNA or protein is detectable to an agent suspected of inhibiting production of marker mRNA or protein; and determining the level of marker mRNA or protein in tumor cells of the exposed mammal. A decrease in the level of marker mRNA or protein after exposure of the mammal to the agent is indicative of inhibition of marker nucleic acid expression.

Accordingly, the invention provides a method comprising incubating a cell expressing the marker nucleic acids (SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61) with a test compound and measuring the mRNA or protein level. The invention further provides a method for quantitatively determining the level of expression of the marker nucleic acids in a cell population, and a method for determining whether an agent is capable of increasing or decreasing the level of expression of the marker nucleic acids in a cell population. The method for determining whether an agent is capable of increasing or decreasing the level of expression of the marker nucleic acids in a cell population comprises the steps of(a) preparing cell extracts from control and agent-treated cell populations, (b) isolating the marker polypeptides from the cell extracts, (c) quantifying (e.g., in parallel) the amount of an immunocomplex formed between the marker polypeptide and an antibody specific to said polypeptide. The marker polypeptides of this invention may also be quantified by assaying for its bioactivity. Agents that induce increased the marker nucleic acid expression may be identified by their ability to increase the amount of immunocomplex formed in the treated cell as compared with the amount of the immunocomplex formed in the control cell. In- a similar manner, agents that decrease expression of the marker nucleic acid may be identified by their ability to decrease the amount of the immunocomplex formed in the treated cell extract as compared to the control cell.

mRNA levels can be determined by Northern blot hybridization. mRNA levels can also be determined by methods involving PCR. Other sensitive methods for measuring mRNA, which can be used in high throughput assays, e.g.; a method using a DELFIA endpoint detection and quantification method, are described, e.g., in Webb and Hurskainen (1996) Journal of Biomolecular Screening 1:119. Marker protein levels can be determined by immunoprecipitations or immunohistochemistry using an antibody that specifically recognizes the protein product encoded by SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60;.SEQ ID NO: 61.

Agents that are identified as active in the drug screening assay are candidates to be tested for their capacity to block cell proliferation activity. These agents would be useful for treating a disorder involving aberrant growth of cells, especially colon cells.

A variety of assay formats will suffice and, in light of the present disclosure, those not expressly described herein will nevertheless be comprehended by one of ordinary skill in the art.

For instance, the assay can be generated in many different formats, and include assays based on cell-free systems, e.g., purified proteins or cell lysates, as well as cell-based assays which utilize intact cells.

In many drug screening programs which test libraries of compounds and natural extracts, 5′ high throughput assays are desirable in order to maximize the number of compounds surveyed in a given period of time. Assays of the present invention which are performed in cell-free systems, such as may be derived with purified or semi-purified proteins or with lysates, are often preferred as “primary” screens in that they can be generated to permit rapid-development and relatively easy detection of an alteration-in a molecular target which is mediated by a test compound. Moreover, the effects of cellular toxicity, and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

Use of Nucleic Acids, as Probes in, Tissue Profiling Probes

Polynucleotide probes as described above, e g, comprising at least 12 contiguous nucleotides selected from the nucleotide SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID: NO: 61, or a sequence complementary thereto, are used for a variety of purposes, including determining transcription levels.

Nucleotide probes are used to detect expression of a gene corresponding to the nucleic acid. For example, in Northern blots, mRNA is separated electrophoretically and contacted with a probe. A probe is detected as hybridizing to an mRNA species of a particular size. The amount of hybridization is quantitated to determine relative amounts of expression, for example under a particular condition. Probes are also used to detect products of amplification by polymerase chain reaction. The products of the reaction are hybridized to the probe and hybrids are detected. Probes are used for in situ hybridization to cells to detect expression. Probes can also be used in vivo for diagnostic detection of hybridizing sequences. Probes are typically labeled with a radioactive isotope. Other types of detectable labels may be used such as chromophores, fluorophores, and enzymes.

Expression of specific mRNA can vary in different cell types and can be tissue specific. This variation of mRNA levels in different cell types can be exploited with nucleic acid probe assays to determine tissue types. For example, PCR, branched DNA probe assays, or blotting techniques utilizing nucleic acid probes substantially identical or complementary to nucleic acids of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, can determine the presence or absence of target cDNA or mRNA.

Examples of a nucleotide hybridization assay are described in Urdea et al., PCT W092/02-526 and Urdea et al., U.S. Pat. No. 5,124,246, both incorporated herein by reference. The references describe an example of a sandwich nucleotide hybridization assay.

Alternatively, the PCR is another means for detecting small amounts of target nucleic acids, as described in Mullis et al., Met/i. Enzymol. (1987) 155:335-350; U.S. Pat. No. 4,683,195; and U.S. Pat. No. 4,683,202, all incorporated herein by reference. Two primer polynucleotides nucleotides hybridize with the target nucleic acids and are used to prime the reaction. The primers may be composed of sequence within or 3′ and 5′ to the polynucleotides of the Sequence Listing. Alternatively, if the primers are 3′ and 5′ to these polynucleotides, they need not hybridize to them or the complements. A thermostable polymerase creates copies of target nucleic acids from the primers using the original target nucleic acids as a template. After a large amount of target nucleic acids is generated by the polymerase, it is detected by methods such as Southern blots. When using the Southern blot method, the labeled probe will hybridize to a polynucleotide of the Sequence Listing or complement.

Furthermore, mRNA or cDNA can be detected by traditional blotting techniques described in Sambrook et al., “Molecular Cloning: A Laboratory Manual” (New York; Cold Spring Harbor Laboratory, 1989). mRNA or cDNA generated from mRNA using a polymerase enzyme can be purified and separated using gel electrophoresis. The nucleic acids on the gel are then blotted onto a solid support, such as nitrocellulose. The solid support is exposed to a labeled probe and then washed to remove any unhybridized probe. Next, the duplexes containing the labeled probe are detected. Typically, the probe is labeled with radioactivity.

Tissue Profiling

The nucleic acids of the present invention can be used to determine the tissue type from which a given sample is derived. For example, a metastatic lesion is identified by its developmental organ or tissue source by identifying the expression of a particular marker of that organ or tissue. If a nucleic acid is expressed only in a specific tissue type, and a metastatic lesion is found to express that nucleic acid, then the developmental source of the lesion has been identified. Expression of a particular nucleic acid is assayed by detection of either the corresponding mRNA or the protein product. Immunological methods, such as antibody staining, are used to detect a particular protein product. Hybridization methods may be used to detect particular mRNA species, including but not limited to in situ hybridization and Northern blotting.

Use of Nucleic Acids and Encoded Polypeptides to Raise Antibodies

Expression products of a nucleic acid, the corresponding mRNA or cDNA, or the corresponding complete gene are prepared and used for raising antibodies for experimental, diagnostic, and therapeutic purposes. For nucleic acids to which a corresponding gene has not been assigned, this provides an additional method of identifying the corresponding gene. The nucleic, acid or related cDNA is expressed as described above, and antibodies are prepared. These antibodies are specific to an epitope on the encoded polypeptide, and can precipitate or bind to the corresponding native protein in a cell or tissue preparation or in a cell-free extract of an in vitro expression system.

Immunogens for raising antibodies are prepared by mixing the polypeptides encoded by the nucleic acids of the present invention with adjuvants. Alternatively, polypeptides are made as fusion proteins to larger immunogenic proteins.. Polypeptides are also covalently linked to other larger immunogenic proteins, such as keyhole limpet hemocyanin. Immunogens are typically administered intradermally, subcutaneously, or intramuscularly. Immunogens are administered to experimental animals such as rabbits, sheep, and mice, to generate antibodies. Optionally, the animal spleen cells are isolated and fused with myeloma cells to form hybridomas which secrete monoclonal antibodies. Such methods are well known in the art. According to another method known in the art, the nucleic acid is administered directly, such as by intramuscular injection, and expressed in vivo. The expressed protein generates a variety of protein-specific immune responses, including production of antibodies, comparable to administration of the protein.

Preparations of polyclonal and monoclonal antibodies specific for nucleic acid-encoded proteins and polypeptides are made using standard methods known in the art. The antibodies specifically bind to epitopes present in the polypeptides encoded by a nucleic acid of SEQ ID NO: 27; SEQ ID NO: 29; SEQ ID NO: 31; SEQ ID NO: 33; SEQ ID NO: 35; SEQ ID) NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto. In another embodiment, the antibodies specifically bind to epitopes present in a polypeptide encoded by SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50;SEQ ID NO: 51; SEQ ID NO: 52; SEQ ID NO: 60; SEQ ID NO: 61. Typically, at least about 6, 8, 10, or 12 contiguous amino acids are required to form an epitope. However, epitopes which involve noncontiguous amino acids may require more, for example, at least about 15, 25, or 50 amino acids. A short sequence of a nucleic acid may then be unsuitable for use as an epitope to raise antibodies for identifying the corresponding novel protein, because of the potential for cross-reactivity with a known protein. However, the antibodies may be useful for other purposes, particularly if they identify common structural features of a known protein and a novel polypeptide encoded by a nucleic acid of the invention.

Antibodies that specifically bind to human nucleic acid-encoded polypeptides should provide a detection signal at least about 5-, 10-, or 20-fold higher than a detection signal provided with other proteins when used in Western blots or other immunochemical assays. Preferably, antibodies that specifically bind nucleic acid T-encoded polypeptides do not detect other proteins in immunochemical assays and can immunoprecipitate nucleic acid-encoded proteins from solution.

To test for the presence of serum antibodies to the nucleic acid-encoded polypeptide in a human population, human antibodies are purified by methods well known in the art. Preferably, the antibodies ate affinity purified by passing antiserum over a column to which a nucleic acid-encoded protein, polypeptide, or fusion protein is bound. The bound antibodies can then be eluted from the column, for example using a buffer with a high salt concentration.

In addition to the antibodies discussed above, genetically engineered antibody derivatives are made, such as single chain antibodies.

Antibodies may be made by using standard protocols known in the art (See, for example, Antibodies: A Laboratory Manual ed. by Harlow and Lane (Cold Spring Harbor Press. 1988)). A mammal, such as a mouse, hamster, or rabbit can be immunized with an immunogenic form of the peptide (e.g., a mammalian polypeptide or an antigenic fragment which is capable of eliciting an antibody response, or a fusion protein as described above).

In one aspect, this invention includes monoclonal antibodies that show a subject polypeptide is highly expressed in colorectal tissue or tumor tissue, especially colon cancer tissue or colon cancer-derived cell lines. Therefore, in one embodiment, this invention provides a diagnostic tool for the analysis of expression of a subject polypeptide in general, and in particular, as a diagnostic for colin cancer.

Techniques for conferring immunogenicity on a protein or peptide include conjugation to carriers or other techniques well-known in the art. An immunogenic portion of a protein can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of a protein of a mammal, e.g., antigenic determinants of a protein encoded by one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 or closely related homologs (e.g., at least 90% identical, and more preferably at least 95% identical).

Following immunization of an animal with an antigenic preparation of a polypeptide, antisera can be obtained and, if desired, polyclonal antibodies isolated from the serum. To produce monoclonal antibodies, antibody-producing cells (lymphocytes) can-be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a polypeptide of the present invention and monoclonal antibodies isolated from a culture comprising- such hybridoma cells.

The term antibody as used herein is intended to include fragments thereof which are also specifically reactive with one of the subject polypeptides. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to, produce Fab fragments. The antibody of the present invention is further intended to include bispecific, single-chain, and chimeric and humanized molecules having affinity for a polyep tide conferred by at least one CDR region of the antibody. In preferred embodiments, the antibodies, the antibody further comprises a label attached thereto and able to be detected, (e.g., the label can be a radioisotope, fluorescent compound, chemiluminescent compound, enzyme, or enzyme co-factor).

Antibodies can be used, e.g., to monitor protein levels in an individual for determingin, e.g., whether a subject has a disease or condition, such as colon cancer, associated with an aberrant protein level, or allowing determination of the efficacy of a given treatment regimen for an individual afflicted with such a disorder. The level of polypeptides may be measured from cells in bodily fluid, such as in blood samples.

Another application of antibodies of the present invention is in the immunological screening of cDNA libraries constructed in expression vectors such as gtl1, gtl8-23, ZAP, and ORF8. Messenger libraries of this type, having coding sequences inserted in the correct reading frame and orientation, can produce fusion proteins. For instance, gtl 1 will produce fusion proteins whose amino termini consist of β-galactosidase amino acid sequences and whose carboxyl termini consist of a foreign polypeptide. Antigenic epitopes of a protein, e.g., other orthologs of a particular protein or other paralogs from the same species, can then be detected with antibodies, as, for example, reacting nitrocellulose filters lifted from infected plates with antibodies. Positive phage detected by this assay can then be isolated from the infected plate. Thus, the presence of homologs can be detected and cloned from other animals, as can alternate isoforms (including splicing variants) from humans.

In another embodiment, a panel of monoclonal antibodies may be used, wherein each of the epitope's involved functions are represented-by a monoclonal antibody. Loss or perturbation of binding of a monoclonal antibody in the panel would be indicative of a mutational attention of the protein and thus of the corresponding gene.

Differential Expression

The present invention also provides a method to identify abnormal or diseased tissue in a human. For nucleic acids corresponding to profiles of protein families as described above, the choice of tissue may be dictated by the putative biological function. The expression of a gene corresponding to a specific nucleic acid is compared between a first tissue that is suspected of being diseased and a second, normal tissue of the human. The normal tissue is any tissue of the human, especially those that express the target gene including, but not limited to, brain, thymus, testis, heart, prostate, placenta, spleen, small intestine, skeletal muscle, pancreas, and the mucosal lining of the colon.

The tissue suspected of being abnormal or diseased can be derived from a different tissue type of the human, but preferably it is derived from the same tissue type; for example an intestinal polyp or other abnormal growth should be compared with normal intestinal tissue. A difference between the target gene, mRNA, or protein in the two tissues which are compared, for example in molecular weight, amino acid or nucleotide sequence, or relative abundance, indicates a change in the gene, or a gene which regulates it, in the tissue of the human that was suspected of being diseased.

The target genes in the two tissues are compared by any means known in the art. For example, the two genes are sequenced, and the sequence of the gene in the tissue suspected of being diseased is compared with the gene sequence in the normal tissue. The target genes, or portions thereof, in the two tissues are amplified, for example using nucleotide primers based on the nucleotide sequence shown in the Sequence Listing, using the polymerase chain reaction. The amplified genes or portions of genes are hybridized to nucleotide probes selected from a corresponding-nucleotide sequence shown SEQ ID NO: 27; SEQ ID NO: 29; SEQ ID NO: 31; SEQ ID NO:33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61. A difference in the nucleotide sequence of the target gene in the tissue suspected of being diseased compared with the normal nucleotide sequence suggests a role of the nucleic acid-encoded proteins in the disease and provides a lead for preparing a therapeutic agent. The nucleotide probes are labeled by a variety of methods, such as radiolabeling, biotinylation, or labeling with fluorescent or chemiluminescent tags, and detected by standard methods known in the art.

Alternatively, target mRNA in the two,tissues is compared. PolyA⁺RNA is isolated from the two tissues as is known in the art. For example, one of skill in the art can readily determine differences in the size or amount of target mRNA transcripts between the two tissues using Northern blots and nucleotide probes selected from the nucleotide sequence shown in the Sequence Listing. Increased or decreased expression of a target mRNA in a tissue sample suspected of being diseased, compared with the expression of the same target mRNA in a normal tissue, suggests that the expressed protein has a role in the disease, and also provides a lead for preparing a therapeutic agent.

Any method for analyzing proteins is used to compare two nucleic acid-encoded proteins from matched samples. The sizes of the proteins in the two tissues are compared, for example, using antibodies of the present invention to detect nucleic acid-encoded proteins in Western blots of protein extracts from the two tissues. Other changes, such as expression levels and subcellular localization, can also be detected immunologically, using antibodies to the corresponding protein. A higher or lower level of nucleic acid-encoded protein expression in a tissue suspected of being diseased, compared with the same nucleic acid-encoded protein expression level in a normal tissue, is indicative that the expressed protein has a role in the disease, and provides another lead for preparing a therapeutic agent.

Similarly, comparison of gene sequences or of gene expression products, e.g., mRNA and protein, between a human tissue that is suspected of being diseased and a normal tissue of a human, are used to follow disease progression or remission in the human. Such comparisons of genes, mRNA, or protein are made as described above.

For example, increased or decreased expression of the target gene in the tissue suspected of being neoplastic can indicate the presence of neoplastic cells in the tissue. The degree of increased expression of the target gene in the neoplastic tissue relative to expression of the gene in normal tissue, or differences in the amount of increased expression of the target gene in the neoplastic tissue over time, is used to assess the progression of the neoplasia in that tissue or to monitor the response of the neoplastic tissue to a therapeutic protocol over time.

The expression pattern of any two cell types can be compared, such as low and high metastatic tumor cell lines, or cells from tissue which have and have not been exposed to a therapeutic agent. A genetic predisposition to disease in a human is detected by comparing an target gene, mRNA, or protein in a fetal tissue with a normal target gene, mRNA, or protein. Fetal tissues that are used for this purpose include, but are not limited to, amniotic fluid, chorionic villi, blood, and the blastomere of an in vitro fertilized embryo. The comparable normal target gene is obtained from any tissue. The mRNA or protein is obtained from a normal tissue of a human in which the target gene is expressed. Differences such as alterations in the nucleotide sequence or size of the fetal target gene or mRNA, or alterations in the molecular weight, amino acid sequence, or relative abundance of fetal target protein, can indicate a germline mutation in the target gene of the fetus, which indicates a genetic predisposition to disease.

Use of Nucleic Acids and Encoded Polypeptides to Screen for Peptide Analogs and Antagonists

Polypeptides encoded by the instant nucleic acids, e.g., or a sequence complementary thereto, and corresponding full length genes can be used to screen peptide libraries to identify binding partners, such as receptors, from among the encoded polypeptides.

A library of peptides may be synthesized following the methods disclosed in U.S. Pat. No. 5,010,175, and in PCT WO 91/17823. As described below in brief, one prepares a mixture of peptides, which is then screened to identify the peptides exhibiting the desired signal transduction and receptor binding activity. In the '175 method, a suitable peptide synthesis support (e.g., a resin) is coupled to a mixture of appropriately protected, activated amino acids. The concentration of each amino acid in the reaction mixture is balanced or adjusted in inverse proportion to its coupling reaction rate so that the product is an equimolar mixture of amino acids coupled to the starting resin. The bound amino acids are then deprotected, and reacted with another balanced amino acid mixture to form an equimolar mixture of all possible dipeptides. This process is repeated until a mixture of peptides of the desired length (e.g., hexamers) is formed. Note that one need not include all amino acids in each step: one may include only one or two amino acids in some steps (e.g., where it is known that a particular amino acid is essential in a given position), thus reducing the complexity of the mixture. After the synthesis of the peptide library is completed, the mixture of peptides is screened for binding to the selected polypeptide. The peptides are then tested for their ability to inhibit or enhance activity. Peptides exhibiting the desired activity are then isolated and sequenced.

The method described in WO 91/17823 is similar. However, instead of reacting the synthesis resin with a mixture of activated amino acids, the resin is divided into twenty equal portions (or into a number of portions corresponding to the number of different amino acids to be added in that step), and each amino acid is coupled individually to its portion of resin. The resin portions are then combined, mixed, and again divided into a number of equal portions for reaction with the second amino acid. In this manner, each reaction may be easily driven to completion. Additionally, one may maintain separate “subpools” by treating portions in parallel, rather than combining all resins at each step. This simplifies the process of determining which peptides are responsible for any observed receptor binding or signal transduction activity.

In such cases, the subpools containing, e.g., 1-2,000 candidates each are exposed to one or more polypeptides of the invention. Each subpool that produces a positive result is then resynthesized as a group of smaller subpools (sub-subpools) containing, e.g., 20-100 candidates, and reassayed. Positive sub-subpools may be resynthesized as individual compounds, and assayed finally to determine the peptides that exhibit a high binding constant. These peptides can be tested for their ability to inhibit or enhance the native activity. The methods described in WO 91/7823 and U.S. Pat. No. 5,194,392 (herein incorporated by reference) enable the preparation of such pools and subpools by automated techniques in parallel, such that all synthesis and resynthesis may be performed in a matter of days.

Peptide agonists or antagonists are screened using any available method, such as signal transduction, antibody binding, receptor binding, mitogenic assays, chemotaxis assays, etc. The methods described herein are presently preferred. The assay conditions ideally should resemble the conditions under which the native activity is exhibited in vivo, that is under physiologic pH, temperature, and ionic strength. Suitable-agonists or antagonists will exhibit strong inhibition or enhancement of the native activity at concentrations that do not cause toxic side effects in the subject. Agonists or antagonists that compete for binding to the native polypeptide may require concentrations equal to or greater than the native concentration, while inhibitors capable of binding irreversibly to the polypeptide may be added in concentrations on the order of the native concentration.

The end results of such screening and experimentation will be at least one novel polypeptide binding partner, such as a receptor, encoded by a nucleic acid of the invention, and at least one peptide agonist or antagonist of the novel binding partner. Such agonists and antagonists can be used to modulate, enhance, or inhibit receptor function in cells to which the receptor is native, or in cells that possess the receptor as a result of genetic engineering. Further, if the novel receptor shares biologically important characteristics with a known receptor, information about agonist/antagonist binding may help in developing improved agonists/antagonists of the known receptor.

Pharmaceutical Compositions and Therapeutic Uses

Pharmaceutical compositions can comprise polypeptides, antibodies, or polynucleotides of the claimed invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased-body temperature. The precise effective amount for a subject will depend upon the subject's size and health, the nature and extent of the condition, and the therapeutics or combination of therapeutics selected for administration. Thus, it is not, useful, to specify an exact effective amount in advance. However, the effective amount for a given situation can be determined by routine experimentation and is within the judgment of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances; and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Delivery Methods

Once formulated, the nucleic acids compositions of the invention can be (I) administered directly to the subject; (2) delivered ex vivo, to cells derived from the subject or (3) delivered in vitro for expression of recombinant proteins.

Direct delivery of the compositions will generally be accomplished by injection, either subcutaneously, intraperitoneally, intravenously or intramuscularly, or delivered to the interstitial space of a tissue. The compositions can also be administered into a tumor or lesion. Other modes of administration include oral and pulmonary administration, suppositories, and transdermalt applications, needles, and gene guns or hyposprays. Dosage treatment may be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Once a subject gene has been found to correlate with a proliferative disorder, such as neoplasia, dysplasia, and hyperplasia, the disorder may be amenable to treatment by administration of a therapeutic agent based on the nucleic acid or corresponding polypeptide.

Preparation of antisense polypeptides is discussed above. Neoplasias that are treated with the antisense composition include, but are not limited to, cervical cancers, melanomas, colorectal adenocarcinomas, Wilms' tumor, retinoblastoma, sarcomas, ryosarcomas; lung carcinomas, leukemias, such, as chronic myelogenous leukemia, promyelocytic leukemia, monocytic leukemia, and myeloid leukemia, and lymphomas, such as histiocytic lymphoma. Proliferative disorders that are treated with the therapeutic composition include disorders such as anhydric hereditary ectodermal dysplasia, congenital alveolar dysplasia, epithelial dysplasia of the cervix, fibrous dysplasia of bone, and mammary dysplasia. Hyperplasias, for example, endometrial, adrenal, breast, prostate, or thyroid hyperplasias or pseudoepitheliomatous hyperplasia of the skin, are treated with antisense therapeutic compositions. Even in disorders in which mutations in the corresponding gene are not implicated, downregulation or inhibition of nucleic acid-related gene expression can have therapeutic application. For example, decreasing nucleic acid-related gene expression can help to suppress tumors in which enhanced expression of the gene is implicated.

Both the dose of the antisense composition and the means of administration are determined based on the specific qualities of the therapeutic composition, the condition, age, and weight of the patient, the progression of the disease, and other relevant factors. Administration of the therapeutic antisense agents of the invention includes local or systemic administration, including injection, oral administration, particle gun or catheterized administration, and topical administration. Preferably, the therapeutic antisense composition contains an expression construct comprising a promoter and a polynucleotide segment of at least about 12, 22, 25, 30, or 35 contiguous nucleotides of the antisense strand of a nucleic acid. Within the expression construct, the polynucleotide segment is located downstream from the promoter, and transcription of the polynucleotide segment initiates at the promoter.

Various methods are used to administer the therapeutic composition directly to a specific site in the body. For example, a small metastatic lesion is located and the therapeutic composition injected several times in several different locations within the body of tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. The antisense composition is directly administered to the surface of the tumor, for example, by topical application of the composition. X-ray imaging is used to assist in certain of the above delivery methods.

Receptor-mediated targeted delivery of therapeutic compositions containing an antisense polynucleotide, subgenomic polynucleotides, or antibodies to specific tissues is also used. Receptor-mediated DNA delivery techniques are described in, for example, Findeis et al. Trends in Biotechnol. (1993) 11:202-205; Chiou et al., (1994) Gene Therapeutics: Methods And Applications Of Direct Gene Transfer (J. A. Wolff, ed.); Wu & Wu, J. Biol. Chem. (1988) 263:621-24; Wu et al., J. Biol. Chem. (1994) 269:542-46; Zenke et al., Proc. Nail. Acad. Sci. (USA) (1990) 87:3655-59; Wu et al., J. Biol. Chem. (1991) 266.33.8-42. Preferably, receptor-mediated targeted delivery of therapeutic compositions containing antibodies of the invention is used to deliver the antibodies to specific tissue.

Therapeutic compositions containing antisense subgenomic polynucleotides are administered in a range of about 100 ng to about 200 mg of DNA for local administration in a gene therapy protocol. Concentration ranges of about 500 ng to about 50 mg, about I mg to about 2 mg, about 5 mg to about 500 mg, and about 20 mg to about 100 mg of DNA can also be used during a gene therapy protocol. Factors such as method of action and efficacy of transformation and expression are considerations which will affect the dosage required for ultimate efficacy of the antisense subgenomic nucleic acids. Where greater expression is desired over a larger area of tissue, larger amounts of antisense subgenomic nucleic acids or the same amounts readministered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect. A more complete description of gene therapy vectors, especially retroviral vectors, is contained in U.S. Ser. No. 08/869,309, which is expressly incorporated herein, and in section F below.

For genes encoding polypeptides or proteins with anti-inflammatory activity, suitable use, doses, and administration are described in U.S. Pat. No. 5,654,173, incorporated herein by reference. Therapeutic agents also include antibodies to proteins and polypeptides encoded by the subject nucleic acids, as described in U.S. Pat. No. 5,654,173.

Transgenic Animals

One aspect of the present invention relates to transgenic non-human animals having germline and/or somatic cells in which the biological activity of one or more genes are altered by a chromosomally incorporated transgene.

In a preferred embodiments, the transgene encodes a mutant protein, such as dominant negative protein which antagonizes at least a portion of the biological function of a wild-type protein.

Yet another preferred transgenic animal includes a transgene encoding an antisense transcript which, when transcribed from the transgene, hybridizes with a gene or a mRNA transcript thereof, and inhibits expression of the gene.

In one embodiment, the present invention provides a desired non-human animal or an animal (including human) cell which contains a predefined, specific and desired alteration rendering the non-human animal or animal cell predisposed to cancer. Specifically, the invention pertains to a genetically altered non-human animal (most preferably, a mouse), or a cell (either non-human animal or human) in culture, that is defective in at least one of two alleles of a tumor-suppressor gene. The inactivation of at least one of these tumor suppressor alleles results in an animal with a higher susceptibility to tumor induction or other proliferative or differentiative disorders, or disorders marked by aberrant signal transduction, e.g., from a cytokine or growth factor. A genetically altered mouse of this type is able to serve as a useful model for hereditary cancers and as a test animal for carcinogen studies. The invention additionally pertains to the use of such non-human animals or animal cells, and their progeny in research and medicine.

Furthermore, it is contemplated that cells of the transgenic animals of the present invention can include other transgenes, e.g., which alter the biological activity of a second tumor suppressor gene or an oncogene. For instance, the second transgene can functionally disrupt the biological activity of a second tumor suppressor gene, such as p53, p73, DCC, p21^{ciP1}, p27^{kp1}, Rb, Mad or E2F. Alternatively, the second transgene can cause overexpression or loss of regulation of an oncogene, such as ras, myc, a cdc25 phosphatase, Bcl-2, Bcl-6, a transforming growth factor,-neu, int-3, polyoma virus middle T antigen, SV40 large T antigen, a papillomaviral E6 protein, a papillomaviral E7 protein, CDK4, or cyclin D1.

A preferred transgenic non-human animal of the present invention has germline and/or somatic cells in which one or more alleles of a gene are disrupted by a chromosomally incorporated transgene, wherein the transgene includes a marker sequence providing a detectable signal for identifying the presence of the transgene in cells of the transgenic animal, and replaces at least a portion of the gene or is inserted into the gene or disrupts expression of a wild-type protein.

Still another aspect of the present invention relates to methods for generating non-human animals and stem cells having a functionally disrupted endogenous gene. In a preferred embodiment, the method comprises the steps of:
(i) constructing a transgene construct including (a) a recombination region having at least a portion of the gene, which recombination region directs recombination of the transgene with the gene, and (b) a marker sequence which provides a detectable signal for identifying the presence of the transgene in a cell;
(ii) transferring the transgene into stern cells of a non-human animal;
(iii) selecting stem cells having a correctly targeted homologous recombination between the transgene and the gene;
(iv) transferring cells identified in step (iii) into a non-human blastocyst and implanting the resulting chimeric blastocyst into a non-human female; and
(v) collecting offspring harboring an endogenous gene allele having the correctly targeted recombination.

Yet another aspect of the invention provides a method for evaluating the carcinogenic potential of an agent by (i) contacting a transgenic animal of the present invention with a test agent, and (ii) comparing the number of transformed cells in a sample from the treated animal with the number of transformed cells in a sample from an untreated transgenic animal or transgenic animal treated with a control agent. The difference in the number of transformed cells in the treated animal, relative to the number of transformed cells in the absence of treatment-with a control agent, indicates the carcinogenic potential of the test compound.

Another aspect of the invention provides a method of evaluating an anti-proliferative activity of a test compound. In preferred embodiments, the method includes contacting a transgenic animal of the present invention, or a sample of cells from such animal, with a test agent, and determining the number of transformed cells in a specimen from the transgenic animal or in the sample of cells. A statistically significant decrease in the number of transformed cells, relative to the number of transformed cells in the absence of the test agent, indicates the test compound is a potential anti-proliferative agent.

The practice of the present invention will employ, unless otherwise indicated, conventional techniques of cell-biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (ERL Press, 1986); B. Perbal, A Practical Guide To Molecular Cloning (1984); the treatise, Methods in Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

As mentioned above, the sequences described herein are believed to have particular utility in regards to colon cancer. However, they may also be useful with other types of cancers and other disease states.

The present invention will now be illustrated by reference to the following examples which set forth particularly advantageous embodiments. However, it should-be noted that these embodiments are illustrative and are not to be construed as restricting the invention in anyway.

### EXAMPLES

Two different techniques were employed to clone the present novel sequences; (1) a yeast two-hybrid system, and (2) low stringency PCR amplification.

The approaches used to-identify the present sequences provide substantive information regarding their specific tissue of origin, the nature of their biologic interactions, and their likely importance in cellular growth regulation. The technology used to screen for the present newly discovered sequences is unique from the standpoint of (1) utilizing unique cDNA libraries, and (2) utilizing methods that differ from other methods previously used to isolate gene sequences (e.g., ESTs that simply reflect composition of matter) that have partial homology to some of the present sequences.

Identification of Sequences According to the Present Invention using a Yeast-two Hybrid System

The yeast two-hybrid assay used in the present invention was based on the process developed by Fields and coworkers (Nature, 340:245-247,1989). The yeast two-hybrid assay is a yeast-based genetic assay designed to detect protein-protein interactions in vitro. A positive result obtained with the two-hybrid assay allows detection of the presence of genes, for example from a CDNA library, which genes encode candidate proteins that interact with a target protein (“bait”). The method is based on the fact that many eukaryotic transcriptional activator proteins consist of two physically separable domains: one acts as the DNA-binding domain (”BD”) and the other as the transcriptional-activation domain (“AD”). In the yeast two-hybrid system, a reporter yeast strain is used that contains a specific DNA sequence, for example, a GAL4 responsive element, that can interact with a recombinant BD protein that is coupled to the bait. This responsive element is upstream of the reporter genes (for example, Lac Z and HIS3) that are regulated by this element. In addition, this responsive element is linked to a promoter sequence that can interact with a recombinant AD protein that is coupled to a library of candidate proteins. Both BD and AD domains are required for normal activation of transcription in cells or in vitro. Transcription can then be initiated by involving other components of the transcription machinery. In cells, both domains are normally part of the same protein, while in the yeast two-hybrid system, a functional activator protein can be assembled as a protein complex (BD-target protein and an AD-library protein).

In the present method two cDNA libraries were used: one derived from fresh normal colonic epithelium isolated by a previously reported method (Cell. Dev. Biol.-Animal, 33:18-27,1997) and the other from cultured colon cancer HCT 116 cells (ATCC No. CCL-247). In both libraries, the cDNA fragments were cloned into the EcoRI site downstream of the GAL4-AD domain using the fusion vector pGAD10 (CLONTECH Laboratories, Inc., Palo Alto, Calif., according to the manufacturer's instructions).

TABLE 1
Sources and Characteristics of the cDNA Libraries.

| | Priming | | # Independent | Insert Size Range |
|---|---|---|---|---|
| Library Origin | Method | Vector | Clones | (Average) |
| Normal colonic | oligo (dT) + random | pGAD10 | 1.5 × 10⁶ | 0.2-3.5 kb (1.0 kb) |
| epithelium isolated | primer | | | |
| from a female patient. | | | | |
| HCT116 Colon | oligo (dT) + random | pGAD10 | 2.3 × 10⁶ | 0.7-4.0 kb (1.6 kb) |
| Carcinoma Cells, | primer | | | |

To isolate novel genes, the MATCHMAKER™ Two-Hybrid System (CLONTECH Laboratories, Inc., Palo Alto, Calif.) was used to screen the cDNA libraries, as described in the protocol PT 1265-1 obtained from CLONTECH Laboratories, Inc. Two recombinant bait vectors were constructed, each of which code for a target polypeptide: (1) a 3′ fragment of the APC gene consisting of nucleotides 6502-8532 and (2) a fragment of the E12 gene consisting of nucleotides 1528-1962 that encode the bHLH motif that binds to the E12-box promoter region. Each sequence was separately inserted downstream of the GAL4-BD in the CLONTECH fusion vector pGBT9. All experiments in these studies were performed using Saccharomyces cerevisiae yeast cell strain HF7c with the following genetic characteristics: MA-Ta, Ura3-52, his3-200, lys2-801, ade2-101, trpl901, leu2-3, 112, gdl4-542, gal8O-538, LYS2: GAL1-HIS3, URA3:(GAL4J7-mers)3-CYC1-lacZ.

After co-transformation of the cDNA library and the bait into the HF7c yeast cells, the cells were plated on SD-TipLeu-His plates and incubated at 30° C. for 7-10 days or until appearance of visible colonies. Assay for β-galactosidase activity was used to confirm the presence of interacting proteins (i.e. the AD-library protein and the BD-target protein) as follows. After nutrient selection on the SD-Trp-Leu-His plates, the yeast cell colonies were transferred to Whatman filters (#3, Whatman Inc, Clifton, N.J.), made permeable by freezing in liquid nitrogen and incubated with Z buffer for 10 hours at 30° C. as described in the CLONTECH PT 1265-1 protocol. Positive clones were detected by their-blue color and the fusion plasmids containing cDNA candidate genes were then isolated from them.

About two million informants in each of the libraries were screened (see Table 1). Several clones with the appropriate yeast phenotype were identified and sequenced. Clones were analyzed for similarities to one another in order to determine if they arose from a single gene and multiple copies of several candidate sequences were identified using both baits. Using the above APC fragment as bait, we obtained seven novel sequences were obtained (designated CATX-1, CATX-2, CATX-3, CATX-4, CATX-5, CATX-6 and CATX-7). Using the E12 fragment, five novel sequences were obtained (designated CATX-11, CATX-12, CATX-13, CATX-14 and CATX-15). Using the cDNA library screen, several known sequences that interact with the E12 target protein were also obtained (e.g. id1, id2, id3).

Clones that were identified in one library were also analyzed for their presence in the other library using PCR (94° C. for 1 min; 52° C. for 1 min; 72° C. for 2 min; the thermal cycle repeated for 30 times). Several clones were found in both libraries, as shown-in Table 2.

TABLE 2
Clones present in the cDNA libraries-determined by PCR amplification.

| | | | Library 1: | Library 2: |
|---|---|---|---|---|
| | | | Normal | Cultured |
| | | | Colonic | HCT116 |
| CLONE | 5′ Primer | 3′ Primer | Epithelium | Cells |
| CATX-1 | agcacttaatattgtaat | tcaaagccctccagagag | + | + |
| | [SEQ ID NO: 1] | [SEQ ID NO: 2] | | |
| CATX-2 | gggatatagacaggcttg | tactgcactccaccctgg | + | − |
| | [SEQ ID NO: 3] | [SEQ ID NO: 4] | | |
| CATX-3 | gagattttgatctatgtt | tcctgaaatcaggtgatc | + | + |
| | [SEQ ID NO: 5] | [SEQ ID NO: 6] | | |
| CATX-4 | gggaggagccagccctgg | ggtagggttgccaaggtg | + | + |
| | [SEQ ID NO: 7] | [SEQ ID NO: 8] | | |
| CATX-5 | gtgcccaggagtttgaga | ccaatacctacaaattcc | + | + |
| | [SEQ ID NO: 9] | [SEQ ID NO: 10] | | |
| CATX-6 | gggaggagccagccctgg | ggtagggttgccaaggtg | + | NT |
| | [SEQ ID NO: 11] | [SEQ ID NO: 12] | | |
| CATX-7 | gggaggagccagccctgg | ggtagggttgccaaggtg | + | NT |
| | [SEQ ID NO: 13] | [SEQ ID NO: 14] | | |
| CATX-11 | tgctgggattaggactctttgcct | cgtggcagaggaaaagccc | NT | + |
| | ggag | aagttaaag | | |
| | [SEQ ID NO: 15] | [SEQ ID NO: 16] | | |
| CATX-13 | cttacatgtgcttggacgggaat | ctgctcgattgagttaaaagc | NT | + |
| | gcc | gctgctg | | |
| | [SEQ ID NO: 17] | [SEQ ID NO: 18] | | |
| CATX-14 | caaccccatcaaaaagtgggca | ggccggtgatgagcattttttc | NT | + |
| | aagg | g | | |
| | [SEQ ID NO: 19] | [SEQ ID NO: 20] | | |
| CATX-15 | acactggtggaagaggagagg | ccacatgccgcaatgagttca | − | + |
| | gaagccaaac | gaacc | | |
| | [SEQ ID NO: 21] | [SEQ ID NO: 22] | | |

“+” means clone present in library; “−” means clone absent from library; “NT” means not tested.

Products obtained using the APC fragment as bait to screen the normal colon-epithelial library with the yeast two-hybrid system include the following clones: CATX-1 [SEQ ID NO:27-] which has 471 bp and is a partial cDNA sequence. According to the treading frame from pGAD10, a peptide having 121 amino acids can be predicted, having the amino acid sequence of SEQ ID NO:28.

CATX-2 [SEQ ID NO:29]has 983 bp. This sequence appears to be a full cDNA clone because it contains an open reading frame including both a start and stop codons. This disclosure represents the first report of the full length coding sequence of this gene. According to the reading frame from the pGAD10 vector, the coding region is 135 bp, and the expected protein has 45 amino acids, which includes a leucine zipper motif in the N-terminus [SEQ ID NO: 30].

CATX-3 [SEQ ID NO: 31] has 301 bp and is a partial cDNA sequence. According to the reading frame from pGAD10, we predict a peptide having 31 amino acids can be predicted [SEQ ID NO: 32]:

CATX-4 [SEQ ID NO:33] has 158 bp, and is a partial cDNA sequence. According to the reading frame from pGAD10, a peptide having 52 amino acids can be predicted [SEQ ID NO: 34].

CATX-5 [SEQ ID NO: 35] has 290 bp and is a partial cDNA sequence. According to the reading frame from pGAD10, a peptide having 78 amino acids can be predicted [SEQ ID NO: 36].

CATX-6 [SEQ ID NO: 37] has 520 bp and is a partial cDNA sequence.

CATX-7 [SEQ ID NO: 38] has 410 bp and is a partial cDNA sequence.

CATX-12 [SEQ ID NO: 39] was studied for homology to previously reported sequences using GenBank (GBALL). The results showed that CATX-12 corresponds (Pvalue=0) to a previously known gene COX1 Human Cytochrome-c Oxidase polypeptide I (GenBank Accession # X93334).

Using E12 as bait to screen the HCT 116 cDNA library with the yeast two-hybrid system the following clones were obtained:

CATX-11 [SEQ ID NO: 40] has 978 bp. Based on the reading from pGAD10, it is a partial cDNA. According to the reading frame from pGAD-10, a peptide having 324 amino acids can be predicted [SEQ ID NO: 41].

CATX-13 [SEQ ID NO: 42] has 326 bp and is a partial cDNA sequence. According to the reading frame from pGAD10, a peptide having 48 amino acids can be predicted [SEQ ID NO: 43].

CATX-14 [SEQ ID NO: 44] has 418 bp and is a partial cDNA sequence. According to the reading frame from pGAD10, a peptide having 66 amino acids can be predicted [SEQ ID NO: 45].

CATX-15 [SEQ ID NO: 46] has 3032 bp. It appears to be a complete cDNA. This disclosure represents the first report of the full length sequence of this transcript. According to the reading frame from pGAD 10, a peptide having 246 amino acids can be predicted [SEQ ID NO: 47].

Low-Stringency PCR Amplification of Colonic Epithelial Library using Degerate Primers Designed from SLC Sequence

PCR was used to clone sequences having homology with the SCL gene from the normal human colon epithelial cDNA library. The first primer was constructed using a degenerate primer design based on the SCL conservative region consisting of the amino acid sequence ERWR. Accordingly, the primer nucleotide acid sequence, was CCGCCATCGCTC (antisense) [SEQ ID NO:23]. The second primer was a common primer corresponding-to the pGAD10 plasmid region (TACCACTACAATGGATG) [SEQ ID NO:24]. PCR amplification was performed to screen the normal colonic epithelial library using low stringency PCR conditions: 9420 C. for 1 min, 42° C. for 1 min, 72° C. for 3 min, for total of 30 cycles. The PCR products were then separated by agarose gel electrophoresis and purified from the gel, cloned into pGEM®-T vector (Promega, Madison, Wis.) and sequenced. Using the low stringency PCR amplification, three candidate sequences were identified designated CATX-8, CATX-9 and CATX-10.

The following clones-were obtained when PCR was used to screen the normal colonic epithelial cDNA library:

CATX-8 [SEQ ID NO: 48] has 1085 bp and it appears to be a complete cDNA. This represents the first report of the full length sequence of this sequence. Based-on data search, a protein comprising 256 amino acids was predicted [SEQ ID NO: 49].

This sequence contains a motif indicating the presence of ATP and GTP binding domains (AA 15-28 including a p-loop). Analysis for endogenous ATP and GTP binding to CATX-8 was positive (200 ng CATX-8 protein spotted on Nitrocellulose paper and incubated in 20 ml solution containing 50 μCi γ-³²p ATP or GTP in PBS; at 24° C. for 30 min and then washed three times 100 mM sodium phosphate buffer pH 7.2, and then autoradiographed). RT-PCR showed that this protein is expressed in most of human tissues, such as liver, uterus, breast, muscle and colon mucosa. Primer sequences used are given in Table 3.

TABLE 3

CATX-8 cDNA species present in the cDNA libraries
as determined by PCR amplification.

| | | | Library 1: | Library 2: |
|---|---|---|---|---|
| | | | Normal | Cultured |
| | | | Colonic | HCT116 |
| CLONE | 5′ Primer | 3′ Primer | Epithelium | cells |
| CATX-8 | atggggaatggaactgag | cagaccctgaggtgctgt | Full length | Full length and a |
| | [SEQ ID NO: 25] | [SEQ ID NO: 26] | | lower MW species |

CATX-9 [SEQ ID NO: 50] has 162 bp and it is a partial cDNA.

CATX-10 [SEQ ID NO: 51] has 302 bp and it is a partial cDNA.

Tissue Profiling of CATX-2 in Human Tissues

Expression of CATX-2 in human tissues was examined by reverse transcription (3 μg total RNA; 200 U reverse transcriptase (Gibco BRL®, Life Technologies Inc., Rookville, Md.); 50 ng random primer; 50 mM Tris-HCl; pH 8.3; 75 mM KCl; 10 mM DTT; 0.5 mM dNTP in 20 ul) and PCR (94° C. for 1 min; 52° C. for 1 min; 72° C. for 2 min; for 30 cycles). Primer sequences used are given above. CATX-2 is expressed in all normal human tissues examined including liver (1/1), uterus (1/1), breast (1/1), muscle (1/1), and colon mucosa (10/10). CATX-2 was expressed in only 2 out of 15 colonic carcinoma samples, in 0/1 bronchogenic carcinoma brain metastasis, and in 0/1 of hepatocellular carcinoma.

Effect of CATX-2 on Tubulin Polymerization

Functional studies revealed that CATX-2 protein could phosphorylate histone 5 μg, poly-threonine 5 μg, and COOH-APC fragment (1-5 μg), but not poly-Tyr/Glu (5 μg) in vitro (phosphorylation conditions: 50 mM Tris-HCL; pH 7.5; 20 mM MgCl₂ 50 μCi γ-³²p dTP; 1 mM DTT; 1 μg CATX-2; volume of 100 μl; at 30° C. for 30 min). In addition, phosphorylation of carboxyl terminal APC fragment (1-5 μg phosphorylated using cold ATP [1nM]) by CATX-2 protein (1 μg) resulted in an increase in APC's ability to induce tubulin polymerization in vitro (tubulin 200 μg (Cytoskeleton); 80 mM PIPES buffer; pH 6.8; 0.5 mM MgCl₂ 1.0 mM EGTA; 1.0 mM GTP; in 200 μl; absorbance monitored for 1 hour). Thus, CATX-2 appears to have endogenous protein kinase activity for phosphorylation of APC's carboxyl terminal region and this modulates APC's tubulin-polymerizing activity. FIG. 1 shows the effect of CATX-2 on APC's tubulin polymerization activity.

Effect of CATX-8 Overexpression on Culture Colon Cancer Cells

The effect of expressing recombinant CATX-8 in HCT116 cultured colon cancer cells was studied to evaluate the effect of this protein on the progression of these cells through the cell cycle. Over expression of recombinant CATX-8 was accomplished by transfecting HCT116 cells with CATX-8/PcDNA3.1 mammalian expression vector constructs. Controls involved transfection of empty PcDNA3.1 vector. Recombinant HCT116 cells were serum starved in McCoy's media without serum for 72 hours, and then growth stimulated for 20 hours using McCoy's medium with 10% FBS. Flowcytometric analysis was then used to determine the cell cycle distribution of recombinant HCT116 cells. The results (Table 4) show over expression of CATX-8 in HCT116 cells causes more cells to be in GO/G1, G2, and fewer cells to be in S phase.

TABLE 4
Overexpression of CATX-8

| | G₀/G₁ | G₂ | S |
|---|---|---|---|
| HCT116 with empty vector | 48% | 27% | 25% |
| HCT116 with CATX-8 | 56% | 35% | 9% |

These data suggest that CATX-8 may play a role in growth modulation through an effect on the microtubule cytoskeleton. Purified recombinant CATX-8 protein (Ni-column purification via His-tagged CATX-8) can directly induce tubulin polymerization in vitro.

FIG. 2 shows the effect of CATX-8 on tubulin polymerization. Polymerization conditions involved CATX-8 (1-5 μg); tubulin 200 ug (Cytoskeleton); 80 mM. PIPES buffer, pH 6.8; 0.5 mM MgCl₂ 1.0 MM EGTA; 1.0 mM GTP; in 200 ul; absorbance was monitored for 1 hour.

Homology Searches of the Sequences According to the Invention

BLAST2 was used to search the GenBank EST and other databases for sequences having significant homology to the CATX sequences obtained in the present invention. The results are described below.

CATX-1: Thee was no significant homology of CATX-1 to any known sequences in the GenBank database (lowest score=P value of 0.0037). These also was no significant homology to any ESTs (lowest score=P value of 0.06). Homology searches on the translated peptide from CATX-1 using the SWALL database did not show any significant homology, to any known proteins. This indicates that the CATX-1 sequence is novel.

CATX-2: Using BLAST2 to search the GenBank database, CATX-2 was determined to have the greatest homology to a human DNA sequence (AL022151) from clone 199L16 on the X chromosome (HTGS phase I) with a P value of E-105. However, only 54% of CATX-2 (343-975 bp) showed homology with this GenBank sequence. Using-BLAST2 to search the EST database, homology was determined to the ncl7gO4.r1NCI CGAP Pri Homo sapiens cDNA clone (AA226330) with a P value of 7.OOE-28. However, just 17% of the fragment (341-544 bp) showed homology to this EST sequence. Therefore, CATX-2 represents a novel sequence because it contains sequences that have not been reported in the GenBank or EST databases. Moreover, this novel CATX-2 region (1-340) contains the coding region for the predicted protein product for CATX-2.

In addition, based on the homology of CATX-2 with EST (AA806809) in this database, one can predict that the cDNA sequence corresponding to CATX-2 contains the following 3′ segment having a poly-A tail [SEQ ID NO: 52].

Homology search on the translated peptide from CATX-2 using the Non-redundant GenBank CDS translations, SwissProt, and PIR databases did not show any significant homology to any known proteins (lowest score=E value of 0.57). This indicates that the CATX-2 protein is novel. However, one region in CATX-2 (10-26 amino acids shows homology with a domain in several kinases, which indicates that CATX-2 protein is a kinase.

TABLE 5

Comparison of CATX-2 with various
known kinase sequences.

| | | | |
|---|---|---|---|
| | NH₂₋ | | COOH- |
| Kinase | AA | Sequence | AA |

| | | | |
|---|---|---|---|
| CATX-2 | 10 | IDRLEHLHLTEFGL | 23 |
| | | [SEQ ID NO: 53] | |
| Serine/threonine protein | 224 | IDRDGHIKLSDFGL | 237 |
| kinase ORB₆ | | [SEQ ID NO: 54] | |
| Yeast cycle protein | 308 | LDAKGHIKLTDFGL | 321 |
| kinase DBF₂ | | [SEQ ID NO: 55] | |
| Yeast cycle protein | 300 | IDATGHIKLTEFGL | 313 |
| kinase DBF₂₀ | | [SEQ ID NO: 56] | |
| Neuck serine/threonine | 367 | LDRGGHVKLTDFGL | 380 |
| protein kinase COT-1 | | [SEQ ID NO: 57] | |
| Slime mold protein | 4 | mQYGHIKLTEFG | 16 |
| kinase 4 | | [SEQ ID NO: 58] | |
| Slime mold protein | 4 | LDEEGHIKLTDFG | 16 |
| kinase 3 | | [SEQ ID NO: 59] | |

CATX-3: Using BLAST2 to search the GenBank database it was determined that CATX-3 has the highest homology to Homo sapiens DNA sequence (AL021939) from PAC 352A20 on chromosome 6q24.i-25.1 with a P value of 2.00E-52. However, only 61% of CATX-3 (75-208 and 235-288) sequence showed homology with this GenBank sequence. Using BLAST2 to search the EST database, homology was found to the EST sequence yg73d02, s I Soares infant brain 1NIB Homo sapiens cDNA clone 39227 (R51582) with a P value of 1.00E-48. However, only 39% of CATX-3 (83-208) sequence showed homology with this EST sequence. CATX-3 thus contains sequences (1-74 and 209-234), including the region encoding the predicted CATX-3 protein, that have not been reported in the GeenBank or EST databases.

In addition, based on the homology of CATX-3 with- EST (AA602971) in this database, one can predict that the cDNA sequence corresponding to CATX-3 contains a 3′ segment containing a poly-A tail [SEQ ID NO: 60].

Homology searches on the translated peptide from CATX-3 using the SWALL database did not show any significant homology to any known proteins, which indicates that the CATX-3 sequence is novel. However, one segment in CATX-3 (4-23 bp) shows regional homology to a domain contained in protein phosphatase 2C-like protein (2842482) with 13/20 (65%) positives.

CATX-4: Using BLAST2 to search the GenBank database, it was determined that CATX-4 has the highest homology to a Homo sapiens DNA sequence (AC004230) from chromosome 11q12 with a P value of 2.00-E-65. However, only 80% of the CATX-4 (18-144 bp) showed homology with this GenBank sequence. Using BLAST2 to search the EST database, did not develop any significant homology to any known EST sequences. CATX-4 thus contains sequences (1-17 and 145-158), including part of the region encoding the predicted CATX4 protein, that have not been reported in the GenBank or EST databases. CATX-4 has partial homology with CATX-6 (47% identity in 164 residues overlap) and partial homology with CATX-7 (45.9% identity in 157 residues overlap), which indicates that these three sequences may be members of the same family. Homology searches on the translated peptide from CATX-4 using the SWALL database did not show any significant homology to any known proteins.

CATX-5: Using BLAST2 to search the GenBank database, it was determined that CATX-5 has weak homology (P value=3.00E-14) to Human PAC clone DJ515N1 (AC002073) from chromosome 22q1 1. However, only 15% of CATX-5 showed homology with this GenBank sequence. Using BLAST2 to search the EST database, it was determined that CATX-5 has weak homology (P value=2.00E-10) to the EST sequence zg4Of12.sl Soares pineal gland (AA757892). However only 14% of CATX-5 showed homology with this EST sequence. CATX-5 thus contains sequences that have not been reported in the GenBank or EST databases.

Homology searches on the predicted translated peptide from CATX-5 using the SWALL database did not show any significant homology to any known proteins, which indicates that the CATX-5 protein is novel.

CATX-6: Using BLAST2. to search the GenBank database, it was determined that CATX-6 has the highest homology to Homo sapiens CIT-HSP-2340D6.Tr-genomic survey sequence (AQ056648) with a P value of 4.00E-30. However, only 18% of CATX-6 showed homology with this GenBank sequence. Using BLAST2 to search the EST database showed homology to the EST sequence Homo sapiens partial cDNA sequence clone e-lac10:mRNA sequence (P02778) with a P value of 4.00E-28. However, only 17% of CATX-6 showed homology with this EST sequence. CATX thus contains sequences that have not been reported in the GenBank or EST databases. CATX-6 has partial homology with CATX-4(47% identity in 164 residues overlap) and partial homology with CATX-7 (42.9% identity in 310 residues overlap), which indicates that these three sequences may be members of the same family.

In addition, based on the homology of CATX-6 with EST (R60 196) in this database, one can predict that the cDNA sequence corresponding to CATX contains a 3′ segment [SEQ ID NO: 61]. Homology searches (using all 6 possible reading frames) for similarity to any known proteins did not show any significant homology to any known proteins.

CATX-7: Using BLAST2 to search the GenBank database, it was determined that CATX-7 has partial homology to a genomic sequence (AC002110) from chromosome 9q34 with a P value of 1.00E-4I. However, only 24% of CATX-7 showed homology with this GenBank sequence. Using BLAST2 to search the EST database, showed homology to the EST sequence (AA508809) Homo sapiens nh69c09.s1 NCI CGAP Pr8 with a P value of 1.00E-39. However, only 23% of CATX-7 fragment showed homology with this EST sequence. CATX-7 thus contains sequences that have not been reported in the GenBank or EST 12 databases. CATX-7 has partial homology with CATX-6 (45.9% identity in 157 residues overlap) and partial homology with CATX-6 (42.9% identity in 310 residues overlap), which indicates that these three sequences may be members of the same family. Homology searches (using all 6 possible reading frames) for similarity to any known proteins did not show any significant homology to any known proteins.

CATX-8: Using BLAST2 to search the GenBank database, it was determined that CATX-8 has significant homology (P-value=0) to rabbit Rab 25 small GTP-binding protein mRNA (L03303). However, only 56% of CATX-8 fragment showed homology with this GenBank sequence. Using BLAST2 to search the EST database showed significant homology (P value=0) to the EST sequence Homo sapiens. cDNA clone nm79g03.s1 NCI CGAP Co (AA579639). However, only 55% of CAT-X-8 fragment showed homology with this EST sequence. CATX-8 thus contains sequences that have not been reported in the GeiBank or EST databases.

Homology searches on the translated peptide from CATX-8 using the SWALL database revealed significant homology (P value E-103) to rabbit Rab 25 (A48500) small GTP-binding protein (positives=196/206 amino acids [94%]). However there was only one part of CATX-8 (1-206 AA out of the 25.6 total AA) that showed homology with the Rab 25 amino acid sequence (putative length 213 amino acids). CAT-X-8 thus includes human coding sequences that have not been previously reported.

C-ATX-9: Using BLAST2 to search the GenBank database showed no significant homology of CATX-9 to any known genes (lowest score P value of 2.8). Using-BLAST2 to search the EST database, showed no significant homology to any ESTs (lowest score P value of 1.2). CATX-9 thus shows no homology to genes reported in the GenBank or EST databases. Homology searches (using all 6 possible reading frames) for similarity to any known proteins did not show any significant homology to any known proteins.

CATX-10: Using BLAST2 to search the GenBank database showed no significant homology of CATX-10 to any known genes (lowest score=P value of 1.0E-07). Using BLAST2 to search the EST database, showed no significant homology (lowest score P value of 4.0E-05). CATX-10 thus does not have any homology to genes reported in the GenBank or EST databases. Homology searches (using all 6 possible reading frames) for similarity to any known proteins did not show any significant homology to any known proteins.

CATX-11: Using BLAST2 to search the GenBank database, showed no significant homology of CATX-11 to any known genes (lowest score=P value of 0.078). Using BLAST2 to search the EST database, showed significant homology to the EST sequence zqSOfDl.rl Stratagene neuroepithelium (AA205858) Homo sapiens cDNA clone with a P value of 0. However, only 52% of CATX-1 1 (4-527 bp) fragment showed homology with this EST sequence. CATX-1 I thus contains sequences.(1-3 and 527-978 bp), including part of the region encoding the predicted CATX-1 1 protein, that have not been reported in the GenBank or EST databases.

Homology searches on the translated peptide from CATX 12 using the SWALL and proDom databases did not show any significant homology (lowest score=P value of 1.0E-08) to any known proteins.

CATX-13: Using BLAST2 to search the GenBank database, showed no significant homology of CATX-13 to any known genes (lowest score=P value of 1.5). Using BLAST2 to search the EST database, showed no significant homology (lowest score P value of 0.041). CATX-13 thus does not have any homology to genes reported in the GeriBank or EST databases.

Homology searches on the translated peptide from CATX-13 using the SWALL or ProDom databases did not show any Significant homology (lowest score=P value of 0.95) to any known proteins.

CATX-14: Using BLAST2 to search the GenBank database, it was determined that CATX-14 has the highest homology to Homo sapiens DNA sequence (Z78022) from PAC 37M17 on chromosome X with a P value of 1.0E-84. However, only 41% of CATX-14 (86-265 bp) sequence showed homology with this GenBank sequence. Using BLAST2 to search the EST database, showed homology to the EST sequence ze56bo4.rl Soares retina N2b4HR Homo sapiens cDNA clone 362959 (AA018943) with a P value of 9.00E-78. However, only 41% of CATX-14 (86-265 bp) sequence showed homology with this EST sequence. CATX-14 thus contains sequences.(1-85 and 266-418 bp), including part of the region encoding the predicted CATX-14 protein, that have not been reported in the GenBank or EST databases.

Homology searches on the predicted translated peptide from CATX 14 using the SWALL and ProDom databases showed weak homology (3.6E-06) to the mouse TLM oncogene protein (PI7408) with one region in CATX 14 (34-54 amino acids) showing regional homology to a TLM domain (positives=18/21. [84%]), which indicates that CATX-14 may be an oncogene.

CATX-15: Using BLAST2 to search the GenBank database, showed no significant homology of CATX-15 to any known genes (lowest score=P value of 1.1). Using BLAST2 to search the EST database, showed nonsignificant homology (lowest score P value of 0.46). CATX-15 thus has no homology to genes reported in the GenBk or EST databases.

Homology searches on the predicted translated peptide from CAT-X-15 using the SWALL and NRP databases showed some weak homology with DMD human dystrophin (5.0E-05) and with chick chain spectrin protein actin-binding (3.0E-06).

What is claimed is:

1. An isolated nucleic acid comprising a nucleotide sequence of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61 , or a sequence complementary thereto.

2. An isolated nucleic acid comprising a nucleotide sequence which hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto.

3. An isolated nucleic acid comprising a nucleotide sequence having at least about 80% identity to a sequence corresponding to at least about 15 consecutive nucleotides of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ D NO: 44; SEQ ID NO: 46; SEQ ID NO: 48; SEQ ID NO: 50; SEQ ID NO:51; SEQ ID NO: 52; SEQ ID NO: 60; SEQ ID NO: 61, or a sequence complementary thereto.

4. An isolated nucleic acid comprising a nucleotide sequence having at least about 80% identity to a sequence corresponding to one of SEQ ID NO: 27; SEQ ID NO: 29; SEQ ID NO: 31; SEQ ID NO: 33; SEQ ID NO: 35; SEQ ID NO: 37; SEQ ID NO: 38 wherein said nucleic acid sequence encodes a polypeptide or protein which interacts in yeast two-hybrid system with APC.

5. An isolated nucleic acid comprising a nucleotide sequence having at least about 80% identity to a sequence corresponding to one of SEQ ID NO: 40; SEQ ID NO: 42; SEQ ID NO: 44; SEQ ID NO: 46 wherein said nucleic acid sequence encodes a polypeptide or protein which interacts in yeast two-hybrid system with E12.

6. An isolated nucleic acid encoding a polypeptide of one of SEQ ID NO: 28; SEQ ID NO: 30; SEQ ID NO: 32; SEQ ID NO: 34; SEQ ID NO: 36; SEQ ID NO: 41; SEQ ID NO: 43; SEQ ID NO: 45; SEQ ID NO: 47; or SEQ ID NO: 49.

7. The isolated nucleic acid according to claim 1, further comprising a transcriptional regulatory sequence operatively linked to said nucleotide sequence so as to render said nucleotide sequence suitable for use as ;an expression vector.

8. An expression-vector comprising the nucleic acid of claim 4.

9. A host cell transfected with the expression vector of claim 5.

10. A transgenic animal having a transgene comprising a nucleic acid of claim 1 incorporated in cells thereof, which transgene modifies the level of expression of the nucleic acid, the stability of an mRNA transcript of the nucleic acid, or the activity of the polypeptide encoded by the nucleic acid.

11. An isolated nucleic acid which hybridizes under stringent conditions to at least 12 consecutive nucleotides of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ;ID NO: 52; SEQ ID NO: 60; SEQ ID NO: 61.

12. An isolated polypeptide having an amino acid sequence comprising at least 25 amino acids encoded by a nucleic acid of claim 1.

13. A polypeptide of one of SEQ ID NO: 28; SEQ ID NO: 30; SEQ ID NO: 32; SEQ ID NO: 34; SEQ ID NO: 36; SEQ ID NO: 41; SEQ ID NO: 43; SEQ ID NO: 45; SEQ ID NO: 47; or SEQ ID NO: 49.

14. A probe/primer comprising a substantially purified oligonucleotide, said oligonucleotide containing a region of nucleotide sequence which hybridizes under stringent conditions to at least about 12 consecutive nucleotides of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto.

15. An array comprising at least 10 different oligonucleotides of claim 14 attached to a solid support.

16. The probe/primer of claim 15, further comprising a detectable label group attached thereto.

17. The probe/primer of claim 16, wherein said detectable label group is selected from the group consisting of radioisotopes, fluorescent compounds, enzymes and enzyme co-factors.

18. An antibody immunoreactive with a polypeptide of claim 12.

19. An antibody immunoreactive with a polypeptide of claim 13.

20. An antisense oligonucleotide which hybridizes under stringent conditions to at least 12 consecutive nucleotides of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61; or a sequence complementary thereto.

21. A test kit for determining the phenotype of a cell, comprising a probe/primer wherein said test kit is capable of measuring a level of a nucleic acid comprising nucleic acid of one ore more of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto in a sample of cells isolated from a patient.

22. A test kit for determining the phenotype of a cell, comprising a probe/primer wherein said test kit is capable of measuring a level of a nucleic acid hybridizing under stringent conditions to one ore more nucleic acid of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto in a sample of cells isolated from a patient.

23. A test kit for determining the phenotype of a transformed cell, said test kit comprising an antibody specific for a polypeptide comprising amino acid sequence encoded by a nucleic acid which hybridizes under stringent conditions to any one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61.

24. A method of determining the phenotype of a cell, comprising detecting differential expression, relative to a normal cell, of at least one nucleic acid comprising of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46, SEQ ID NO: 48; SEQ ID NO: 50; SEQ ID NO: 51; SEQ ID NO: 52; SEQ ID NO: 60; SEQ ID NO: 61, wherein the nucleic acid is differentially expressed by at least a factor of two.

25. A method of determining the phenotype of a cell, comprising detecting differential expression, relative to a normal cell, of at least one nucleic acid which hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, wherein the nucleic acid is differentially expressed by at least a factor of two.

26. A method for determining the phenotype of cells in a tissue sample from an individual, comprising:
(i.) providing a nucleic acid probe comprising a nucleotide sequence having at least 12 consecutive nucleotides of any of SEQ ID NO: 27; SEQ ID NO: 29; SEQ ID NO: 31; SEQ ID NO: 33; SEQ ID NO: 35; SEQ ID NO:37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto;
(ii.) obtaining a first sample of tissue from an individual;
(iii.) providing a second sample;of tissue which is substantially non-cancerous;
(iv.) contacting the nucleic acid probe with said first and second tissue sample under conditions which allow hybridization of said probe to, said tissue sample; and
(v.) comparing (a) the amount of hybridization of the first tissue sample, with (b) the amount of hybridization of the probe of the second tissue sample, wherein a difference of at least by a factor of two in the amount of hybridization of the probe into the first tissue sample as compared to the amount of hybridization of the second tissue sample is indicative of the phenotype of cells in the first tissue sample.

27. A method of determining the phenotype of a cell, comprising detecting differential expression, relative to a normal cell, of at least one polypeptide comprising amino acid sequence encoded by a nucleic acid which hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ BD NO: 40; SEQ ID NO: 42; SEQ D NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, wherein the polypeptide is-differentially expressed by at least a factor of two.

28. The method of claim 27, wherein the level of said protein is detected in an immunoassay.

29. A method for determining the presence or absence of a nucleic acid which hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto in a cell, comprising contacting the cell with a probe/primer of claim 14.

30. A method for determining the presence of absence of a polypeptide comprising amino acid sequence encoded by a nucleic acid of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42, SEQ ID NO: 44; SEQ ID NO: 46; SEQ ID NO: 48; SEQ ID NO: 50, SEQ ID NO: 51; SEQ ID NO: 52; SEQ ID NO: 60; SEQ ID NO: 61, or a sequence complementary thereto in a cell; comprising contacting the cell with an antibody of claim 18 or 19.

31. A method for determining the presence of absence of a polypeptide encoded by a nucleic acid which hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto in a cell, comprising contacting the cell with an antibody of claim 18 or 19.

32. A method for detecting a mutation in a test nucleic acid which hybridizes under stringent conditions to a nucleic acid of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto or a sequence complementary thereto, comprising
i. collecting a tissue sample from an individual,
ii. isolating nucleic acid from the cells of the tissue sample,
iii. contacting the nucleic acid sample with one or more primers which specifically hybridize to a nucleic acid sequence of at least one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO:44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto under conditions which allow hybridization and amplification of the nucleic acid; and
iv. comparing the presence, absence, or size of an amplification product to the amplification product of a normal cell.

33. A method for identifying an agent which alters the level of expression in a cell of a nucleic acid which-hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, comprising
(i.) providing a cell;
(ii.) treating the cell with a test agent;
(iii.) determining in the cell the level of expression of a nucleic acid which hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto; and
(iv.) comparing the level of expression of the nucleic acid in the treated cell with the level of expression of the nucleic acid in an untreated cell, wherein a change in the level of expression of the nucleic acid in the treated cell relative to the level of expression of the nucleic acid in the untreated cell is indicative of an agent which alters the level of expression of the nucleic acid in a cell.

34. The method of claim 28 where in the nucleic acid is a nucleic acid which hybridizes under stringent conditions to one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto

35. A pharmaceutical composition comprising an agent identified by the method of claim 33 or 34.

36. A pharmaceutical composition comprising a nucleotide sequence of at least one of SEQ ID NO: 27; SEQ ID NO: 29; SEQ ID NO: 31; SEQ ID NO: 33; SEQ-)ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto.

37. A pharmaceutical composition comprising a polypeptide comprising amino acid sequence encoded by a nucleic acid of at least one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60;SEQ ID NO: 61.

38. The method of claim 37 wherein the polypeptide comprises amino acid sequence encoded by a nucleic acid which hybridizes under stringent conditions with at least one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61.

39. A pharmaceutical composition wherein the polypeptide comprises amino acid sequence encoded by a nucleic acid which hybridizes under stringent conditions with at least one of SEQ ID NO: 27; SEQ ID NO: 29; SEQ ID NO: 31; SEQ ID NO: 33; SEQ ID NO: 35; SEQ ID NO: 37; SEQ ID NO: 38; and said polypeptide interacts with APC.

40. A pharmaceutical composition wherein the polypeptide is comprises amino acid sequence encoded by a nucleic acid which hybridizes under stringent conditions with at least one of SEQ ID NO: 40; SEQ ID NO: 42; SEQ ID NO: 44; SEQ ID NO: 46 and said polypeptide interacts with E 12.

41. A method for detecting tumors in which one or more of SEQ ID NO: 27; SEQ ID NO: 29; SEQ ID NO: 31; SEQ ID NO: 33; SEQ ID NO: 35; SEQ ID NO: 37; SEQ ]ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto, are used as probes/primers, said method comprising:
(i.) collecting a sample of cells from an individual,
(ii.) isolating nucleic acid from the cells of the sample,
(iii.) contacting the nucleic acid sample. With one or more primers which specifically hybridize to a nucleic acid sequence of one or more of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto under conditions which allow hybridization and amplification of the nucleic acid; and
(iv.) comparing the presence, absence, or size of an amplification product to the amplification product of a normal cell.

42. The method of claim 41 in which said tumor is colon cancer.

43. A method for detecting tumor in a sample from an individual comprising contacting the sample with an antibody to a protein encoded by at least one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61.

44. The method of claim 43 in which said tumor is colon cancer.

45. A probe for detecting the presence of colon cancer or a precursor of colon cancer, consisting essentially of an isolated nucleic acid of one of SEQ ID NO: 27; SEQ ID NO: 29; SEQ ID NO: 31; SEQ ID NO: 33; SEQ ID NO: 35; SEQ ID NO: 37; SEQ ID NO: 38; SEQ ID NO: 40; SEQ ID NO: 42; SEQ ID NO: 44; SEQ ID NO: 46; SEQ ID NO: 48; SEQ ID NO: 50; SEQ ID NO: 51; SEQ ID NO: 52; SEQ ID NO: 60; SEQ ID NO: 61, or a sequence complementary thereto.


SEQUENCE LISTING (61 sequences)

SEQ ID NO: 1 - DNA, 18 bp, Homo sapiens
agcacttaatattgtaat

SEQ ID NO: 2 - DNA, 18 bp, Homo sapiens
tcaaagccctccagagag

SEQ ID NO: 3 - DNA, 18 bp, Homo sapiens
gggatatagacaggcttg

SEQ ID NO: 4 - DNA, 18 bp, Homo sapiens
tactgcactccaccctgg

SEQ ID NO: 5 - DNA, 18 bp, Homo sapiens
gagattttgatctatgtt

SEQ ID NO: 6 - DNA, 18 bp, Homo sapiens
tcctgaaatcaggtgatc

SEQ ID NO: 7 - DNA, 18 bp, Homo sapiens
gggaggagccagccctgg

SEQ ID NO: 8 - DNA, 18 bp, Homo sapiens
ggtagggttgccaaggtg

SEQ ID NO: 9 - DNA, 18 bp, Homo sapiens
gtgcccaggagtttgaga

SEQ ID NO: 10 - DNA, 18 bp, Homo sapiens
ccaatacctacaaattcc

SEQ ID NO: 11 - DNA, 18 bp, Homo sapiens
gggaggagccagccctgg

SEQ ID NO: 12 - DNA, 18 bp, Homo sapiens
ggtagggttgccaaggtg

SEQ ID NO: 13 - DNA, 18 bp, Homo sapiens
gggaggagccagccctgg

SEQ ID NO: 14 - DNA, 18 bp, Homo sapiens
ggtagggttgccaaggtg

SEQ ID NO: 15 - DNA, 28 bp, Homo sapiens
tgctgggattaggactctttgcctggag

SEQ ID NO: 16 - DNA, 28 bp, Homo sapiens
cgtggcagaggaaaagcccaagttaaag

SEQ ID NO: 17 - DNA, 26 bp, Homo sapiens
cttacatgtgcttggacgggaatgcc

SEQ ID NO: 18 - DNA, 28 bp, Homo sapiens
ctgctcgattgagttaaaagcgctgctg

SEQ ID NO: 19 - DNA, 26 bp, Homo sapiens
caaccccatcaaaaagtgggcaaagg

SEQ ID NO: 20 - DNA, 23 bp, Homo sapiens
ggccggtgatgagcattttttcg

SEQ ID NO: 21 - DNA, 31 bp, Homo sapiens
acactggtggaagaggagagggaagccaaac

SEQ ID NO: 22 - DNA, 26 bp, Homo sapiens
ccacatgccgcaatgagttcagaacc

SEQ ID NO: 23 - DNA, 12 bp, Artificial sequence
  Oligonucleotide primer
  misc_feature: Antisense primer for amplification of colonic epithelial library
ccgccatcgctc

SEQ ID NO: 24 - DNA, 17 bp, Artificial Sequence
  Oligonucleotide primer
  misc_feature: Sense primer for amplification of colonic epithelial library
taccactacaatggatg

SEQ ID NO: 25 - DNA, 18 bp, Homo sapiens
atggggaatggaactgag

SEQ ID NO: 26 - DNA, 18 bp, Homo sapiens
cagaccctgaggtgctgt

SEQ ID NO: 27 - DNA, 471 bp, Homo sapiens
gcacttaatattgtaattggttttccagcgctttctctaagtttctgctcgattgagttaaaagcgctgctgtccttaatttgcttaaggcttcaaacacgatctaaaagaagtggtgcacagagctctcgtcatgtgagaacaattcaatgtgatgtcttgccaggaatgataaactctggtgacttgataaatctcagagttcagatttcagatgaagttagctttcagcctggcattcccgtccaagcacatgtaaggctccgcaaaattgatgacaccataaaggagtccagtttccaccaccacgatgctccgtcctcaaagaggagcacatcccagtcgcctctctctggagggctttgaatgcctctctgatcagtcaacaggacccttgaatgagcacttattcattgtgccagtaatcttttcagaggacagcagtggcagcaggtagtacatacttactag

SEQ ID NO: 28 - protein, 121 aa, Homo sapiens
ALNIVIGFPALSLSFCSIELKALLSLICLRLQTRSKRSGAQSSRHVRTIQCDVLPGMINSGDLINLRVQISDEVSFQPGIPVQAHVRLRKIDDTIKESSFHHHDAPSSKRSTSQSPLSGGL

SEQ ID NO: 29 - DNA, 983 bp, Homo sapiens
gcggacctgcagcctgtttcaaaataatgatctataccagttgacttagagtgatttccacgatgctctggtgaaaaagaaaagttagttttagagaagacgctcatgaattcgcggccgcgtcgacgggatatagacaggcttgagcacctccacctcacagaatttggactaaaattggggcaaaaaatatttttgttaaacaaaagagattttattttatttttatcattattttgaaacaggttcttgctctgtcacccagggtggagtgcagtagcacaaggtgcatcccgggatcaagccttcctcctgtctcagcctccccagtagctgagactgatacaggaggtagaaagaaattatttaggcagatagtgagggcaaatgagtcctcagcagagcttcctttctaacaaaaagcagcccaagaaattagttttctcctaacaaaaagtagcctgaaaaatctagctgcaaacataggtaagcaagctggaacattgcacaggggaatgctggcagctgtgtcaatagaaaagggtactgggggccaggcatgtccatcatggaagcttcatcttcctttttgttagcacgtgtacagtaagaaagaagtgggcaacatggcacagcttaggctgaaaacccgcctgcataataaaatactggggtgggggctgccagaaaatcgtgccccatgcaaatggcacacctggtcctaaccagctttttgtgccctacgtagatcagacacaactttccaaccagctcatctataaaaccttctgcatttcaccgtgggtcgacaacccatttttctgggacccctctctgtagcagagagctattttctttcttttgcctattaaacttcctgctgttaacctcactcttgtggtgtccatgtccttgatctctgtggctgtgagacaatgaacctcaggttttaccccagacaatgaggctgcttcaagactaca

SEQ ID NO: 30 - protein, 44 aa, Homo sapiens [sequence omitted]

SEQ ID NO: 31 - DNA, 301 bp, Homo sapiens
  misc_feature (44)..(44): unknown
  misc_feature (195)..(195): unknown
gagattttgatctatgttatacagtaaccacaacaaataggtanagcacagaattgattaaaaatatgaagtcagccagttgtggtggctcacacctgtaataccagcactttgggaggctgaggcaggcagatcacctgaggtcaggagttcaaaagcagcctggccaacatggtgaaccccatctctactaanaatacaaaaattttcccatgcgcagtgatatgtgcatataatcccagctactcaggaggctgacacagaagaatcactttgaacccgggaggcggagttgtaatga

SEQ ID NO: 32 - protein, 32 aa, Homo sapiens
  MISC_FEATURE (14)..(14): X is any amino acid
DFDLCYTVTTTNRXSTELIKNMKSASCGGSHL

SEQ ID NO: 33 - DNA, 158 bp, Homo sapiens
cgggggggaggagccagccctggccccagggctggctccacttccacgctccttaccaagctacccttaccaagctacgggtgcaggaagcctttagatgcctcgctctccctgcccctccacacttggtatgctgtgtccaccttggcaaccctacc

SEQ ID NO: 34 - protein, 52 aa, Homo sapiens
RGGGASPGPRAGSTSTLLTKLPLPSYGCRKPLDASLSLPLHTWYAVSTLATL

SEQ ID NO: 35 - DNA, 290 bp, Homo sapiens
gtgcccaggagtttgagaccagcttgggcaacataataagaccctcatgtcttaaaaaaataaaaaaaataaaaactaaaaactttgtgtattatatatgctcagtaaatgatagttctcttatctttcccactattattggttttaaagaatggtgtagacaggttttccttaaatggcctctgttgttctcctttctcccaaaatattcattattaattttgtctaattttcattaagaaatttgtaggtattggagtaaacctttgttcacttcttttttttttttt

SEQ ID NO: 36 - protein, 78 aa, Homo sapiens
AQEFETSLGNIIRPSCLKKIKKIKTKNFVYYICSVNDSSLIFPTIIGFKEWCRQVFLKWPLLFSFLPKYSLLILSNFH

SEQ ID NO: 37 - DNA, 520 bp, Homo sapiens
  misc_feature (96)..(96): Unknown
  misc_feature (241)..(241): Unknown
  misc_feature (248)..(248): Unknown
  misc_feature (214)..(214): Unknown
ccacataggtggtacacaggtgcccaaagggcctctcccaccaggactgcacttcaaactcccccatgcactgacattaacagtggaaaggccagnctgcttggggtgaattgtcatacaattagcatttgtaaaagttaagctaaattacaaaaattaaaaaaaatttaaaggacatgtaagtggaatttaatataccctggacctgggtgtncctcaaaatgccagcctcagccttctncctgganaactcactaaaaggccacgggaaagggagcagggcagccccctgggaagcaggatggcgtggcatccagggctgggtgggggtgctgagctttggtaaaacaatgcaaggtcctccttcctggtgttctcaccacattcaccctgaagtgcatgttatgaccttggctccctgcagcctcaacctcctgggctcaagcaatcctcctacctcagcctccccgagtaactgggactacaggtgcacaccaccacacctggctaatttgtaaat

SEQ ID NO: 38 - DNA, 410 bp, Homo sapiens
  misc_feature (121)..(121): Unknown
  misc_feature (134)..(134): Unknown
  misc_feature (148)..(148): Unknown
  misc_feature (150)..(150): Unknown
  misc_feature (164)..(164): Unknown
actgcagccccagctgacatctgactgcaacctcgtgaggagcaataatgcacacgaaatggtgacaacagagtggggtgggtggctcacgcctgtaatcccagcacttttgagaggccanggcaggtggatcncctgaggtcaggantnccaaaacagcctgnccaacatggtgaaaccctgtctcttactaaaaaaaaatacaaaaattagccaggcatggtggtgcaagtcccagctactcaggaggctgaggcaggagaattgcttgaacccgggaggcagaggttgcactgagccaaaattgcaccactgcactccagcctgggcgacaaaggggactcaaaaaaaaaaaagaggctggactcttctcttgcacctctattcatcgtgggccaggggtggtgtca

SEQ ID NO: 39 - DNA, 1019 bp, Homo sapiens
  misc_feature (951)..(951): Unknown
ccatcctaatacgactcactatagggctcgagcggccgcccgggcaggtttgaaactgagtggcccacgatgggaagaggggaaagcccaggggtacaggaggcctctgggtgaaggcagaggctaactggggttcggagcgaccttggccgttggctgaccatctttgtgctgtctgtcgtcactatcatcatctgcttcacctgctcctgctgctgcctttacaagacgtgccgccgaccacgtccggttgtcaccaccaccacatccaccactgtgtgcatgccccttatcctcagcctccaagtgtgccgcccagctaccctggaccaagctaccagggctaccacaccatgccgcctcagccagggatgccagcagctaggactgggagagataggagaagtaggactgctgtgattaggacggatcagacgagaggggcgtttggtattgggttatggcagggggttttatattgataattgttgtgatgaaattgatggcccctaagatagagacacctgctaggtgtaaggagaagatggttaggtctacggaggctccagggtgggagtagttccctgctaaggagggtagactgttcaacctgttcctgctccggcctccactatagcagatgcgagcaggagtaggagagagggaggtaagagtcagaagcttatgttgtttatgcggggaaacgccatatcggggcacgattattaggggaactagtcagttgccaaagcctccgattatgatgggtattactaatgaagaagattattacaaatgcatgggctgtgacgataacgttgtagatgtggtcgttacctagaaggttgcctggctggcccagctcggctcgaataaggaggcttagagctgtgcctaggactccagctcatgcgccgaataataggtatagtgttccaatgtctttgtggnttgtagagaatagtcaacgacctccccgggcggccgctcgagccctatagtgagtcgtattaggatgg

SEQ ID NO: 40 - DNA, 978 bp, Homo sapiens
  misc_feature (3)..(3): unknown
  misc_feature (20)..(20): unknown
  misc_feature (26)..(26): unknown
  misc_feature (40)..(40): unknown
  misc_feature (82)..(82): unknown
tanagaactgagggtcagantgaccntgcctcatagtatncgatcagattcagaagatatctgtttatttacgaaggatgancccaattcaactcctgaaaagacagaacagttttatagaaagcttttaaacaagcatggaattaaaaccgtttctcagattatctccctccaaactctaaagaaggaatataaatcctatgaagccaagctccgccttctgagcagttttgatttcttccttactgatgccagaattaggcggctcttaccctcactcattgggagacatttctatcaaagaaagaaagttccagtatctgtaaaccttctgtccaagaatttatcaagagagatcaatgactgtataggtggaacagtcttaaacatttctaaaagtggttcttgcagtgctatacgtattggtcacgttggaatgcaaattgagcacatcattgaaaacattgttgctgtcaccaaaggactttcagaaaaattgccagagaagtgggagagcgtgaaactcctgtttgtgaaaactgagaaatcggctgcacttcccatcttttcctcgtttgtcagcaattgggatgaagccaccaaaagatctttgcttaataagaagaaaaaagaggcaaggagaaaacgaagagaaagaaattttgaaaaacaaaaggagaggaagaagaagaggcagcaggctaggaagactgcatcagttcttagtaaagatgatgtggcacctgaaagtggtgatactacagtgaagaaacctgaatcaaagaaggaacagaccccagagcatgggaagaaaaaacgtggcagaggaaaagcccaagttaaagcaacaaatgaatccgaagacgaaatcccacagctggtaccaataggaaagaagactccagctaatgaaaaagtagagattcaaaaacatgccacaggaaagaagtctccaggcaaagagtcctaatcccagca

SEQ ID NO: 41 - protein, 322 aa, Homo sapiens
  MISC_FEATURE (1)..(1): X is any amino acid
  MISC_FEATURE (7)..(7): X is any amino acid
  MISC_FEATURE (9)..(9): X is any amino acid
  MISC_FEATURE (13)..(13): X is any amino acid
  MISC_FEATURE (27)..(27): X is any amino acid
XELRVRXTXPHSXRSDSEDICLFTKDXPNSTPEKTEQFYRKLLNKHGIKTVSQIISLQTLKKEYKSYEAKLRLLSSFDFFLTDARIRRLLPSLIGRHFYQRKKVPVSVNLLSKNLSREINDCIGGTVLNISKSGSCSAIRIGHVGMQIEHIIENIVAVTKGLSEKLPEKWESVKLLFVKTEKSAALPIFSSFVSNWDEATKRSLLNKKKKEARRKRRERNFEKQKERKKKRQQARKTASVLSKDDVAPESGDTTVKKPESKKEQTPEHGKKKRGRGKAQVKATNESEDEIPQLVPIGKKTPANEKVEIQKHATGKKSPGKES

SEQ ID NO: 42 - DNA, 326 bp, Homo sapiens
  misc_feature (190)..(190): Unknown
  misc_feature (301)..(301): Unknown
  misc_feature (304)..(304): Unknown
gtttctgctcgattgagttaaaagcgctgctgtccttaatttgcttaaggcttcaaacacgatctaaaagaagtggtgcacagagctctcgtcatgtgagaacaattcaatgtgatgtcttgccaggaatgataaacttggtgacttgataaatctcagagttcagatttcagatgaagttagctttcancctggcattcccgtccaagcacatgtaaggctccgcaaaattgatgacaccataaaggagtccagtttccaccaccacgagtctccgtcctcaaagaggagcacatcccantcncctctctctggagggctttgac

SEQ ID NO: 43 - protein, 48 aa, Homo sapiens
FCSIELKALLSLICLRLQTRSKRSGAQSSRHVRTIQCDVLPGMINLVT

SEQ ID NO: 44 - DNA, 418 bp, Homo sapiens
  misc_feature (272)..(272): Unknown
agggttgcattattttatattctcaccaggaattatgaagctttccatttctccacatcctcatcaacattagttattacttgtcttttaatgatcaccattctaactggtgtgagatggtatctcattgtggttttgatttgcatttctctgatggccggtgatgatgagcattttttcgtgtgtctgttggctgcataaatgtcttcgtttgcgaagtgtctgttcatatcctttgcccactttttgatggggttgtttgtttacattanctttctgacatcatttaaaatattaaacgctttaatatatggcacctttaaaattgagtcagagccttttagtgaaagaatctgttttccctaaaatagaaataactttaatatttaatagcttcaggtttatacactccattttt

SEQ ID NO: 45 - protein, 66 aa, Homo sapiens
RVALFYILTRNYEAFHFSTSSSTLVITCLLMITILTGVRWYLIVVLICISLMAGDDEHFFVCLLAA

SEQ ID NO: 46 - DNA, 2031 bp, Homo sapiens
  misc_feature (1162)..(1162): Unknown
  misc_feature (1202)..(1202): Unknown
  misc_feature (1229)..(1229): Unknown
  misc_feature (1277)..(1277): Unknown
  misc_feature (1885)..(1885): Unknown
  misc_feature (1889)..(1889): Unknown
  misc_feature (2013)..(2013): Unknown
ttgccatgctgacagaaacacaatcaatcatctctcagctcccgccccagcccttgaatatgatactctaaggcagcagcaggaagaacatcggcaactgcgtgagttgatagctgaacacaagcctcatatagataagatgaacaaaactggccacagtactgaatgagccctggggaaggcttttctatccaagagaagtatgtggcagccgacaccctttacagtcacattaaagaagatgtcaaaaagcgtgctgtggcactggatgaagccatttctcaatcaactcagttccatgacaagatagatcagatccttgagagcctggaacgcatcgtggaacgtctgaggcagccaccctctatctctgcagaggttgagaagatcaaggaacagatcagtgaaaataagaatgtgtcagtagacatggaaaagctacagccgttgtatgaaactcttaaacagaggggagaggaaatgattgctagatctggggggactgataaagacatatctgccaaagctgttcaggataagcttgaccaaatggttttcatttgggagaacatacacacactggtggaagagagggaagccaaactactggatgtgatggagctagcagaaaagttctggtgtgatcacatgtcattgatagttaccattaaagatactcaagatttcatccgggacctggaagatcctggaattgatccttcagtagtaaaacaacagcaagaagcagcagagaccataagggaagaaatagatggactacaggaggagctggatatagttattaacctaggttctgaactcattgcggcatgtggggagcctgataaacccattgtcaagaagagtatacatgagttaaattcagcgatgggattctctaaataaagcttggaaagaccggattgaccaacttgagagcaatgcagctgccgtcagtacagatggactgcaggcggtatttttctgggtagatattgcaggtggtaagttcgcttcaatgtctccaattggaacagcatctcgaaactgtcaagcagcagattgatgtgctagagcaatttaagtctgaggcctatcaacagcagatagagtggtacgactgactcatcacgcagacgcttttgctaaagaaagtaacagnagagagtgacaaacacactgttctagacccaaaaatgganctgatattgatatggctagcctggagnagagaatcatcaacagacagcataaactggagggtgctctattagccntgggtcagttccaacatgccctgtatgagctcctgcatgctgacacacaccgagggcttgctaagtgagcagaaacctgttggaggagaccctaaagccattgaaattgaacttgccaagcatcatgtgctccaaaatgatgtattagcccatcagtccacagtggaagccgttaataaagcaggaaatgatctaattgaatcaagtgcaggagaagaagcaagcaaccttcagaacaagctagaggttttaaatcaacgctggcaaaatgttttggaaaaaacagaacaaaggaagcagcagctggatggtgccttgcgccaggccaaagggttccatggcgaaattgaggatttgcagcagtggctgactgacacggagcgtcatctgttggcatctaaaccgctgggaggtttaccggaaacagccaaggagcagcttaatgtccatatggaagtctgtgctgcctttgaagctaaagaagaaacatataagagtctgatgcagaaaggccagcagatgcttgcaagatgcccaaaatctgcagagacaaatattgaccaagacataaataacttgaaaaaaaaaaaaaaaaaaaaaaaaaaaanacangccgctgaattctagaattcagcggccgctgaattctagacctgcccgggcggcgcgctcgagccctatagtgagtcgtattaggatggaatcactagtcggccgcctgcaggtcgaccatatnggagagctcccaacgcgt

SEQ ID NO: 47 - protein, 246 aa, Homo sapiens
MSPGEGFSIQEKYVAADTLYSHIKEDVKKRAVALDEAISQSTQFHDKIDQILESLERIVERLRQPPSISAEVEKIKEQISENKNVSVDMEKLQPLYETLKQRGEEMIARSGGTDKDISAKAVQDKLDQMVFIWENIHTLVEEREAKLLDVMELAEKFWCDHMSLIVTIKDTQDFIRDLEDPGIDPSVVKQQQEAAETIREEIDGLQEELDIVINLGSELIAACGEPDKPIVKKSIHELNSAMGFSK

SEQ ID NO: 48 - DNA, 1085 bp, Homo sapiens
cacccccacctgccagagccttgatccctccctaggccctgcctaaccttgagttggccccaatccctcttggctgcagaagtccctttcacccccaatgagaggaggggcaggaccagatcttttgagagctgagggttgagggcattgagcaacacacagatttgtcgcctctgtccccgaagacacctgcaccctccatgcggagccaagatggggaatggaactgaggaagattataactttgtcttcaaggtggtgctgatcggcgaatcaggtgtggggaagaccaatctactctcccgattcacgcgcaatgagttcagccacgacagccgcaccaccatcggggttgagttctccacccgcactgtgatgttgggcaccgctgctgtcaaggctcagatctgggacacagctggcctggagcggtaccgagccatcacctcggcgtactatcgtggtgcagtgggggccctcctggtgtttgacctaaccaagcaccagacctatgctgtggtggagcgatggctgaaggagctctatgaccatgctgaagccacgatcgtcgtcatgctcgtgggtaacaaaagtgacctcagccaggcccgggaagtgcccactgaggaggcccgaatgttcgctgaaaacaatggactgctcttcctggagacctcagccctggactctaccaatgttgagctagcctttgagactgtcctgaaagaaatctttgcgaaggtgtccaagcagagacagaacagcatccggaccaatgccatcactctgggcagtgccaggctggacaggagcctggcctggggagaagagggcctgttgcatcagcctctgaccttggccagcaccacctgcccccactggctttttgtgccccttgtccccacttcagccccaggacctttccttgccctttggttccagatatcagactgttccctgttcacagcaccctcagggtcttaaggtcttcatgccctatcacaaatacctcttttatctgtccacccctcacagactaggaccctcaaataaagctgttttatatcaaaaaaaaaaaaaaaaaa

SEQ ID NO: 49 - protein, 256 aa, Homo sapiens
MGNGTEEDYNFVFKVVLIGESGVGKTNLLSRFTRNEFSHDSRTTIGVEFSTRTVMLGTAAVKAQIWDTAGLERYRAITSAYYRGAVGALLVFDLTKHQTYAVVERWLKELYDHAEATIVVMLVGNKSDLSQAREVPTEEARMFAENNGLLFLETSALDSTNVELAFETVLKEIFAKVSKQRQNSIRTNAITLGSARLDRSLAWGEEGLLHQPLTLASTTCPHWLFVPLVPTSAPGPFLALWFQISDCSLFTAPSGS

SEQ ID NO: 50 - DNA, 162 bp, Homo sapiens
aagggaaagattagggtcaagagtatgatttaggagactgtgcaccattcatatgcaagtggatgcacaattcctgaggcagtgccacagggagggatggagaggctggactcaagagtcagtgcaggagaatatctaaacttggtgcttgagcgatggcgg

SEQ ID NO: 51 - DNA, 301 bp, Homo sapiens
  misc_feature (12)..(12): unknown
  misc_feature (15)..(15): unknown
  misc_feature (16)..(16): unknown
  misc_feature (18)..(18): unknown
  misc_feature (36)..(36): unknown
  misc_feature (41)..(41): unknown
  misc_feature (51)..(51): unknown
  misc_feature (53)..(53): unknown
  misc_feature (107)..(107): unknown
  misc_feature (158)..(158): unknown
  misc_feature (168)..(168): unknown
  misc_feature (171)..(171): unknown
  misc_feature (197)..(197): unknown
  misc_feature (199)..(199): unknown
  misc_feature (255)..(255): unknown
agcatgggcagngcnncngtaatctcgctaacattngtgtntttttctgtntnggtttctctttgtgcaagtgtgtatgtacatctactaggtgtgaaatggtatcncgctgtggtttccactagcattttcctgatggtagtgatggtgatcatctncttatgtacntantgacgaattgtatatcttcttcagananaatgtctgttcagatcctttgaccatttttaaatttggttgtttgtctgttattantgagtgtctggaagtcacgccactatggacctgcattatataggga

SEQ ID NO: 52 - DNA, 1024 bp, Homo sapiens
gcggacctgcagcctgtttcaaaataatgatctataccagttgacttagagtgatttccacgatgctctggtgaaaaagaaaagttagttttagagaagacgctcatgaattcgcggccgcgtcgacgggatatagacaggcttgagcacctccacctcacagaatttggactaaaattggggcaaaaaatatttttgttaaacaaaagagattttattttatttttatcattattttgaaacaggttcttgctctgtcacccagggtggagtgcagtagcacaaggtgcatcccgggatcaagcctcctcctgtctcagcctccccagtagctgagactgatacaggaggtagaaagaaattatttaggcagatagtgagggcaaatgagtcctcagcagagcttcctttctaacaaaaagcagcccaagaaattagttttctcctaacaaaaagtgcctgaaaaatctagctgcaaacataggtaagcaagctggaacattgcacaggggaatgctggcgctgtgtcaatagaaaagggtactgggggccaggcatgtccatcatggaagcttcatcttccttttgttagcacgtgtacagtaagaaagaagtgggcaacatggcacagcttaggctgaaaacccgcctgcataataaaatactggggtgggggctgccagaaaatcgtgccccatgcaaatggcacacctggtcctaaccagctttttgtgccctacgtagatcagacacaactttccaaccagctcatctataaaaccttctgcatttcaccgtgggtcgacaacccatttttctgggacccctctctgtagcagagagctattttctttcttttgcctattaaacttcctgctgttaacctcactcttgtggtgtccatgtccttgatctctgtggctgtgagacaatgaacctcaggttttaccccagacaatgaggctgcttcagtgggagggtgttttgggctcacaagtagataaaaaggagaaatgaaaatgag

SEQ ID NO: 53 - protein, 14 aa, Homo sapiens
IDRLEHLHLTEFGL

SEQ ID NO: 54 - protein, 14 aa, Homo sapiens
IDRDGHIKLSDFGL

SEQ ID NO: 55 - protein, 14 aa, Yeast
LDAKGHIKLTDFGL

SEQ ID NO: 56 - protein, 14 aa, Yeast
IDATGHIKLTEFGL

SEQ ID NO: 57 - protein, 14 aa, Neuck
LDRGGHVKLTDFGL

SEQ ID NO: 58 - protein, 13 aa, Slime mold
IDQYGHIKLTEFG

SEQ ID NO: 59 - protein, 13 aa, Slime mold
LDEEGHIKLTDFG

SEQ ID NO: 60 - DNA, 803 bp, Homo sapiens
  misc_feature (96)..(96): unknown
  misc_feature (214)..(214): unknown
  misc_feature (241)..(241): unknown
  misc_feature (248)..(248): unknown
  misc_feature (555)..(555): unknown
  misc_feature (577)..(577): unknown
  misc_feature (628)..(628): unknown
  misc_feature (647)..(647): unknown
  misc_feature (760)..(760): unknown
ccacataggtggtacacaggtgcccaaagggcctctcccaccaggactgcacttcaaactcccccatgcactgacattaacagtggaaaggccagnctgcttggggtgaattgtcatacaattagcatttgtaaaagttaagctaaattacaaaaattaaaaaaaatttaaaggacatgtaagtggaatttaatataccctggacctgggtgtncctcaaaatgccagcctcagccttctncctgganaactcactaaaaggccacgggaaagggagcagggcagccccctgggaagcaggatggcgtggcatccagggctgggtgggggtgctgagctttggtaaaacaatgcaaggtcctccttcctggtgttctcaccacattcaccctgaagtgcatgttatgaccttggctccctgcagcctcaacctcctgggctcaagcaatcctcctacctcagcctccccgagtaactgggactacaggtgcacaccaccacacctggctaatttgtaaaggaaatcctgccttcctttaggaaatgggtttgaanctggaataagaggaaaaaaaangccctttcaagccccaggcctgggaaaacgccctttctcccaggtgccctntccctcacccagctcctgncaccctctggggagccttccccaccttagctgtctcctgcccccagggagggatggaggagataatttgcttatattaaaaacaaaaaatggctgaggcaggagtttgggacnagcctgggctatatagcaagaccccatcactacaaatttttta

SEQ ID NO: 61 - DNA, 803 bp, Homo sapiens; identical to SEQ ID NO: 60
  misc_feature (96)..(96): unknown
  misc_feature (214)..(214): unknown
  misc_feature (241)..(241): unknown
  misc_feature (248)..(248): unknown
  misc_feature (555)..(555): unknown
  misc_feature (577)..(577): unknown
  misc_feature (628)..(628): unknown
  misc_feature (647)..(647): unknown
  misc_feature (760)..(760): unknown